SEQUENCE LISTING (52 sequences)

SEQ ID NO: 1 - DNA, 1942 bp, Aspergillus niger
  sig_peptide 1..45
  exon 46..1766
  intron 1767..1821
  exon 1822..1942
  source 1..1942: mol_type = genomic DNA
atgaagcttcaaacggcttccgtactgctcggcagtgctgctgctgcctcgccttccatgcagacgcgggcctccgttgtcatcgactacaatgtcgctcctccaaacctatccactctgcccaatggctccctcttcgaaacatggcgtccccgcgcccacgtcctgccccccaacggccagatcggtgacccctgcctgcattacaccgatccctccacgggcctcttccacgtcggcttccttcacgatggcagcggcatctccagcgccaccactgatgatctagccacctacaaggacctcaaccaaggcaaccaagtcattgttcccgggggtatcaacgaccccgtcgccgtcttcgatggctccgtcatccccagcggcatcaacggcctccccactctcctctacacctccgtctcctacctccccatccactggtctatcccctacacccgtggcagtgagacccaatccctcgctgtctcctcggacggcggcagcaacttcaccaagctcgaccagggccccgtcatccctggccctcccttcgcctacaacgtcaccgcattccgggacccctacgtcttccaaaaccccaccctcgactccctcctgcacagcaagaacaacacctggtataccgtcatctccggcggtctgcacggcaagggccccgcccagttcctctaccgccagtacgacccggacttccagtactgggagttcctcggccaatggtggcacgaacccaccaactccacctggggtaacggcacctgggccggccgatgggccttcaacttcgaaaccggcaacgtcttcagtctcgatgagtacggatacaacccccacggccagatcttctccacgatcggcaccgagggctctgaccagcccgtcgtgccccagctcaccagcatccacgacatgctctgggtgtccggcaacgtctctcgcaacggttctgtctcgttcaccccgaacatggcgggcttcctcgactggggcttctcctcttacgcagctgccggaaaggtcctcccctcgacttctctgccctcgacgaagagcggcgccccggaccgcttcatctcgtacgtctggctgtccggtgacctgttcgaacaggccgaaggattccccacgaaccagcagaattggaccggtacgctgttgcttccgcgagagttgcgcgtgctgtatatccccaacgtggtggacaatgctctggctcgggaatctggtgcctcgtggcaggtcgtgagcagcgatagcagtgcgggcaccgtcgagctgcagaccctgggtatctccattgcccgggaaaccaaggccgccctgctgtcgggaacgtcgttcaccgagtccgaccgtactctgaacagcagtggtgttgtgccgttcaagcgctccccgtccgagaagttctttgttttgtccgcgcagctgtccttccctgcttcggctagggggtcgggacttaagagtggattccagatcctctcgtcggagctggaaagcaccactgtctactaccagttctcgaatgagtcgattattgtcgaccgcagtaacaccagtgctgcggcccgcaccacggatggtatcgatagcagtgcggaggctggcaagttgcgcctgtttgacgtgttgaatgacggagagcaggcgattgagacgttggatttgactctcgtggtggataactcggtattggagatctatgccaatggtcgctttgcgttgagtacttgggttcggtgagtatcatcctaatttatgaatgaagtgtttatgatgctaacgtggggatagttcttggtacgccaattccacgaacatcagtttcttccagaatggcgtgggtggtgttgcgttctccaacgtgactgtttccgagggcttgtatgatgcttggccggatcgtcagtcttga

SEQ ID NO: 2 - protein, 628 aa, Aspergillus niger
  SIGNAL 1..15
  PEPTIDE 16..628
MKLQTASVLLGSAAAASPSMQTRASVVIDYNVAPPNLSTLPNGSLFETWRPRAHVLPPNGQIGDPCLHYTDPSTGLFHVGFLHDGSGISSATTDDLATYKDLNQGNQVIVPGGINDPVAVFDGSVIPSGINGLPTLLYTSVSYLPIHWSIPYTRGSETQSLAVSSDGGSNFTKLDQGPVIPGPPFAYNVTAFRDPYVFQNPTLDSLLHSKNNTWYTVISGGLHGKGPAQFLYRQYDPDFQYWEFLGQWWHEPTNSTWGNGTWAGRWAFNFETGNVFSLDEYGYNPHGQIFSTIGTEGSDQPVVPQLTSIHDMLWVSGNVSRNGSVSFTPNMAGFLDWGFSSYAAAGKVLPSTSLPSTKSGAPDRFISYVWLSGDLFEQAEGFPTNQQNWTGTLLLPRELRVLYIPNVVDNALARESGASWQVVSSDSSAGTVELQTLGISIARETKAALLSGTSFTESDRTLNSSGVVPFKRSPSEKFFVLSAQLSFPASARGSGLKSGFQILSSELESTTVYYQFSNESIIVDRSNTSAAARTTDGIDSSAEAGKLRLFDVLNDGEQAIETLDLTLVVDNSVLEIYANGRFALSTWVRSWYANSTNISFFQNGVGGVAFSNVTVSEGLYDAWPDRQS

SEQ ID NO: 3 - protein, 613 aa, Aspergillus niger
ASPSMQTRASVVIDYNVAPPNLSTLPNGSLFETWRPRAHVLPPNGQIGDPCLHYTDPSTGLFHVGFLHDGSGISSATTDDLATYKDLNQGNQVIVPGGINDPVAVFDGSVIPSGINGLPTLLYTSVSYLPIHWSIPYTRGSETQSLAVSSDGGSNFTKLDQGPVIPGPPFAYNVTAFRDPYVFQNPTLDSLLHSKNNTWYTVISGGLHGKGPAQFLYRQYDPDFQYWEFLGQWWHEPTNSTWGNGTWAGRWAFNFETGNVFSLDEYGYNPHGQIFSTIGTEGSDQPVVPQLTSIHDMLWVSGNVSRNGSVSFTPNMAGFLDWGFSSYAAAGKVLPSTSLPSTKSGAPDRFISYVWLSGDLFEQAEGFPTNQQNWTGTLLLPRELRVLYIPNVVDNALARESGASWQVVSSDSSAGTVELQTLGISIARETKAALLSGTSFTESDRTLNSSGVVPFKRSPSEKFFVLSAQLSFPASARGSGLKSGFQILSSELESTTVYYQFSNESIIVDRSNTSAAARTTDGIDSSAEAGKLRLFDVLNDGEQAIETLDLTLVVDNSVLEIYANGRFALSTWVRSWYANSTNISFFQNGVGGVAFSNVTVSEGLYDAWPDRQS

SEQ ID NO: 4 - DNA, 2948 bp, Aspergillus niger
  sig_peptide 1..57
  intron 58..153
  exon 154..296
  intron 297..347
  exon 348..392
  intron 393..446
  exon 447..499
  intron 500..550
  exon 551..1742
  intron 1743..1804
  exon 1805..2663
  intron 2664..2714
  exon 2715..2948
  source 1..2948: mol_type = genomic DNA
atgaggttcactttgatcgaggcagtggctctgactgccgtctcgctggccagcgctgtacgtgccgtcacttccttcctcgtgtgaattgcaattgtgctcgattggattcacttctttgtttcatcaccactaacaaatgtctattcaaaggatgaattggcctactcccctccgtattacccctccccttgggccaatggccagggtgactgggcggaagcataccagcgcgctgttgatatcgtctcgcagatgacattggctgagaaggtcaatttgactacgggaactgggtaaggctcaatggcacaaacgatgtgtatgctcctgctaacgacctccagatgggaattggaattatgtgttggtcagactggaggtgttccccggtaagtttgtgaagattgtcaaaacagggagcatttactgatatatggtgacagattgggaattccgggaatgtgtgcacaggatagccctctgggtgttcgtgactgtaagcaacccggtgtttttaggctttgatgctcttgctgacacagcgtagccgactacaactctgcgttccccgccggtgtcaacgtggccgcaacctgggacaagaatctggcttaccttcgtggccaggctatgggtcaggagtttagtgacaagggtgctgatatccaattgggtccagctgccggccctctcggtagaagtcccgacggcggtcgtaactgggagggcttctcccccgacccggccctcagtggtgtgctctttgcagagacaatcaagggtattcaggatgctggtgtggttgcaacggctaagcactacatcgcctacgagcaggagcatttccgtcaggcgcctgaagctcaaggctacggattcaatattaccgagagtggaagcgcgaacctcgacgataagactatgcatgagctgtacctctggcccttcgcggatgccatccgtgcaggtgccggtgctgtgatgtgctcgtacaaccagatcaacaacagctatggctgccagaacagctacactctgaacaagctgctcaaggctgagctgggtttccagggctttgtcatgagtgattgggcggctcaccatgccggtgtgagtggtgctttggcgggattggacatgtctatgccgggagacgtcgattacgacagtggcacgtcttactggggtaccaacttgaccatcagtgtgctcaacgggacggtgccccaatggcgtgttgatgacatggctgtccgcatcatggccgcctactacaaggtcggccgtgaccgtctgtggactcctcccaacttcagctcatggaccagagatgaatacggcttcaagtactactatgtctcggagggaccgtatgagaaggtcaaccagttcgtgaatgtgcaacgcaaccatagcgagttgatccgccgtattggagcagacagcacagtgctcctcaagaacgatggcgctcttcccttgactggaaaggagcgcttggtcgcccttatcggagaagatgcgggttccaatccttatggtgccaacggctgcagtgaccgtgggtgcgacaatggaacattggcgatgggctggggaagtggcactgccaactttccctacttggtgacccccgagcaggccatctcgaacgaggtgctcaagaacaagaatggcgtattcactgcgaccgataactgggctattgatcagattgaggcgcttgctaagaccgccaggtaagaagatctcgggctctttctttttccttattgtgcaatgaatgctgacaacatggtagtgtctctcttgtctttgtcaacgccgactctggtgagggttatatcaatgtcgacggaaacctgggtgaccgcaggaacctgaccctgtggaggaacggcgacaatgtgatcaaggctgctgctagcaactgcaacaacacgatcgttattattcactctgtcggcccagtcttggttaacgagtggtacgacaaccccaatgttaccgctattctctggggtggtcttcccggtcaggagtctggcaactccctcgccgacgtgctctacggccgtgtcaaccccggtgccaagtcgcccttcacctggggcaagactcgtgaggcctaccaagattacttgtacaccgagcccaacaacggcaacggagcgccccaggaagacttcgtcgagggcgtcttcattgactaccgcggatttgacaagcgcaacgagactcctatctatgagttcggctatggtctgagctacaccaccttcaactactcgaaccttcaggtggaggttctgagcgcccctgcgtacgagcctgcttcgggcgagactgaggcagcgccgactttcggagaggtcggaaatgcgtcggattacctctaccccgatggactgcagagaatcaccaagttcatctacccctggctcaacagtaccgatcttgaggcgtcttctggggatgctagctatgggcaggatgcctcagactatcttcccgagggagccaccgatggctctgcgcaaccgatcctgcctgccggtggtggtgctggcggcaaccctcgcctgtacgacgagctcatccgcgtgtcggtgactatcaagaacaccggcaaggttgcgggtgatgaagttcctcaactggtaagtagacagcaaattcgaacgaagtcggacaaagctaataaatcgcagtatgtttctcttggcggccctaacgaacccaagatcgtgctgcgtcaatttgagcgtatcacgctgcagccgtcggaagagacgcagtggagcacgactctgacgcgccgtgaccttgcgaactggaatgttgagacgcaggactgggagattacgtcgtatcccaagatggtgtttgtcggaagctcctcgcggaagctgccgctccgggcgtctctgcctactgttcactaa

SEQ ID NO: 5 - protein, 860 aa, Aspergillus niger
  SIGNAL 1..19
  PEPTIDE 20..860
MRFTLIEAVALTAVSLASADELAYSPPYYPSPWANGQGDWAEAYQRAVDIVSQMTLAEKVNLTTGTGWELELCVGQTGGVPRLGIPGMCAQDSPLGVRDSDYNSAFPAGVNVAATWDKNLAYLRGQAMGQEFSDKGADIQLGPAAGPLGRSPDGGRNWEGFSPDPALSGVLFAETIKGIQDAGVVATAKHYIAYEQEHFRQAPEAQGYGFNITESGSANLDDKTMHELYLWPFADAIRAGAGAVMCSYNQINNSYGCQNSYTLNKLLKAELGFQGFVMSDWAAHHAGVSGALAGLDMSMPGDVDYDSGTSYWGTNLTISVLNGTVPQWRVDDMAVRIMAAYYKVGRDRLWTPPNFSSWTRDEYGFKYYYVSEGPYEKVNQFVNVQRNHSELIRRIGADSTVLLKNDGALPLTGKERLVALIGEDAGSNPYGANGCSDRGCDNGTLAMGWGSGTANFPYLVTPEQAISNEVLKNKNGVFTATDNWAIDQIEALAKTASVSLVFVNADSGEGYINVDGNLGDRRNLTLWRNGDNVIKAAASNCNNTIVIIHSVGPVLVNEWYDNPNVTAILWGGLPGQESGNSLADVLYGRVNPGAKSPFTWGKTREAYQDYLYTEPNNGNGAPQEDFVEGVFIDYRGFDKRNETPIYEFGYGLSYTTFNYSNLQVEVLSAPAYEPASGETEAAPTFGEVGNASDYLYPDGLQRITKFIYPWLNSTDLEASSGDASYGQDASDYLPEGATDGSAQPILPAGGGAGGNPRLYDELIRVSVTIKNTGKVAGDEVPQLYVSLGGPNEPKIVLRQFERITLQPSEETQWSTTLTRRDLANWNVETQDWEITSYPKMVFVGSSSRKLPLRASLPTVH

SEQ ID NO: 6 - protein, 841 aa, Aspergillus niger
DELAYSPPYYPSPWANGQGDWAEAYQRAVDIVSQMTLAEKVNLTTGTGWELELCVGQTGGVPRLGIPGMCAQDSPLGVRDSDYNSAFPAGVNVAATWDKNLAYLRGQAMGQEFSDKGADIQLGPAAGPLGRSPDGGRNWEGFSPDPALSGVLFAETIKGIQDAGVVATAKHYIAYEQEHFRQAPEAQGYGFNITESGSANLDDKTMHELYLWPFADAIRAGAGAVMCSYNQINNSYGCQNSYTLNKLLKAELGFQGFVMSDWAAHHAGVSGALAGLDMSMPGDVDYDSGTSYWGTNLTISVLNGTVPQWRVDDMAVRIMAAYYKVGRDRLWTPPNFSSWTRDEYGFKYYYVSEGPYEKVNQFVNVQRNHSELIRRIGADSTVLLKNDGALPLTGKERLVALIGEDAGSNPYGANGCSDRGCDNGTLAMGWGSGTANFPYLVTPEQAISNEVLKNKNGVFTATDNWAIDQIEALAKTASVSLVFVNADSGEGYINVDGNLGDRRNLTLWRNGDNVIKAAASNCNNTIVIIHSVGPVLVNEWYDNPNVTAILWGGLPGQESGNSLADVLYGRVNPGAKSPFTWGKTREAYQDYLYTEPNNGNGAPQEDFVEGVFIDYRGFDKRNETPIYEFGYGLSYTTFNYSNLQVEVLSAPAYEPASGETEAAPTFGEVGNASDYLYPDGLQRITKFIYPWLNSTDLEASSGDASYGQDASDYLPEGATDGSAQPILPAGGGAGGNPRLYDELIRVSVTIKNTGKVAGDEVPQLYVSLGGPNEPKIVLRQFERITLQPSEETQWSTTLTRRDLANWNVETQDWEITSYPKMVFVGSSSRKLPLRASLPTVH

SEQ ID NO: 7 - DNA, 2168 bp, Aspergillus niger
  sig_peptide 1..54
  exon 55..214
  intron 215..285
  exon 286..572
  intron 573..627
  exon 628..724
  intron 725..785
  exon 786..1423
  intron 1424..1481
  exon 1482..2168
  source 1..2168: mol_type = genomic DNA
atgtcgttccgatctctactcgccctgagcggcctcgtctgcacagggttggcaaatgtgatttccaagcgcgcgaccttggattcatggttgagcaacgaagcgaccgtggctcgtactgccatcctgaataacatcggggcggacggtgcttgggtgtcgggcgcggactctggcattgtcgttgctagtcccagcacggataacccggactgtatgtttcgagctcagattatgagtgtgtcattgatcgattgatgctgactggcgtgtcgtttgttgtagacttctacacctggactcgcgactctggtctcgtcctcaaggccctcgtcgatctcttccgaaatggagataccagtctcctctccaccattgagaactacatctccgcccaggcaattgtccagggtatcagtaacccctctggtgatctgtccagcggcgctggtctcggtgaacccaagttcaatgtcgatgagactgcctacactggttcttggggacggccgcagcgagatggtccggctctgagagcaactgctatgatcgacttcgggcagtggctgcttgtatgttctccacccccttgcgtctgatctgtggcatatgtagctgactggttaggacaatggctacaccagcaccgcaacggacattgtttggcccctcgttaggaacgacctgtcgtatgtggctcaatactggaaccagacaggatatggtgtgtttgttctattttaaattttcaaagatgcgccagcagagctaacccgcgatcgcagatctctgggaagaagtcaatggctcgtctttcttcacgattgctgtgcaacaccgcgcccttgtcgaaggtagtgccttcgcgaagtccgtcggctcgtcctgctcctggtgtgattctcaggcacctgagattctctgctacctgcagtcattctggaccggcagcttcattttggccaacttcgatagcagccgttccggcaaggacgcaaacaccctcctgggaagcatccacacctttgatcctgaggccgcatgcgacgactccaccttccagccctgctccccccgcgcgctcgccaaccacaaggaggtcgtagactctttccgctcaatttataccctcaacgacggtctcagtgacagtgaggctgttgcggtcggtcggtaccctgaggacacgtactacaacggcaacccgtggttcctgtgcaccttggctgctgcagagcagttgtacgatgctctataccagtgggacaagcaggggtcgttggaggtcacggatgtgtcgctggacttcttcaaggcactgtacagcgatgctgctactggcacctactcttcgtccagctcgacttatagtagcattgtagatgccgtgaagactttcgccgatggcttcgtctctattgtggtaagtctacgctagacaagcgctcatgttgacagagggtgcgtactaacagaattaggaaactcacgccgcaagcaacggctccatgtccgagcaatacgataagtctgatggcgagcagctttccgctcgcgacctgacctggtcttatgctgctctgctgaccgccaacaaccgtcgtaactccgtcgtgcctgcttcttggggcgagacctctgccagcagcgtgcccggcacctgtgcggccacatctgccactggtacctacagcagtgtgactgtcacctcgtggcccagtatcgtggctactggcggcaccactacgacggctacccccactggatccggcagcgtgacctcgaccagcaagaccaccgcgactgctagcaagaccagcaccagtacgtcatcagcctcctgtaccactcccaccgccgtggctgtgactttcgatctgacagctaccaccacctacggcgagaacatctacctggtcggatcgatctctcagctgggtgactgggaaaccagcgacggcatagctctgagtgctgacaagtacacttccagcgacccgctctggtatgtcactgtgactctgccggctggtgagtcgtttgagtacaagtttatccgcattgagagcgatgactccgtggagtgggagagtgatcccaaccgagaatacaccgttcctcaggcgtgcggaacgtcgaccgcgacggtgactgacacctggcggtag

SEQ ID NO: 8 - protein, 640 aa, Aspergillus niger
  SIGNAL 1..18
  PEPTIDE 19..640
MSFRSLLALSGLVCTGLANVISKRATLDSWLSNEATVARTAILNNIGADGAWVSGADSGIVVASPSTDNPDYFYTWTRDSGLVLKALVDLFRNGDTSLLSTIENYISAQAIVQGISNPSGDLSSGAGLGEPKFNVDETAYTGSWGRPQRDGPALRATAMIDFGQWLLDNGYTSTATDIVWPLVRNDLSYVAQYWNQTGYDLWEEVNGSSFFTIAVQHRALVEGSAFAKSVGSSCSWCDSQAPEILCYLQSFWTGSFILANFDSSRSGKDANTLLGSIHTFDPEAACDDSTFQPCSPRALANHKEVVDSFRSIYTLNDGLSDSEAVAVGRYPEDTYYNGNPWFLCTLAAAEQLYDALYQWDKQGSLEVTDVSLDFFKALYSDAATGTYSSSSSTYSSIVDAVKTFADGFVSIVETHAASNGSMSEQYDKSDGEQLSARDLTWSYAALLTANNRRNSVVPASWGETSASSVPGTCAATSATGTYSSVTVTSWPSIVATGGTTTTATPTGSGSVTSTSKTTATASKTSTSTSSASCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 9 - protein, 622 aa, Aspergillus niger
NVISKRATLDSWLSNEATVARTAILNNIGADGAWVSGADSGIVVASPSTDNPDYFYTWTRDSGLVLKALVDLFRNGDTSLLSTIENYISAQAIVQGISNPSGDLSSGAGLGEPKFNVDETAYTGSWGRPQRDGPALRATAMIDFGQWLLDNGYTSTATDIVWPLVRNDLSYVAQYWNQTGYDLWEEVNGSSFFTIAVQHRALVEGSAFAKSVGSSCSWCDSQAPEILCYLQSFWTGSFILANFDSSRSGKDANTLLGSIHTFDPEAACDDSTFQPCSPRALANHKEVVDSFRSIYTLNDGLSDSEAVAVGRYPEDTYYNGNPWFLCTLAAAEQLYDALYQWDKQGSLEVTDVSLDFFKALYSDAATGTYSSSSSTYSSIVDAVKTFADGFVSIVETHAASNGSMSEQYDKSDGEQLSARDLTWSYAALLTANNRRNSVVPASWGETSASSVPGTCAATSATGTYSSVTVTSWPSIVATGGTTTTATPTGSGSVTSTSKTTATASKTSTSTSSASCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 10 - DNA, 1914 bp, Bipolaris sorokiniana
  source 1..1914: mol_type = genomic DNA
  exon 1..1914
atgaagttctcgtcgtcacgcgatttggtggtagtgttgtcgtttgcgatggaagcaacaagtcggattctggtccgtcaacctacgactattgactacaatgccgctccacccaatctctctaccctaacgaataactcgctcttcgaaacttggagacccaaagcacatgttctaccggcgtttggccagatcggagatccgtgtatgcattatacggatcctgccactgggcttttccatgtcggatatctgcatctgggggcatcgggcgcaacgacgagtgatttggttacgtacaaggacttgaatcccaattccgcgccgttcatccgtgcaggcgggataaacgaccccattgcagtcttcgacgggagtgtgattgaaaagggcgtaaacggcacgcctacgctcctctacacaagcgtcagctacctccccatccaatggacaatccgctacacaaaggggagcgaaacacaatcactagcctatgccacggacggcggcaaaaactttacaaaagccaagttcggccccgttatccctagccctcccttcgccgtcaacgtaacgggattcagagacccatacgtattccaaagcccacaatttgattctcttctcagcaagaaaaaagatacatggtacaccctcatctccggcggtgtccacggtgtaggccccagtctcttcctctacgcccaatacgacgactcagacgccgcctcattccaaacctgggagtatctaggcgaatactggcacgaagccgccaacaccacctggaccgaatccaactgggccggtcgctggggcttcaacttcgaagtcgcaaacatcttctccctcgacgagcacggcgcaaatccctccactggcgaaacctttttaaccgtaggcgcagaatggagctacgcacccatcgtgccccaagtgtccgacgaaagagatatgctctgggtcgctggcacccagtccctcatcgacggcaaactaacttttgaaccgacaatggcggggagattagatgcaggccgctccgcctacgcggcagccggcaagcctctctccgcagactcgcagcctagctgggcgagcggcgcccccgatcgcgcaatcacgtatctctggctcacgggcgatctctacggcacatccgccttccccacagcccagcaaaattggacgggctccctgcttctcccgcgtgagctatcgcgctgctacatcgacgtgctcgacacgccgctagcccgcgaaaccggggcgtggaggatcgagcgagagagcggagagggcacagtacggcttgcgacattgtgccagaagatcgcacgggaagttacgactgcgttcaaggcaaaggcaacaaacgtaatcacgttgcctgcagatgagctttctcctggcgagcaatgcaaactaccccagatccccaaggagaatcactacatgcttactgcctctctgacctttcccgcgcgcacaccgggtttgaaggcgggacttacgatcctctcgggcgcgcatgagacgactcgtgttgtgtacgatatcggctccgagcggctcagtgtggaacgtgggagttcgtctgcggcggcgaatacgacgcagggtatacctacgtatgatgaggagggtttgttccggctgttcgatttaccaagtcacgtcaatgcctcgcggattgagacactgaatctgacgattgtggtggatggtggtgtggtggaggtgcatgcgaatgatcggtttgctctgagtacgtgggtgaggccgtggtatgcggatagtaagggtgttggggttttcgtggagggagaaggagaggaggaggtacaggtgggcgaggtgaaggtttacgaggggttggtggatgcgtggccgaagaggaataagacgatttaa

SEQ ID NO: 11 - protein, 637 aa, Bipolaris sorokiniana
  SIGNAL 1..21
  PEPTIDE 22..637
MKFSSSRDLVVVLSFAMEATSRILVRQPTTIDYNAAPPNLSTLTNNSLFETWRPKAHVLPAFGQIGDPCMHYTDPATGLFHVGYLHLGASGATTSDLVTYKDLNPNSAPFIRAGGINDPIAVFDGSVIEKGVNGTPTLLYTSVSYLPIQWTIRYTKGSETQSLAYATDGGKNFTKAKFGPVIPSPPFAVNVTGFRDPYVFQSPQFDSLLSKKKDTWYTLISGGVHGVGPSLFLYAQYDDSDAASFQTWEYLGEYWHEAANTTWTESNWAGRWGFNFEVANIFSLDEHGANPSTGETFLTVGAEWSYAPIVPQVSDERDMLWVAGTQSLIDGKLTFEPTMAGRLDAGRSAYAAAGKPLSADSQPSWASGAPDRAITYLWLTGDLYGTSAFPTAQQNWTGSLLLPRELSRCYIDVLDTPLARETGAWRIERESGEGTVRLATLCQKIAREVTTAFKAKATNVITLPADELSPGEQCKLPQIPKENHYMLTASLTFPARTPGLKAGLTILSGAHETTRVVYDIGSERLSVERGSSSAAANTTQGIPTYDEEGLFRLFDLPSHVNASRIETLNLTIVVDGGVVEVHANDRFALSTWVRPWYADSKGVGVFVEGEGEEEVQVGEVKVYEGLVDAWPKRNKTI

SEQ ID NO: 12 - protein, 616 aa, Bipolaris sorokiniana
RILVRQPTTIDYNAAPPNLSTLTNNSLFETWRPKAHVLPAFGQIGDPCMHYTDPATGLFHVGYLHLGASGATTSDLVTYKDLNPNSAPFIRAGGINDPIAVFDGSVIEKGVNGTPTLLYTSVSYLPIQWTIRYTKGSETQSLAYATDGGKNFTKAKFGPVIPSPPFAVNVTGFRDPYVFQSPQFDSLLSKKKDTWYTLISGGVHGVGPSLFLYAQYDDSDAASFQTWEYLGEYWHEAANTTWTESNWAGRWGFNFEVANIFSLDEHGANPSTGETFLTVGAEWSYAPIVPQVSDERDMLWVAGTQSLIDGKLTFEPTMAGRLDAGRSAYAAAGKPLSADSQPSWASGAPDRAITYLWLTGDLYGTSAFPTAQQNWTGSLLLPRELSRCYIDVLDTPLARETGAWRIERESGEGTVRLATLCQKIAREVTTAFKAKATNVITLPADELSPGEQCKLPQIPKENHYMLTASLTFPARTPGLKAGLTILSGAHETTRVVYDIGSERLSVERGSSSAAANTTQGIPTYDEEGLFRLFDLPSHVNASRIETLNLTIVVDGGVVEVHANDRFALSTWVRPWYADSKGVGVFVEGEGEEEVQVGEVKVYEGLVDAWPKRNKTI

SEQ ID NO: 13 - DNA, 1956 bp, Aspergillus aculeatus
  source 1..1956: mol_type = genomic DNA
  exon 1..1956
atgaagctcaccactaccacgctggcgctcgccaccggcgcagcagcctcatatcacctggacaccacggccccgccgccgaccaacctcagcaccctccccaacaacaccctcttccacctgtggcggccgcgcgcgcacatcctgcccgccgagggccagatcggcgacccctgcgcgcactacaccgacccatccaccggcctcttccacgtggggttcctgcacgacggggacggcatcgcgggcgccaccacggccaacctggccacctacaccgacacctccgataacgggagcttcctgatccagccgggcgggaagaacgaccccgtcgccgtgttcgacggcgccgtcatccccgtcggcgtcaacaacacccccaccttactctacacctccgtctccttcctgcccatccactggtccatcccctacacccgcggcagcgagacgcagtcgttggccgtcgcgcgcgacggcggccaccgcttcgacaagctcgaccagggccccgtcatcgccgaccaccccttcgccgtcgacgtcaccgccttccgcgatccgtttgtcttccgcagtgccaggttggatgtgctgctgtcgttggatgaggaggtggcgcggaatgagacggccgtgcagcaggccgtcgatggctggaccgagaagaacgccccctggtatgtcgcggtctctggcggggtgcacggcgtcgggcccgcgcagttcctctaccgccagaacggcgggaacgcttccgagttccagtactgggagtacctcggggagtggtggcaggaggcgaccaactccagctggggcgacgagggcacctgggccgggcgctgggggttcaacttcgagacggggaatgtgctcttcctcaccgaggagggccatgacccccagacgggcgaggtgttcgtcaccctcggcacggaggggtctggcctgcccatcgtgccgcaggtctccagtatccacgatatgctgtgggcggcgggtgaggtcggggtgggcagtgagcaggagggtgccaaggtcgagttctccccctccatggccgggtttctggactgggggttcagcgcctacgctgcggcgggcaaggtgctgccggccagctcggcggtgtcgaagaccagcggcgtggaggtggatcggtatgtctcgttcgtctggttgacgggcgaccagtacgagcaggcggacgggttccccacggcccagcaggggtggacggggtcgctgctgctgccgcgcgagctgaaggtgcagacggtggagaacgtcgtcgacaacgagctggtgcgcgaggagggcgtgtcgtgggtggtgggggagtcggacaaccagacggccacgctgcgcacgctggggatcacgatcgcccgggagaccaaggcggccctgctggccaacggctcggtgaccgcggaggaggaccgcacgctgcagacggcggccgtcgtgccgttcgcgcaatcgccgagctccaagttcttcgtgctgacggcccagctggagttccccgcgaacgcgcgctcgtccccgctccagtccgggttcgaaatcctggcgtcggagctggagcgcacggccgtctactaccagttcagcaatgagtcgctggtcgtcgaccgcagtcagaccagtgcggcagcgcccacgaaccccgggctggatagctttactgagtccggcaagttgcggttgttcgacgtcatcgagaacggccaggagcaggtcgagacgttggatctcactgtcgtcgtggataactcggttgtcgaggtgtatgccaacgggcgctttgcgttgagcacctgggcgagatcgtggtacgacaactccacccagatccgcttcttccacaacggcgagggcgaggtgcagttcaggaatgtttccgtgtcggaggggctctataacgcctggccggagagaaattaa

SEQ ID NO: 14 - protein, 651 aa, Aspergillus aculeatus
  SIGNAL 1..16
  PEPTIDE 17..651
MKLTTTTLALATGAAASYHLDTTAPPPTNLSTLPNNTLFHLWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGHRFDKLDQGPVIADHPFAVDVTAFRDPFVFRSARLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTATLRTLGITIARETKAALLANGSVTAEEDRTLQTAAVVPFAQSPSSKFFVLTAQLEFPANARSSPLQSGFEILASELERTAVYYQFSNESLVVDRSQTSAAAPTNPGLDSFTESGKLRLFDVIENGQEQVETLDLTVVVDNSVVEVYANGRFALSTWARSWYDNSTQIRFFHNGEGEVQFRNVSVSEGLYNAWPERN

SEQ ID NO: 15 - protein, 635 aa, Aspergillus aculeatus
SYHLDTTAPPPTNLSTLPNNTLFHLWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGHRFDKLDQGPVIADHPFAVDVTAFRDPFVFRSARLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTATLRTLGITIARETKAALLANGSVTAEEDRTLQTAAVVPFAQSPSSKFFVLTAQLEFPANARSSPLQSGFEILASELERTAVYYQFSNESLVVDRSQTSAAAPTNPGLDSFTESGKLRLFDVIENGQEQVETLDLTVVVDNSVVEVYANGRFALSTWARSWYDNSTQIRFFHNGEGEVQFRNVSVSEGLYNAWPERN

SEQ ID NO: 16 - DNA, 2020 bp, Pestalotiopsis vismiae
  source 1..2020: mol_type = genomic DNA
  exon 1..180
  intron 181..257
  exon 258..1822
  intron 1823..1899
  exon 1900..2020
atgtggtccctcgtcttcttcgctgccggagcactcggcgggctgtcccgtcacaagcgggcagtcatagactacaactcggcgcctcccaacctgtccacgctagccaacggttctctgttcgagacgtggcggcctcgcgctcacatcctgcctccttttggacaaatcggagatccagtaagcagacatcacaacttacctatttattggttctgatttttgcttctcaaaatgtattcgctaacacagattagtgcgcccactacgtggatgccgagacgggcactgtccatttcggctggcttcacgatggcgcttcgggcgcgaccacggatgatcttgtcacatataaggacctgaatgcgaatggcgcccccttcatcgaagccggaggactcaacgacccggtcgccgtatttgacggtaccgtaatcaacgtaggcgtgggaggaaaaccgacatttctctacacttcagtctcgtatctgcccatccagtggaccattgcgtatacccgcggaagcgagacgcagtccctggctgtgagctacgacgatggcaggaactttaccaagctcgagcagggcccggtcattccaggacctcctttcgccgtcaacgtcactggattccgggatccttacgcctttcagagcacgcagctcgacacgcttctcaacggtgccaatggtacgtggtaccttgtcatctcgggaggcgtacacggtgacggaccgagccaattcctctaccgccagtatgatccggaattccagtattgggaggagctcggccagtggtggcacgaggcggctaacacgacgtggggcaacggcgattgggcgggccgctggggcttcaattttgaggtatcaaacatctttacgctggatgaggacggatacagcgacgagggagagttcttcaccaccatgggagccgagtggtcgctggcacccatcgtacctcaagtctctgatttcagggacgctctgtgggtctcggggaatgttaccatcgaggatggcgaggtcaaattcagcccttcgtatgctggaaagctggaatggggacactcggcgtatgcgacccacggacaggttgtccgagcagactcgaagccgtccatggcaagcggagctcctgaccggttcatttccaccacgtggctcactggagactattacggcacgctggacccggctaccaatcaacagaactggaccggcgcacttcttctgccacgagagctcagcattggtaagatttgcaacgtggtagacaacgatctcgcgcgagagatcggttcgtggaagacgagtcgcaacgactccgggattgtggacctttctaccctcaaacagacgattgcgagagagtcgcttgccgcccttttgagaagcaacgagacattgcaggaagagagtcgcacactcaattcttccgccgtggtgccattccaacaatctcccgcttcaaagttctttgtcctagaagcctcgatctccgtgccacaatcagctcgagacagtgatctcaaggcaggattccagatacttgcctccgaatacgagagcacgactatctactaccaattcagcaacgagtcggtcatcgtggaccgaaccaacaccagcgctgccgcaaagactaccaatgacattgactcccggaatgaagctgggcgactccgactattcgatattgcgcaggaagacggggaggcagcagtagaggtcctcaaaatgaccatcgtggtcgataacgcagtggttgaaatccacgccaacgatcggttcgctttgagcacatgggcgcggtaagttctcggaaatctcacttcatctgatacgtttctgggagaatttacattttctaacttgggaatatttgtagctcttggtacggctcatcaactgagattcgcttctttcaggaaggagctggtcaagtaacgtttgagaatattacaattcacgaaggcctgtttgacgcctggccagaacgacaaaactaa

SEQ ID NO: 17 - protein, 621 aa, Pestalotiopsis vismiae
  SIGNAL 1..20
  PEPTIDE 21..621
MWSLVFFAAGALGGLSRHKRAVIDYNSAPPNLSTLANGSLFETWRPRAHILPPFGQIGDPCAHYVDAETGTVHFGWLHDGASGATTDDLVTYKDLNANGAPFIEAGGLNDPVAVFDGTVINVGVGGKPTFLYTSVSYLPIQWTIAYTRGSETQSLAVSYDDGRNFTKLEQGPVIPGPPFAVNVTGFRDPYAFQSTQLDTLLNGANGTWYLVISGGVHGDGPSQFLYRQYDPEFQYWEELGQWWHEAANTTWGNGDWAGRWGFNFEVSNIFTLDEDGYSDEGEFFTTMGAEWSLAPIVPQVSDFRDALWVSGNVTIEDGEVKFSPSYAGKLEWGHSAYATHGQVVRADSKPSMASGAPDRFISTTWLTGDYYGTLDPATNQQNWTGALLLPRELSIGKICNVVDNDLAREIGSWKTSRNDSGIVDLSTLKQTIARESLAALLRSNETLQEESRTLNSSAVVPFQQSPASKFFVLEASISVPQSARDSDLKAGFQILASEYESTTIYYQFSNESVIVDRTNTSAAAKTTNDIDSRNEAGRLRLFDIAQEDGEAAVEVLKMTIVVDNAVVEIHANDRFALSTWARSWYGSSTEIRFFQEGAGQVTFENITIHEGLFDAWPERQN

SEQ ID NO: 18 - protein, 601 aa, Pestalotiopsis vismiae
AVIDYNSAPPNLSTLANGSLFETWRPRAHILPPFGQIGDPCAHYVDAETGTVHFGWLHDGASGATTDDLVTYKDLNANGAPFIEAGGLNDPVAVFDGTVINVGVGGKPTFLYTSVSYLPIQWTIAYTRGSETQSLAVSYDDGRNFTKLEQGPVIPGPPFAVNVTGFRDPYAFQSTQLDTLLNGANGTWYLVISGGVHGDGPSQFLYRQYDPEFQYWEELGQWWHEAANTTWGNGDWAGRWGFNFEVSNIFTLDEDGYSDEGEFFTTMGAEWSLAPIVPQVSDFRDALWVSGNVTIEDGEVKFSPSYAGKLEWGHSAYATHGQVVRADSKPSMASGAPDRFISTTWLTGDYYGTLDPATNQQNWTGALLLPRELSIGKICNVVDNDLAREIGSWKTSRNDSGIVDLSTLKQTIARESLAALLRSNETLQEESRTLNSSAVVPFQQSPASKFFVLEASISVPQSARDSDLKAGFQILASEYESTTIYYQFSNESVIVDRTNTSAAAKTTNDIDSRNEAGRLRLFDIAQEDGEAAVEVLKMTIVVDNAVVEIHANDRFALSTWARSWYGSSTEIRFFQEGAGQVTFENITIHEGLFDAWPERQN

SEQ ID NO: 19 - DNA, 1925 bp, Aspergillus avenaceus
  source 1..1925: mol_type = genomic DNA
  exon 1..1757
  intron 1758..1807
  exon 1808..1925
atgaagctctcctccaccgcggtgcctctggtcaccagccaggcagcttacgctgcgtcggctatcgattacaatgcagcccccccaaacctatcgactctggccaatggttctttgtatgagacatggagaccccgcgctcatcttctgccacccacgggtcgcattggcgacccatgcgcccattataccgaccccagcactggtgcgttccatttaggctggctgtataatggcacgggtatctcgggggctaccaccgatgatttggtgaaataccgagacctgaaccccaacggcggacaaatcattgttcccggtgggaaaaacgatcctttagctgtgtttgatggaacggtgttcccgaagggcgtggatggcaagcctaccctcatttatacctccgtcagctcacttcccattcattggtcgattccttataaccctggcgccgagagtcagtccttggctgtcagcaatgatggcagcggccgcaacttcaccaagctagaccagccgccggtgatccccttgcctccatcggacctggatgttactgcgttccgcgacccctacgcgttccagagccaagaattggacacggccgcgaaaagcaatgcaggcacctggtacacggttatctctggcggagtccacgagaaaggccctggccagttcctttaccgccagagccaggcgaacatgagcctagacagctgggagtacctcggccagtggtggcatgagagcgtcaatggaacctggggaaatggcgactgggccggaggatggggtttcaattttgagactggaaacatcttcagcctgaacgaggagggatacagcactgacggtgagatgttcatgactctgggtactgagggatcgggcactcctattgtgtcccaggtgtcttctatccacgatatgctgtgggtggccggcaatgtttcaaacaacggcaacatcactttcacccccagcatggcgggtatcttcgactggggtgcttctggttacgctgcatctggtaacatcctttccgccagctccaaggcctctaccaagagcggtgcccccgaccgcttcatctccaccgtctggttgactggcgatttgttcgagcaggctaagggatacccggcagcacaacaaggctgggttggtactcttcttctgcctcgtgagctgcggatcaagaccatctccggtgtggccgacaacgagcttgcccgtgaaaccggctcatcctggcgcgtgaggaacagcgaatccgccgccggttatctcgagctggaaaccctgggcattgacatcgcccgtgagacccgcgaggccttgttctccggctcgaagattagcgagcaagagcgcaccctcactgaagcaggtggcgtccccttccaggtctcccccacgaccaagtactacgctctcagcgctaagctgtccttccctcgctcggcccggaactccgacctccaggctggattccaggtgctgtcttccaaccttgaaagcaccactatctactaccagttctccaacgaatcgatcattgtcgatcgcagcaacaccagcgccgcttccaagaccaccgatggtattgtcagtgcgaacgaggctggccgtctccgtttgttcgacctgaagggtgagaccgagaagatcgagactctggacttgaccattgttgtcgacaacgcagtcgtggaagtttatgccaatggccgctttgccttgggtacctgggctcggtatgtattgtgtcaaatgcagaaagcaggcataggctaactagtaataggtcatggtaccagaactcgaccgacatcaagttcttccacaacggtgttggcgaagtgactttcagcgacgtcactgtttccgaggggctgtttgaggcttggccggagcgtgcttaa

SEQ ID NO: 20 - protein, 624 aa, Aspergillus avenaceus
  SIGNAL 1..18
  PEPTIDE 19..624
MKLSSTAVPLVTSQAAYAASAIDYNAAPPNLSTLANGSLYETWRPRAHLLPPTGRIGDPCAHYTDPSTGAFHLGWLYNGTGISGATTDDLVKYRDLNPNGGQIIVPGGKNDPLAVFDGTVFPKGVDGKPTLIYTSVSSLPIHWSIPYNPGAESQSLAVSNDGSGRNFTKLDQPPVIPLPPSDLDVTAFRDPYAFQSQELDTAAKSNAGTWYTVISGGVHEKGPGQFLYRQSQANMSLDSWEYLGQWWHESVNGTWGNGDWAGGWGFNFETGNIFSLNEEGYSTDGEMFMTLGTEGSGTPIVSQVSSIHDMLWVAGNVSNNGNITFTPSMAGIFDWGASGYAASGNILSASSKASTKSGAPDRFISTVWLTGDLFEQAKGYPAAQQGWVGTLLLPRELRIKTISGVADNELARETGSSWRVRNSESAAGYLELETLGIDIARETREALFSGSKISEQERTLTEAGGVPFQVSPTTKYYALSAKLSFPRSARNSDLQAGFQVLSSNLESTTIYYQFSNESIIVDRSNTSAASKTTDGIVSANEAGRLRLFDLKGETEKIETLDLTIVVDNAVVEVYANGRFALGTWARSWYQNSTDIKFFHNGVGEVTFSDVTVSEGLFEAWPERA

SEQ ID NO: 21 - protein, 606 aa, Aspergillus avenaceus
ASAIDYNAAPPNLSTLANGSLYETWRPRAHLLPPTGRIGDPCAHYTDPSTGAFHLGWLYNGTGISGATTDDLVKYRDLNPNGGQIIVPGGKNDPLAVFDGTVFPKGVDGKPTLIYTSVSSLPIHWSIPYNPGAESQSLAVSNDGSGRNFTKLDQPPVIPLPPSDLDVTAFRDPYAFQSQELDTAAKSNAGTWYTVISGGVHEKGPGQFLYRQSQANMSLDSWEYLGQWWHESVNGTWGNGDWAGGWGFNFETGNIFSLNEEGYSTDGEMFMTLGTEGSGTPIVSQVSSIHDMLWVAGNVSNNGNITFTPSMAGIFDWGASGYAASGNILSASSKASTKSGAPDRFISTVWLTGDLFEQAKGYPAAQQGWVGTLLLPRELRIKTISGVADNELARETGSSWRVRNSESAAGYLELETLGIDIARETREALFSGSKISEQERTLTEAGGVPFQVSPTTKYYALSAKLSFPRSARNSDLQAGFQVLSSNLESTTIYYQFSNESIIVDRSNTSAASKTTDGIVSANEAGRLRLFDLKGETEKIETLDLTIVVDNAVVEVYANGRFALGTWARSWYQNSTDIKFFHNGVGEVTFSDVTVSEGLFEAWPERA

SEQ ID NO: 22 - DNA, 1922 bp, Aspergillus sclerotiorum
  source 1..1922: mol_type = genomic DNA
  exon 1..1748
  intron 1749..1804
  exon 1805..1922
atgaagccctcgccagccgcgcttctccttgccgccagccaggtcgccgaagctgttgattacaacgcggcgccaccgaacctgtcaaccctggccaacggctccctatacgagacatggagacctcgcgcccacgtgctgccgccgtcgggcaaaatcggtgatccctgcatgcagtacacagatcccaagacgggtctgttccacgttggctggctgcacaatggaacgggcgtttcaggcgccacgaccgacgatatggcgacctatcgcgatctgaaccccggtggcgggccggccattgtccctggcggtatcaatgaccctcttgctgtcttcgacggctccgtcatccccagtggcatcaacggcacccccacattcctgtatacatcggtctcgttcctccccatccactggtccatcccgtacacccggggtgcggagactcagtccctggctgtctcgtatgacggcggccgcaacttcaccaaagttcctcagggccctgtcatccctgatgcgccgttcggcttgaacgtgacggcctttcgcgatccatacgtcttccagaacccgaagctggatgcctccctcgacagcaccccgggaacctggtacactgtgatctccggtggcgtgcacaacgtcgggcccagccagttcctgtaccgccagaccggcccggacttccagtactgggagtacctgggagaatggtggcgcgagggcgtcaacacgacctggggtgacggcacctgggccggcggctggggattcaacttcgaagtggccaatgtgttcagtctgaacgacgagggatacgatcccgagggcgagatcttcaccaccattggaaccgagggctcctcgatgcccatcaagcgccgggtcagctctatccacgacatgctttggatctccggtcagatctctaataacggatccgtcaaattcaccccgactatggccggtgtcctggactggggtgcctccgcgtacgcggcagccggcaaaatcgtcacggccgactcgcaagcgtcgaagaagagcggcgccccagagcggttcatcacttacatctggctcacaggcgatgagtacgaactcgtcgaaggattccccaccaggcaacagaactggaccggaaccctccttctccctcgcgaacttcacaccctccgcatccctaacgtcgttgacaacgatatcgtccgggaacccagctcgtcctggcgcgtggagaagaagagctcatcaaagtctagaaccgtggaactcaagaccctaggcatagacatcgcgcgtgaaacctacgacgctcttctctctggcccttcgactgaagagcccgcccgcaaactcaccaaccagtccacgcctattcctttcgcccactccctagagagcaagttcttcgtcctacgcgccaccctcacattcccgaaatcggcgcgtacatcaggcctcaagagtggattccagatcctctcctctgaccacgagcacaccaacatctactaccagttctccaacgaatcgatcatcatcgatagaacccacaccagcgccgctgcggaatctacacttggaatcaccagctacaatgaggcggggcgtctccgtctcttcgatgttatcgatggtggcgccgagaagatcgagtcgttggatttgacgattgtcgtggataacggcgttttggaagtgtttgccaacggtcggttcgcgatgagtacatgggctcggtaagttctagttttggaccttgaagagaaacagacagtggctgattttattacaggtcttggtatgccaactctactgacatcaaattccttcacaatggcgttggcgaggtgcaattcagcaaggtgtcggtgtctgaggggctgtttgatgcttggccggagaggaactaa

SEQ ID NO: 23 - protein, 621 aa, Aspergillus sclerotiorum
  SIGNAL 1..18
  PEPTIDE 19..621
MKPSPAALLLAASQVAEAVDYNAAPPNLSTLANGSLYETWRPRAHVLPPSGKIGDPCMQYTDPKTGLFHVGWLHNGTGVSGATTDDMATYRDLNPGGGPAIVPGGINDPLAVFDGSVIPSGINGTPTFLYTSVSFLPIHWSIPYTRGAETQSLAVSYDGGRNFTKVPQGPVIPDAPFGLNVTAFRDPYVFQNPKLDASLDSTPGTWYTVISGGVHNVGPSQFLYRQTGPDFQYWEYLGEWWREGVNTTWGDGTWAGGWGFNFEVANVFSLNDEGYDPEGEIFTTIGTEGSSMPIKRRVSSIHDMLWISGQISNNGSVKFTPTMAGVLDWGASAYAAAGKIVTADSQASKKSGAPERFITYIWLTGDEYELVEGFPTRQQNWTGTLLLPRELHTLRIPNVVDNDIVREPSSSWRVEKKSSSKSRTVELKTLGIDIARETYDALLSGPSTEEPARKLTNQSTPIPFAHSLESKFFVLRATLTFPKSARTSGLKSGFQILSSDHEHTNIYYQFSNESIIIDRTHTSAAAESTLGITSYNEAGRLRLFDVIDGGAEKIESLDLTIVVDNGVLEVFANGRFAMSTWARSWYANSTDIKFLHNGVGEVQFSKVSVSEGLFDAWPERN

SEQ ID NO: 24 - protein, 603 aa, Aspergillus sclerotiorum
VDYNAAPPNLSTLANGSLYETWRPRAHVLPPSGKIGDPCMQYTDPKTGLFHVGWLHNGTGVSGATTDDMATYRDLNPGGGPAIVPGGINDPLAVFDGSVIPSGINGTPTFLYTSVSFLPIHWSIPYTRGAETQSLAVSYDGGRNFTKVPQGPVIPDAPFGLNVTAFRDPYVFQNPKLDASLDSTPGTWYTVISGGVHNVGPSQFLYRQTGPDFQYWEYLGEWWREGVNTTWGDGTWAGGWGFNFEVANVFSLNDEGYDPEGEIFTTIGTEGSSMPIKRRVSSIHDMLWISGQISNNGSVKFTPTMAGVLDWGASAYAAAGKIVTADSQASKKSGAPERFITYIWLTGDEYELVEGFPTRQQNWTGTLLLPRELHTLRIPNVVDNDIVREPSSSWRVEKKSSSKSRTVELKTLGIDIARETYDALLSGPSTEEPARKLTNQSTPIPFAHSLESKFFVLRATLTFPKSARTSGLKSGFQILSSDHEHTNIYYQFSNESIIIDRTHTSAAAESTLGITSYNEAGRLRLFDVIDGGAEKIESLDLTIVVDNGVLEVFANGRFAMSTWARSWYANSTDIKFLHNGVGEVQFSKVSVSEGLFDAWPERN

SEQ ID NO: 25 - DNA, 1910 bp, Fusarium avenaceum
  source 1..1910: mol_type = genomic DNA
  exon 1..1739
  intron 1740..1789
  exon 1790..1910
atgaagctctcacttttcttaacggttggctcttttgcctgggccagtccctcagctcccaagctagactacaacaaagctcccccgaatctatcgacgctagccaatctcacaatatacaacacatggcgtcctcgagctcacgtgcttccaccctctggacagatcggagacccttgtatgcattacactgatcccaagactggactcttccacgttggataccttcatgagggtgccgctggtgccaccacggatgatctggtgacataccatgatcttaacccaggtggtgctcctttcattcgagctggaggcctgaacgatccagtggctgtctttgatggctctgttattccaagcggtatcaatggcaccccgacgctcttctacacctcggtctcttatcttcccatccattggacaatcaactacacccgaggtgctgaaacacaatcattggctgtatcaacagatggaggccgcaacttcacaaaacttcaacaaggacctgctatcccggcgccgccctttgcgcttaacatgaccgctttccgcgaccctttcgtcttccagaacaaggagcttgatgcccttctgaagagcgagtctggaacatggtacaccaccatctgtggcggtgtgcaccaaaagggccccagtatgttcctctaccgacagcacgaccccgagttccagtactgggagtatctcggcgagtggtggcacgagaaggccaacaccacctggggcgatggtatctgggctggacgctacggttataacttcgaggttggcaacatcttcagcatcgacgaggagggatacaactcagacggcgagacttttgtcacctttggagccgaatggggtgaggcgcctatcgttcctcagaagtcccctgagcgtgacatgctctgggctgcaggcaagacgaccctagaagacggcaaggccaagtttactccctcaatggtcggaaaatacgactggggacgctctgcttacgctgccgctggcaagtatctgccttccgattccaaggcttcatccaagagcaaggccccagaccgattcatcagttacctgtggctcactggagattactacggtgatctcaagctgttccccacccgacagcagaattggaccggatctcttctacttccccgagagcttaccgtcggcaagatcagcaatgtcgtcgataacgagctttctcgcgaagagggctcgtggcgcgctgagaagaacgagtcaggtatcgtggagctcgctactatgaagcaggtcatcgcccgagaggcaatttcagcctttaccaagacgaaggccctcgtcgagcctggccgaaatatcagcaagtctggctcgatcgcatttgatcgcagccccaagtccaagttttacattctcaagagctccgtcacattccccaagtcagcccgcaactcggatctcaaggctggcttccagattcttgcgtccgagaaagagtctacgaccatttactaccaattctccaacgaatccatggtcatcgatcgctccaacaccagtgctgccgcgcttaccaccagcgacatcgactctaaaaacgaatcaggtcgcctccgtctatttgacgtcgtggctgacgatgaggagcagattgagactctcgaccttaccattgttgtcgacaacgctatagtagaggtccatgccaatgatcgctttggaatttccacctgggctcggtaagtttacttttctccgagattggatttatttactgaccgaattttagttcttggtatgccgattcgactgatattcgcttcttccacagcggaagtggtgaggtaactttcagtaatgtgacgattcatgagggtctcgcagaggcttggccggagaggaagcgataa

SEQ ID NO: 26 - protein, 619 aa, Fusarium avenaceum
  SIGNAL 1..15
  PEPTIDE 16..619
MKLSLFLTVGSFAWASPSAPKLDYNKAPPNLSTLANLTIYNTWRPRAHVLPPSGQIGDPCMHYTDPKTGLFHVGYLHEGAAGATTDDLVTYHDLNPGGAPFIRAGGLNDPVAVFDGSVIPSGINGTPTLFYTSVSYLPIHWTINYTRGAETQSLAVSTDGGRNFTKLQQGPAIPAPPFALNMTAFRDPFVFQNKELDALLKSESGTWYTTICGGVHQKGPSMFLYRQHDPEFQYWEYLGEWWHEKANTTWGDGIWAGRYGYNFEVGNIFSIDEEGYNSDGETFVTFGAEWGEAPIVPQKSPERDMLWAAGKTTLEDGKAKFTPSMVGKYDWGRSAYAAAGKYLPSDSKASSKSKAPDRFISYLWLTGDYYGDLKLFPTRQQNWTGSLLLPRELTVGKISNVVDNELSREEGSWRAEKNESGIVELATMKQVIAREAISAFTKTKALVEPGRNISKSGSIAFDRSPKSKFYILKSSVTFPKSARNSDLKAGFQILASEKESTTIYYQFSNESMVIDRSNTSAAALTTSDIDSKNESGRLRLFDVVADDEEQIETLDLTIVVDNAIVEVHANDRFGISTWARSWYADSTDIRFFHSGSGEVTFSNVTIHEGLAEAWPERKR

SEQ ID NO: 27 - protein, 604 aa, Fusarium avenaceum
SPSAPKLDYNKAPPNLSTLANLTIYNTWRPRAHVLPPSGQIGDPCMHYTDPKTGLFHVGYLHEGAAGATTDDLVTYHDLNPGGAPFIRAGGLNDPVAVFDGSVIPSGINGTPTLFYTSVSYLPIHWTINYTRGAETQSLAVSTDGGRNFTKLQQGPAIPAPPFALNMTAFRDPFVFQNKELDALLKSESGTWYTTICGGVHQKGPSMFLYRQHDPEFQYWEYLGEWWHEKANTTWGDGIWAGRYGYNFEVGNIFSIDEEGYNSDGETFVTFGAEWGEAPIVPQKSPERDMLWAAGKTTLEDGKAKFTPSMVGKYDWGRSAYAAAGKYLPSDSKASSKSKAPDRFISYLWLTGDYYGDLKLFPTRQQNWTGSLLLPRELTVGKISNVVDNELSREEGSWRAEKNESGIVELATMKQVIAREAISAFTKTKALVEPGRNISKSGSIAFDRSPKSKFYILKSSVTFPKSARNSDLKAGFQILASEKESTTIYYQFSNESMVIDRSNTSAAALTTSDIDSKNESGRLRLFDVVADDEEQIETLDLTIVVDNAIVEVHANDRFGISTWARSWYADSTDIRFFHSGSGEVTFSNVTIHEGLAEAWPERKR

SEQ ID NO: 28 - DNA, 1913 bp, Penicillium coprophilum
  source 1..1913: mol_type = genomic DNA
  exon 1..1745
  intron 1746..1795
  exon 1796..1913
atgaaactgtcaagtgcaatcaccgtggttttggcgaatcgagcttttgctgttgtttataaatctcaaattgactataactcagctcctccgaacctttctactttgacaaatgcatcccttttcgagacctggagacccagagcccatatcctccctccatctgggaagataggtgacccgtgcgggcaatacaccgatcctaagactggtctcttccatgttggttggctccatgaaggaattgcaggggctacaaccgatgatctcgctacttacagagacctcaatcctgatggagccccgtcgattattgcgggaggaaagaatgacccccttgctgttttcgatggctctgtcattccaagcggcatagacggcatgccaactctgctatatacctctgtgtcataccttccaattcattggtccatcccctacacccggggaagtgagacacagtcattggccgtttcctatgatggtggacgcaatttcaccaagcttgaccagggcccgatgatcccttcgcctccttttgctgtcaatgtcaccgctttccgtgacccctacgctttccagagcccagcccttgacatagctgtcaacagtactcgaggaacttggtatactgccatctcaggtggggtccacgatgtcggaccctgtcagtttctctaccggcagcatgatgcggacttccaatattgggaatatctcggtcaatggtggagtgagcctgtcaatacaacttggggaaatggcgactgggcgggtggatggggcttcaactttgaggttggaaatgtcttcagcctgaatgcagacggttacagcgaggacggcgaagtcttcatgacccttggtaccgagagttctgaagttcccatcgttcccaaggtatcttcaattcacgatatggtgtgggtagccgggaatattacaaacgatggctccgtcaccttcaacccaaccatggcaggtgtgcttgactggggcttgtcggcatatgctgctgccggcaaaattttgccagcgagctctcaggcatctaggaagagcggtgctcctgatcgatttgtctcctacacctggctcactggagatttatatgagctatcgaaaggattcccttccgctcagcaaaactggacaggtactctcttgctgccgcgggagctgaatgttcgcactatccgtaacgtggtagacaacaaactgtcgcgtgagtctttggcatcgtggcgtgtggcccgcgagggacacggtcaaattgacctcgaaacgatgggaatctcaattgccagggagacctacagcgctctcacatccggctcatcttctgtggagtcggatcaaacattatcggaggctgggtcaaaggcattcagcatctcgccctcaaccaagttctttgttctgacagcgaatatctctttccctacctctgcccgtgactcgggtattcaatctggcttccagattttgtcctccgatcttgaatctacgacgatctattaccaattttcaaacgagtccatcatcgtggaccgcagtaacacaagtgccgcgtcgaaaacgactgatggaattagcagtgccaatgaggcgggccgtctacggctatttgatgtcttgcaaaatggaaaaaagcagattgagacattggaactcaccgtgattgtggataatggggttcttgaaatatatgccaatggacggtttgctctaagcacatgggctcggtacgtttatcttatttctaaaatatgggacgatatctaactacatgaaggtcttggtacgccaactcgacgggaattaacttcttccataacggcataggagaaacgacatttggagatgtgaaggtattcgagggattgtatgacgcctggccacacaggagctaa

SEQ ID NO: 29 - protein, 620 aa, Penicillium coprophilum
  SIGNAL 1..17
  PEPTIDE 18..620
MKLSSAITVVLANRAFAVVYKSQIDYNSAPPNLSTLTNASLFETWRPRAHILPPSGKIGDPCGQYTDPKTGLFHVGWLHEGIAGATTDDLATYRDLNPDGAPSIIAGGKNDPLAVFDGSVIPSGIDGMPTLLYTSVSYLPIHWSIPYTRGSETQSLAVSYDGGRNFTKLDQGPMIPSPPFAVNVTAFRDPYAFQSPALDIAVNSTRGTWYTAISGGVHDVGPCQFLYRQHDADFQYWEYLGQWWSEPVNTTWGNGDWAGGWGFNFEVGNVFSLNADGYSEDGEVFMTLGTESSEVPIVPKVSSIHDMVWVAGNITNDGSVTFNPTMAGVLDWGLSAYAAAGKILPASSQASRKSGAPDRFVSYTWLTGDLYELSKGFPSAQQNWTGTLLLPRELNVRTIRNVVDNKLSRESLASWRVAREGHGQIDLETMGISIARETYSALTSGSSSVESDQTLSEAGSKAFSISPSTKFFVLTANISFPTSARDSGIQSGFQILSSDLESTTIYYQFSNESIIVDRSNTSAASKTTDGISSANEAGRLRLFDVLQNGKKQIETLELTVIVDNGVLEIYANGRFALSTWARSWYANSTGINFFHNGIGETTFGDVKVFEGLYDAWPHRS

SEQ ID NO: 30 - protein, 603 aa, Penicillium coprophilum
VVYKSQIDYNSAPPNLSTLTNASLFETWRPRAHILPPSGKIGDPCGQYTDPKTGLFHVGWLHEGIAGATTDDLATYRDLNPDGAPSIIAGGKNDPLAVFDGSVIPSGIDGMPTLLYTSVSYLPIHWSIPYTRGSETQSLAVSYDGGRNFTKLDQGPMIPSPPFAVNVTAFRDPYAFQSPALDIAVNSTRGTWYTAISGGVHDVGPCQFLYRQHDADFQYWEYLGQWWSEPVNTTWGNGDWAGGWGFNFEVGNVFSLNADGYSEDGEVFMTLGTESSEVPIVPKVSSIHDMVWVAGNITNDGSVTFNPTMAGVLDWGLSAYAAAGKILPASSQASRKSGAPDRFVSYTWLTGDLYELSKGFPSAQQNWTGTLLLPRELNVRTIRNVVDNKLSRESLASWRVAREGHGQIDLETMGISIARETYSALTSGSSSVESDQTLSEAGSKAFSISPSTKFFVLTANISFPTSARDSGIQSGFQILSSDLESTTIYYQFSNESIIVDRSNTSAASKTTDGISSANEAGRLRLFDVLQNGKKQIETLELTVIVDNGVLEIYANGRFALSTWARSWYANSTGINFFHNGIGETTFGDVKVFEGLYDAWPHRS

SEQ ID NO: 31 - DNA, 1924 bp, Penicillium murcianum
  source 1..1924: mol_type = genomic DNA
  exon 1..1754
  intron 1755..1806
  exon 1807..1924
atgaagttgtccagtgcgattttcgtctttttggccgaccgagcagcatgtgctgtcttcagtcgtgaggtttcctcggttgattacaactcggcgcctccaaacctatcgacattagccaatgccactctatttaacacatggagaccgagagctcatatactccctccatctggaaagataggcgatccgtgcatgcaatacactgatcctaagacaggactcttccatgttggctggctccacgacggaattgcaggcgccaccacagatgatctcgctacgtacacggatttgatgcccgacggacttccttcaattgttgcaggaggaaaaaatgatccacttgctgtcttcgatggctcagtgatccccaatggcattgatggtctgccaacattgctatatacttcagtgtcttatcttccaattcactggtctattccctacacccggggaagcgaaacccagtccttggccgttacctatgatggtggtcgcaacttcaccaagctcagccaaggcccggtcatcccttcgccaccatttgccctcaatgtgacggcattccgcgacccatatgccttccaaaacccggcattggattcagccctcaacagtacccaaggaacttggtataccgccatctctggcggtgttcacaatatcggaccttgccagttcctctatcgccagcatgatgcaggcttccagtactgggaatatctcgggcaatggtggcaggagccggtcaacacaacctggggtaatggtgactgggctggaggatggggcttcaactttgaggttggcaacgtcttcagcttgaatgaagacgggtacagcgaggatggagaattattcatgacactcggcactgagggatcagaggcacctgtcactccaagagttacttccatccatcacatgttgtgggtaacaggaaacgtcaccaagggagattctgtcgctttcacgcccaccatggcaggtctccttgactggggcttgtcatcatatgcagctgcaggaaagatcctaccagcgacttctaaggcttctgctaagagcggcgcccccgatcgcttcatctcatacgtctggctcactggagatcaatttgagttggcaactggcttccctaccgctcagcaaaactggaccggaacccttcttctcccacgcgagttgaaggttgaaactcttcccaatgttgtagacaatgagcttgcacgcgagccactgtcttcctggcgcgtgtctcgcgaggattctggccaggttgatctccagactatgggaatctcaattgccagggagacttacaatgctctgacatctgggtcctcttctgtcgaggcaggtcggacattgtccgaagctggagacgtggcgtttgaaacctcaccatcaagcaagttcttcgttctgaccgcaaacatttctttcccgagctcagcccgtggctcagatcttcaatccgggttcaagattctttcgtcagaacttgagtcaacgtccatctattaccaattctcaaacgagtccgtcattatcgaccgcagcaacacaagtgctgcagcaaagactaccgatggaatcagcagcgacaacgaggcaggtcgcctacgtcttttcgatatcgggcagaatggagctgaaaatattgagaccctggagctcactatcgtggtggatagtggcgttctggaggtttatgccaatcgacgcttcgcgttgagtacctgggcacggtaagtacatctagtgtcgtcacataacacagctcactaacttgatattaaggtcctggtatacaaactcgaccgatattagctttttccacaacggtgtgggagaagtaacctttggaaatgtgacaatctttgaaggactgtacgatgcctggccccagagaggataa

SEQ ID NO: 32 - protein, 623 aa, Penicillium murcianum
  SIGNAL 1..17
  PEPTIDE 18..623
MKLSSAIFVFLADRAACAVFSREVSSVDYNSAPPNLSTLANATLFNTWRPRAHILPPSGKIGDPCMQYTDPKTGLFHVGWLHDGIAGATTDDLATYTDLMPDGLPSIVAGGKNDPLAVFDGSVIPNGIDGLPTLLYTSVSYLPIHWSIPYTRGSETQSLAVTYDGGRNFTKLSQGPVIPSPPFALNVTAFRDPYAFQNPALDSALNSTQGTWYTAISGGVHNIGPCQFLYRQHDAGFQYWEYLGQWWQEPVNTTWGNGDWAGGWGFNFEVGNVFSLNEDGYSEDGELFMTLGTEGSEAPVTPRVTSIHHMLWVTGNVTKGDSVAFTPTMAGLLDWGLSSYAAAGKILPATSKASAKSGAPDRFISYVWLTGDQFELATGFPTAQQNWTGTLLLPRELKVETLPNVVDNELAREPLSSWRVSREDSGQVDLQTMGISIARETYNALTSGSSSVEAGRTLSEAGDVAFETSPSSKFFVLTANISFPSSARGSDLQSGFKILSSELESTSIYYQFSNESVIIDRSNTSAAAKTTDGISSDNEAGRLRLFDIGQNGAENIETLELTIVVDSGVLEVYANRRFALSTWARSWYTNSTDISFFHNGVGEVTFGNVTIFEGLYDAWPQRG

SEQ ID NO: 33 - protein, 606 aa, Penicillium murcianum
AVFSREVSSVDYNSAPPNLSTLANATLFNTWRPRAHILPPSGKIGDPCMQYTDPKTGLFHVGWLHDGIAGATTDDLATYTDLMPDGLPSIVAGGKNDPLAVFDGSVIPNGIDGLPTLLYTSVSYLPIHWSIPYTRGSETQSLAVTYDGGRNFTKLSQGPVIPSPPFALNVTAFRDPYAFQNPALDSALNSTQGTWYTAISGGVHNIGPCQFLYRQHDAGFQYWEYLGQWWQEPVNTTWGNGDWAGGWGFNFEVGNVFSLNEDGYSEDGELFMTLGTEGSEAPVTPRVTSIHHMLWVTGNVTKGDSVAFTPTMAGLLDWGLSSYAAAGKILPATSKASAKSGAPDRFISYVWLTGDQFELATGFPTAQQNWTGTLLLPRELKVETLPNVVDNELAREPLSSWRVSREDSGQVDLQTMGISIARETYNALTSGSSSVEAGRTLSEAGDVAFETSPSSKFFVLTANISFPSSARGSDLQSGFKILSSELESTSIYYQFSNESVIIDRSNTSAAAKTTDGISSDNEAGRLRLFDIGQNGAENIETLELTIVVDSGVLEVYANRRFALSTWARSWYTNSTDISFFHNGVGEVTFGNVTIFEGLYDAWPQRG

SEQ ID NO: 34 - DNA, 1925 bp, Penicillium venetum
  source 1..1925: mol_type = genomic DNA
atgaagctctcaagtgtaatgaccgtgggtttggcaaaccgggtactagctgccaaccctcatgcctccccaattgactacaactcggctccaccaaacctgtctaccttggcgaatgaaaccctgttcgacacctggagacccaaagcgcatctcctccctccatccgggaagatcggcgacccatgcgggcattacaccgatcctaagactggcctcttccatgtgggctggctgcacgacggaattgcaggagctacaaccgatgatctcgctacctacaaggatcttaatcccgatggagctccggtaattatcgccggaggaaagaacgaccctcttgctgtctttgacggctccgtcattcccagcggtatcaatggcttgccaaccctgctgtatacctctgtctcgtatctcccaattcactggtctatcccctacacccggggaagcgagacacaatccctggccgtctcccatgacggtggacgcaacttcactaagctcaaccaaggccccgtgatccccacacccccctttgctgtcaatgtcaccgctttccgtgacccctttgctttccaaagctccattctggacaaatctgtcaacagtactaaaggaacctggtatgtcgccatctctggcggtgtccacgatgtcggacctactcagttcctctaccgtcagcacgacgcagacttccagtactgggaatatctcggccagtggtggaatgagcccgtcaacacaacttggggcaagggtgactgggccggcggctggggcttcaactttgaggttggcaatgtcttcggtctgaatgcagaggggtacagcgaggacggtgaaatcttcattaccctcggtacggagggttccggactccccatcgttccccaggtctcttccatccacgatatgctgtgggcgactggaaatgtcactaatgacgactccgtcaccttcaagccgaccatggccggtgtgcttgactggggtctgtcggcctatgctgctgcaggcaaggtcttgccagccagctcccaagcatccgaaaagagcggtgcccccgatcgtttcatttcctacgtctggctcaccggagatctattcgagctagcgaagggattccccactgctcaacaaaactggaccggcactctcttgctgccgcgtgagctgaatatccgcaccatccccaacgtggttgacaatgagctttcccgcgagtccttgacgtcgtggcgtgtggcccgcgaaggatctggtgaggtcgatctcgagacaatgggaatctcaattgccagggagacttacagcgctctcacatccggcccttcttttgtcgagtctggtaagaccttgtccaaggctggttccgtgcctttcaacacctcgccctcgaccaagttcttcgctctgacagccaacatctctttcccggcctctgctcgtgactccggtattcaagctggtttccaggtcttgtcctctggtcttgagtctaccactatctactaccaattctccaacgagtccatcatcatcgaccgcagcaacacaagtgccgcgtcgaagacaactaatggaattctcagtgacaacgaggcgggccgtcttcgtctctttgatgtcctggaaaatggaaaggaaaaagttgagaccttggagctcaccgtcgtggtggataatggtgttctggaagtctatgccaatggacgctttgctctgggtacttgggctcggtaggtttttcttttcttttttttctctgtgagattgatcaacaactaataacatgcaggtcttggtatgccaactcgactgagatcaaattcttccacaacggtgttggagaggcgacatttgggaatgtgacaatcttcgagggactgtatgacgcctggccacaaaggaagtaa

SEQ ID NO: 35 - protein, 621 aa, Penicillium venetum
  SIGNAL 1..17
  PEPTIDE 18..621
MKLSSVMTVGLANRVLAANPHASPIDYNSAPPNLSTLANETLFDTWRPKAHLLPPSGKIGDPCGHYTDPKTGLFHVGWLHDGIAGATTDDLATYKDLNPDGAPVIIAGGKNDPLAVFDGSVIPSGINGLPTLLYTSVSYLPIHWSIPYTRGSETQSLAVSHDGGRNFTKLNQGPVIPTPPFAVNVTAFRDPFAFQSSILDKSVNSTKGTWYVAISGGVHDVGPTQFLYRQHDADFQYWEYLGQWWNEPVNTTWGKGDWAGGWGFNFEVGNVFGLNAEGYSEDGEIFITLGTEGSGLPIVPQVSSIHDMLWATGNVTNDDSVTFKPTMAGVLDWGLSAYAAAGKVLPASSQASEKSGAPDRFISYVWLTGDLFELAKGFPTAQQNWTGTLLLPRELNIRTIPNVVDNELSRESLTSWRVAREGSGEVDLETMGISIARETYSALTSGPSFVESGKTLSKAGSVPFNTSPSTKFFALTANISFPASARDSGIQAGFQVLSSGLESTTIYYQFSNESIIIDRSNTSAASKTTNGILSDNEAGRLRLFDVLENGKEKVETLELTVVVDNGVLEVYANGRFALGTWARSWYANSTEIKFFHNGVGEATFGNVTIFEGLYDAWPQRK

SEQ ID NO: 36 - protein, 604 aa, Penicillium venetum
ANPHASPIDYNSAPPNLSTLANETLFDTWRPKAHLLPPSGKIGDPCGHYTDPKTGLFHVGWLHDGIAGATTDDLATYKDLNPDGAPVIIAGGKNDPLAVFDGSVIPSGINGLPTLLYTSVSYLPIHWSIPYTRGSETQSLAVSHDGGRNFTKLNQGPVIPTPPFAVNVTAFRDPFAFQSSILDKSVNSTKGTWYVAISGGVHDVGPTQFLYRQHDADFQYWEYLGQWWNEPVNTTWGKGDWAGGWGFNFEVGNVFGLNAEGYSEDGEIFITLGTEGSGLPIVPQVSSIHDMLWATGNVTNDDSVTFKPTMAGVLDWGLSAYAAAGKVLPASSQASEKSGAPDRFISYVWLTGDLFELAKGFPTAQQNWTGTLLLPRELNIRTIPNVVDNELSRESLTSWRVAREGSGEVDLETMGISIARETYSALTSGPSFVESGKTLSKAGSVPFNTSPSTKFFALTANISFPASARDSGIQAGFQVLSSGLESTTIYYQFSNESIIIDRSNTSAASKTTNGILSDNEAGRLRLFDVLENGKEKVETLELTVVVDNGVLEVYANGRFALGTWARSWYANSTEIKFFHNGVGEATFGNVTIFEGLYDAWPQRK

SEQ ID NO: 37 - DNA, 1914 bp, Curvularia spicifera
  source 1..1914: mol_type = genomic DNA
  exon 1..1914
atgaagctctcgtcacccgttttggtggtgccattggcaacactagcgacaagtcggatcttgcctcgtcaaaacgccatcgactacaacgctgctccgccaaacctttctactctagccaacaactctctcttcgacacatggcggcccaaagcacacgtgctccctgcttttggccaaattggagatccttgcatgctctacaccgatccggagacaggcctttttcacgtaggataccttcatctaggcgcatcaggcgctaccaccaaggatttggttacctacaaggacttgaatcccgactcagctcctttcattcgtgcaggtggaatcaacgatcccgtcgcagtcttcgacggcagcgtcattgaaaagggtgtcaacggcacacctacccttctctacaccagcgttagctatctacccatccactggaccatcagatatccaaagggtgctgaaacgcaatctctagcctatgccagagatggaggaagaaattttacaaaagctagatttggtcctgtaatccctagcccaccgtttgctgtcaatgtgaccgctttccgagacccgtacgtgtttcaaagcccgcagattgatgccttgcttggaaaaaagaaagacacatggtacactgtcatctcaggcggcgtccatggtgtcggccccagcatgttcctgtacgcgcagtacgaaaaagatgaagacgagtttcagacctgggagtacctaggcgagtggtggaacgaacctgcgaattcgacctggacagagcatggctgggccggccgctggggcttcaattttgaagttgccaacgtgttttccctggacgaacgcggcgccaattctgagggcgaaacgttcatgactctaggcgcagaatggagccatgcgcctattattccccaggtctctgaagagagagacatgctatgggtcgctggcaagaaatcccttgtcgacggtaagttgaagttcgagccgaccatggcaggtagaatggatgcaggccgctccgcctatgctgccgcgggcaaagttgtacctgcagattcgcagaccagctgggcgagtggtgctcccgaccgcgccatctcatatctctggcttacaggcgacttatacggaagctccgatttccctattactgcacagcaaaactggacaggttcgcttctgcttccccgtgaactcagccgctgccacctcgacgtcatcgacaacacgttctcccgcgaaaaaggctcctggagaatcgagcaagaccagcagtccggcagcagcaaaggcgctacggtccgcctcaccaccctatgccagaaaatcgcccgagaagtcacctctgcattcaaagcccaagctaccaacatcatcaccctccccggcggcacccaagcaagcggaaccaagcaaaaactgcctcaaacacccaaatcaaaccattacatgctcaccgcctccatcactttcccctcgcgctctgaaggcttaaaatcaggcttctccattctctctggtgcccacgagtccactcgcgtactctacgatatatcccgcgagcacctcgtcgtcgagcgcaccaactcctccgctgcagcaagcactacatctgggctccccacgtacgacgaatcaggtctgttccgcctattcgatgttcccagcccgagcgccaacggaacgcaagtcgaaacgctcaatctgacaattgtggttgacgccggtgttatcgaagtacatgctaatgaccgttttgcgctgagtacgtgggtcaggccgtggtatgctgatagtagggatatcgcgttctttgtagagggcggggatgtgcagctgggtaatgtgacggtgtatgaggggctggtggatgcgtggccttccaggaacaagaagaaggtggcgtaa

SEQ ID NO: 38 - protein, 637 aa, Curvularia spicifera
  SIGNAL 1..23
  PEPTIDE 24..637
MKLSSPVLVVPLATLATSRILPRQNAIDYNAAPPNLSTLANNSLFDTWRPKAHVLPAFGQIGDPCMLYTDPETGLFHVGYLHLGASGATTKDLVTYKDLNPDSAPFIRAGGINDPVAVFDGSVIEKGVNGTPTLLYTSVSYLPIHWTIRYPKGAETQSLAYARDGGRNFTKARFGPVIPSPPFAVNVTAFRDPYVFQSPQIDALLGKKKDTWYTVISGGVHGVGPSMFLYAQYEKDEDEFQTWEYLGEWWNEPANSTWTEHGWAGRWGFNFEVANVFSLDERGANSEGETFMTLGAEWSHAPIIPQVSEERDMLWVAGKKSLVDGKLKFEPTMAGRMDAGRSAYAAAGKVVPADSQTSWASGAPDRAISYLWLTGDLYGSSDFPITAQQNWTGSLLLPRELSRCHLDVIDNTFSREKGSWRIEQDQQSGSSKGATVRLTTLCQKIAREVTSAFKAQATNIITLPGGTQASGTKQKLPQTPKSNHYMLTASITFPSRSEGLKSGFSILSGAHESTRVLYDISREHLVVERTNSSAAASTTSGLPTYDESGLFRLFDVPSPSANGTQVETLNLTIVVDAGVIEVHANDRFALSTWVRPWYADSRDIAFFVEGGDVQLGNVTVYEGLVDAWPSRNKKKVA

SEQ ID NO: 39 - protein, 614 aa, Curvularia spicifera
QNAIDYNAAPPNLSTLANNSLFDTWRPKAHVLPAFGQIGDPCMLYTDPETGLFHVGYLHLGASGATTKDLVTYKDLNPDSAPFIRAGGINDPVAVFDGSVIEKGVNGTPTLLYTSVSYLPIHWTIRYPKGAETQSLAYARDGGRNFTKARFGPVIPSPPFAVNVTAFRDPYVFQSPQIDALLGKKKDTWYTVISGGVHGVGPSMFLYAQYEKDEDEFQTWEYLGEWWNEPANSTWTEHGWAGRWGFNFEVANVFSLDERGANSEGETFMTLGAEWSHAPIIPQVSEERDMLWVAGKKSLVDGKLKFEPTMAGRMDAGRSAYAAAGKVVPADSQTSWASGAPDRAISYLWLTGDLYGSSDFPITAQQNWTGSLLLPRELSRCHLDVIDNTFSREKGSWRIEQDQQSGSSKGATVRLTTLCQKIAREVTSAFKAQATNIITLPGGTQASGTKQKLPQTPKSNHYMLTASITFPSRSEGLKSGFSILSGAHESTRVLYDISREHLVVERTNSSAAASTTSGLPTYDESGLFRLFDVPSPSANGTQVETLNLTIVVDAGVIEVHANDRFALSTWVRPWYADSRDIAFFVEGGDVQLGNVTVYEGLVDAWPSRNKKKVA

SEQ ID NO: 40 - DNA, 1881 bp, Alternaria sp.
  source 1..1881: mol_type = genomic DNA
atgaagcgctcatcaaccgttctggcgtctcttgcagcactgacgacaagccatattatccctcgtcaagaatccatcgactacaatgcagctccgccaaatctgtccaccttagccaattcgactctcttcgatacatggcggccaaaggcacacgttcttccagcgtttggacaaatcggagatccatgtatgcactacaccgatcccgaaactggtctcttccacgtaggatacctacatttgggagcatcaggtgctactacagacaacctagtcacttacagggacgtgaatcccgattcggagccgttcattcgtgcaggcggcatcaatgaccccgtcgctgtctttgacggcagtgttattgagagaggagttaatggcacaccgacgttgctatacaccagcgtcagctttctacccatacaatggacaattcggtacacccaaggaagcgagacgcaatcgctcgcgttcgctagagacggtggaagcaacttcacaaaagcagaatttggtccggttatccccagcccaccattcgctgtcaacgtcacgggattccgcgatccattcgtgtttcagaactcgcaggttgatggcttgttgggtaagcggaacggaacatggtatacagtcatttctggcggcgtacatggcaaaggcccaagtctcttcttgtacgcccaatatgatgatgaagatgcggatttgttccagacgtgggagtatttgggtgaatggtggcacgaagcagcaaactcgacatggtcacagacgggctgggctgggcgctggggcttcaactttgaagtcgccaactacttcgccctggatgagcaaggtgctaatgaggaaggcgagatcttcgccacaatcggcgcggaatggagctacgaacccattgtacctcaagtttcggacaacagggagatgctttgggtcgctggaaagcaagagcttgtcgatggtcaactgaagttcgagcctacaatgtccggacgactcgatgctgggcgctcagcttacgcagcggcaggtaaagtcttatatgcagactcgcaagctagcaaagcaagtggcgcgcctgatcgcttcatcacatatctctggttgactggcgacttctatggcaccttgactgacgaattcccgactgcccagcaaaactggacaggatcactattactcccgcgtgagcttagcgtcggccgcttgaatgttgtcgacaaccagctctcccgagagaagggttcttggcgtgtagatcaggaacacgacaacggtaccgttaccctcgccactctacaccaaaagatcgctcgtgaaccctacgccgcattccaagacaacgccaccaatgtcattacgcaaacgggtggatccatggcgagcgtgatgaaattcgatgaaagcccaaagtcaaagcactggatgatgactgcatcgataaccttcccctcgcgctctgactccctgcgagctggcttcaccatcctttctggttcccaggaatctaccaatatctactaccagttcggtaatgagagtcttatcatcgatcgttcaaactcttctgccgcggcacagactacggatggtttcctcaccgagattgagacgggcaaattccgcttgttcgatgttgcgggtggatatggaaatgagacaaaggttgagactttggatctgacgattgtggtcgacggtggtgttgttgaggtgcatgcgaacgatcgatttgcgatcagtacttgggtgaggagctggtacgcggatagcagagacattagtttctatgtcgagggtggcgatgctaccttcagcaatgtaacgatttacgagggtttagttgacgcatggccggagcgaaattaa

SEQ ID NO: 41 - protein, 626 aa, Alternaria sp.
  SIGNAL 1..22
  PEPTIDE 23..626
MKRSSTVLASLAALTTSHIIPRQESIDYNAAPPNLSTLANSTLFDTWRPKAHVLPAFGQIGDPCMHYTDPETGLFHVGYLHLGASGATTDNLVTYRDVNPDSEPFIRAGGINDPVAVFDGSVIERGVNGTPTLLYTSVSFLPIQWTIRYTQGSETQSLAFARDGGSNFTKAEFGPVIPSPPFAVNVTGFRDPFVFQNSQVDGLLGKRNGTWYTVISGGVHGKGPSLFLYAQYDDEDADLFQTWEYLGEWWHEAANSTWSQTGWAGRWGFNFEVANYFALDEQGANEEGEIFATIGAEWSYEPIVPQVSDNREMLWVAGKQELVDGQLKFEPTMSGRLDAGRSAYAAAGKVLYADSQASKASGAPDRFITYLWLTGDFYGTLTDEFPTAQQNWTGSLLLPRELSVGRLNVVDNQLSREKGSWRVDQEHDNGTVTLATLHQKIAREPYAAFQDNATNVITQTGGSMASVMKFDESPKSKHWMMTASITFPSRSDSLRAGFTILSGSQESTNIYYQFGNESLIIDRSNSSAAAQTTDGFLTEIETGKFRLFDVAGGYGNETKVETLDLTIVVDGGVVEVHANDRFAISTWVRSWYADSRDISFYVEGGDATFSNVTIYEGLVDAWPERN

SEQ ID NO: 42 - protein, 604 aa, Alternaria sp.
QESIDYNAAPPNLSTLANSTLFDTWRPKAHVLPAFGQIGDPCMHYTDPETGLFHVGYLHLGASGATTDNLVTYRDVNPDSEPFIRAGGINDPVAVFDGSVIERGVNGTPTLLYTSVSFLPIQWTIRYTQGSETQSLAFARDGGSNFTKAEFGPVIPSPPFAVNVTGFRDPFVFQNSQVDGLLGKRNGTWYTVISGGVHGKGPSLFLYAQYDDEDADLFQTWEYLGEWWHEAANSTWSQTGWAGRWGFNFEVANYFALDEQGANEEGEIFATIGAEWSYEPIVPQVSDNREMLWVAGKQELVDGQLKFEPTMSGRLDAGRSAYAAAGKVLYADSQASKASGAPDRFITYLWLTGDFYGTLTDEFPTAQQNWTGSLLLPRELSVGRLNVVDNQLSREKGSWRVDQEHDNGTVTLATLHQKIAREPYAAFQDNATNVITQTGGSMASVMKFDESPKSKHWMMTASITFPSRSDSLRAGFTILSGSQESTNIYYQFGNESLIIDRSNSSAAAQTTDGFLTEIETGKFRLFDVAGGYGNETKVETLDLTIVVDGGVVEVHANDRFAISTWVRSWYADSRDISFYVEGGDATFSNVTIYEGLVDAWPERN

SEQ ID NO: 43 - DNA, 1904 bp, Fusarium temperatum
  source 1..1904: mol_type = genomic DNA
atgaagttctcacttttcttgacggctggctctcttgcctgggccaagccatcaactccaaagcttgactataaccgcgcgcccccaaatctcagcactctggccaatctgaccatctacaacacatggcgtcctcgagctcacgtactccctcctactggacagattggagatccttgcatgcactacactgatcccaaaactggactcttccacgttggatatctccatgagggtgctgcgggtgctacgacggatgatctcgtgacatatcgagatctcaaccctggaggcgctcctttcattcgggcaggaggtctaaatgatcctgtggcagtctttgacggatccgttatacccaagggcatcaatggcacgcctacactcttctacacctctgtatcttacctccccatccattggaccatcaactacactcgcggaagtgagacgcagtccctcgccgtgtcaaaggacggaggccgtaacttcacgaagctacgacagggccccgcgattccagctcctccctttgccctcaacgtgacggccttccgtgacccctttgttttccagaacgaagatcttgacagccttctcgaaagcgagtcaggaacatggtacagcgtcatttctggtggtgtccatcaaaagggccctagcatgtttctctatcgtcagcacgaccccgagtttcagtattgggagtatctcggcgagtggtggcatgaaaaggccaacactacatggggtgatggtctctgggcaggacgttacggtttcaactttgaagtggccaatgtcttcagtattgatgagaaggggtataactctgacggcgaaaccttcatcacatttggagccgagtggcaggaaaagcctattgttcctcaggtgtcgtctttccgcgacatgctgtgggctgcaggaaacatatccctcgacaacggaaagccaaagtttaccccttccatggttggcaagcttgactggggtcgatccgcttacgccgcggctggcaaatatctcccttcagactcaaaggcatcgtccaagagcggtgcaccagatcgattcatcagctacctttggcttactggagattactacggtgatctcaagtatttccctactccccagcagaattggactgggtctcttctcctccccagagagctcaccgtcggtaagatcagtaatgtcgtcgataatgagctttcacgtgaagaaggatcctggcgtattgagagaaatgagtcgggtgttttggaacttgctactctgaagcaggttattgctcgggagcctatggctgcattcaccaagaagacctcttttgttgaaccgggccgaaacatcagcaaggctggatcaactacattcgaccataacccggagtccaagttttacgtcctcaagagctctatctcattccccaaatctgctcgtggctcggacctcaaggctggtttccagattttggcttcggaaaaggagtccaccactatttactaccagttctccaacgaatccatcataatcgatcgcagcaacactagtgctgcagctcttacaactagcgacattgacgctcgccctgaagctggtcgactccgtctttttgacgtctttgttgatgatgaggaaaagattgagacacttgatcttactattgtcgttgataatgccatcgttgaggtccatgccaacgatagatttggagttgccacctgggctcggtaagttgggatatcccctgatgctgtgatacttgctgactttttagttcttggtatgacgattcgactgacattcgcttcttccataagggaagtggtgaggtgtccttcagcaatgtgacgatttatgagggtcttgcggatgcttggcctgagagaaagtaa

SEQ ID NO: 44 - protein, 618 aa, Fusarium temperatum
  SIGNAL 1..15
  PEPTIDE 16..618
MKFSLFLTAGSLAWAKPSTPKLDYNRAPPNLSTLANLTIYNTWRPRAHVLPPTGQIGDPCMHYTDPKTGLFHVGYLHEGAAGATTDDLVTYRDLNPGGAPFIRAGGLNDPVAVFDGSVIPKGINGTPTLFYTSVSYLPIHWTINYTRGSETQSLAVSKDGGRNFTKLRQGPAIPAPPFALNVTAFRDPFVFQNEDLDSLLESESGTWYSVISGGVHQKGPSMFLYRQHDPEFQYWEYLGEWWHEKANTTWGDGLWAGRYGFNFEVANVFSIDEKGYNSDGETFITFGAEWQEKPIVPQVSSFRDMLWAAGNISLDNGKPKFTPSMVGKLDWGRSAYAAAGKYLPSDSKASSKSGAPDRFISYLWLTGDYYGDLKYFPTPQQNWTGSLLLPRELTVGKISNVVDNELSREEGSWRIERNESGVLELATLKQVIAREPMAAFTKKTSFVEPGRNISKAGSTTFDHNPESKFYVLKSSISFPKSARGSDLKAGFQILASEKESTTIYYQFSNESIIIDRSNTSAAALTTSDIDARPEAGRLRLFDVFVDDEEKIETLDLTIVVDNAIVEVHANDRFGVATWARSWYDDSTDIRFFHKGSGEVSFSNVTIYEGLADAWPERK

SEQ ID NO: 45 - protein, 603 aa, Fusarium temperatum
KPSTPKLDYNRAPPNLSTLANLTIYNTWRPRAHVLPPTGQIGDPCMHYTDPKTGLFHVGYLHEGAAGATTDDLVTYRDLNPGGAPFIRAGGLNDPVAVFDGSVIPKGINGTPTLFYTSVSYLPIHWTINYTRGSETQSLAVSKDGGRNFTKLRQGPAIPAPPFALNVTAFRDPFVFQNEDLDSLLESESGTWYSVISGGVHQKGPSMFLYRQHDPEFQYWEYLGEWWHEKANTTWGDGLWAGRYGFNFEVANVFSIDEKGYNSDGETFITFGAEWQEKPIVPQVSSFRDMLWAAGNISLDNGKPKFTPSMVGKLDWGRSAYAAAGKYLPSDSKASSKSGAPDRFISYLWLTGDYYGDLKYFPTPQQNWTGSLLLPRELTVGKISNVVDNELSREEGSWRIERNESGVLELATLKQVIAREPMAAFTKKTSFVEPGRNISKAGSTTFDHNPESKFYVLKSSISFPKSARGSDLKAGFQILASEKESTTIYYQFSNESIIIDRSNTSAAALTTSDIDARPEAGRLRLFDVFVDDEEKIETLDLTIVVDNAIVEVHANDRFGVATWARSWYDDSTDIRFFHKGSGEVSFSNVTIYEGLADAWPERK

SEQ ID NO: 46 - DNA, 1962 bp, Aspergillus japonicus
  source 1..1962: mol_type = genomic DNA
atgaagctccccaccaccaccctggcgctcgccaccggcgcagcagcagcagcctcataccacgtggacaccacggccccgccgcccaccaacctgagcaccctccccaacaacaccctcttccacctgtggcggccgcgcgcgcacatcctgcccgccgagggccagatcggcgacccctgcgcgcactacaccgacccctccaccggcctcttccacgtcgggttcctgcacaacggggacggtatcgcgggtgccaccacggccaacctggccacctacaccgacacctccggcaacgggagcttcctgatccagccgggcgggaagaacgaccccgtcgccgtgttcgacggcgccgtcatccccgccggcgtcaacaacacccccaccttactctacacctccgtctccttcctgcccatccactggtccatcccctacacccgcggcagcgagacgcagtcgctggccgtcgcgcgcgacggcggccgccgcttcgagaagctcgaccagggccccgtcatcgccgaccatcccttcgccgtcgatgtcaccgcgttccgtgatccgtttgtcttccgcagtgccaggttggatgtgctgttgtcgttggatcaagaggtggcagggaatgagacggccgtgcagcaggctgtcgatggctggacggagaagaacgccccctggtatgtcgctgtctctggtggggtgcacggcgtcgggcccgcgcagttcctctaccgccagaacggcgggaatggttccgagttccagtactgggagtaccttggggagtggtggcaggaggcgaccaactccagctggggcgaggagggcacctgggctgggcgctgggggttcaatttcgagacggggaatgtgctcttcctcacggaggagggccatgaccccaagactggcgaggtgttcgtcaccctcggcactgaggggtctggcctgcccatcgtgccgcaggtctctagtatccatgatatgctgtgggcggcgggtgaggtcggggttggcagtgagcaggggggtgccacggtcgagttctccccctccatggccgggtttctggactgggggttcagtgcctacgctgcggcgggcaaggtgctgccggccagctcggcggtgtcgaagactagcggcgtggatgtggatcggtatatctcgttcgtctggttgacgggcgatcagtacgagcaggcggacgggtttccgacagctcagcaggggtggacggggtcgctgctgctgccgcgcgagctgaaggtgcagacggtggagaacgtcgttgacaacgagctggtgcgcgaggagggcgtgtcctgggttgtgggggagtcgaacaacaagacggccacgctgcgcacgctggggatcacgatcgccagggagaccaaggcggccctgctggccaacggctcggtgaccacggaggaggcccgcacgttgcagacggcggccgtcgtgccgttcgcgcagtcgccgggctccaagttcttcgtgctgacagcgcagctggagttccccgcgagcgcgcgctcgtccaccctccagtccgggttcgagatcctggcgtccgagctggagcgcacgaccgtctactaccagttcagcaatgagtcgctggtcgtcgaccgcagccagaccagtgcggcggcgcccacgaaccccgggctggatagctttactgagtccggcaagttgcggttgttcgatgtggtcgagaacggccagaagacggtcgagacgttggatctcactgtcgtggtggataactcggttgttgaggtgtatgccaacgggcgctttgcgttgagcacctgggcgagatcgtggtacgacaactccacccagatccgcttcttccacaacggcgagggcgaggtgcagttcaagaatgtctccgtgtcggaggggctctataacgcctggccggagagagattaa

SEQ ID NO: 47 - protein, 653 aa, Aspergillus japonicus
  SIGNAL 1..19
  PEPTIDE 20..653
MKLPTTTLALATGAAAAASYHVDTTAPPPTNLSTLPNNTLFHLWRPRAHILPAEGQIGDPCAHYTDPSTGLFHVGFLHNGDGIAGATTANLATYTDTSGNGSFLIQPGGKNDPVAVFDGAVIPAGVNNTPTLLYTSVSFLPIHWSIPYTRGSETQSLAVARDGGRRFEKLDQGPVIADHPFAVDVTAFRDPFVFRSARLDVLLSLDQEVAGNETAVQQAVDGWTEKNAPWYVAVSGGVHGVGPAQFLYRQNGGNGSEFQYWEYLGEWWQEATNSSWGEEGTWAGRWGFNFETGNVLFLTEEGHDPKTGEVFVTLGTEGSGLPIVPQVSSIHDMLWAAGEVGVGSEQGGATVEFSPSMAGFLDWGFSAYAAAGKVLPASSAVSKTSGVDVDRYISFVWLTGDQYEQADGFPTAQQGWTGSLLLPRELKVQTVENVVDNELVREEGVSWVVGESNNKTATLRTLGITIARETKAALLANGSVTTEEARTLQTAAVVPFAQSPGSKFFVLTAQLEFPASARSSTLQSGFEILASELERTTVYYQFSNESLVVDRSQTSAAAPTNPGLDSFTESGKLRLFDVVENGQKTVETLDLTVVVDNSVVEVYANGRFALSTWARSWYDNSTQIRFFHNGEGEVQFKNVSVSEGLYNAWPERD

SEQ ID NO: 48 - protein, 634 aa, Aspergillus japonicus
YHVDTTAPPPTNLSTLPNNTLFHLWRPRAHILPAEGQIGDPCAHYTDPSTGLFHVGFLHNGDGIAGATTANLATYTDTSGNGSFLIQPGGKNDPVAVFDGAVIPAGVNNTPTLLYTSVSFLPIHWSIPYTRGSETQSLAVARDGGRRFEKLDQGPVIADHPFAVDVTAFRDPFVFRSARLDVLLSLDQEVAGNETAVQQAVDGWTEKNAPWYVAVSGGVHGVGPAQFLYRQNGGNGSEFQYWEYLGEWWQEATNSSWGEEGTWAGRWGFNFETGNVLFLTEEGHDPKTGEVFVTLGTEGSGLPIVPQVSSIHDMLWAAGEVGVGSEQGGATVEFSPSMAGFLDWGFSAYAAAGKVLPASSAVSKTSGVDVDRYISFVWLTGDQYEQADGFPTAQQGWTGSLLLPRELKVQTVENVVDNELVREEGVSWVVGESNNKTATLRTLGITIARETKAALLANGSVTTEEARTLQTAAVVPFAQSPGSKFFVLTAQLEFPASARSSTLQSGFEILASELERTTVYYQFSNESLVVDRSQTSAAAPTNPGLDSFTESGKLRLFDVVENGQKTVETLDLTVVVDNSVVEVYANGRFALSTWARSWYDNSTQIRFFHNGEGEVQFKNVSVSEGLYNAWPERD

SEQ ID NO: 49 - DNA, 1449 bp, Bacillus amyloliquefaciens
  source 1..1449: mol_type = genomic DNA
gtaaatggcacgctgatgcagtattttgaatggtatacgccgaacgacggccagcattggaaacgattgcagaatgatgcggaacatttatcggatatcggaatcactgccgtctggattcctcccgcatacaaaggattgagccaatccgataacggatacggaccttatgatttgtatgatttaggagaattccagcaaaaagggacggtcagaacgaaatacggcacaaaatcagagcttcaagatgcgatcggctcactgcattcccggaacgtccaagtatacggagatgtggttttgaatcataaggctggtgctgatgcaacagaagatgtaactgccgtcgaagtcaatccggccaatagaaatcaggaaacttcggaggaatatcaaatcaaagcgtggacggattttcgttttccgggccgtggaaacacgtacagtgattttaaatggcattggtatcatttcgacggagcggactgggatgaatcccggaagatcagccgcatctttaagtttcgtggggaaggaaaagcgtgggattgggaagtatcaagtgaaaacggcaactatgactatttaatgtatgctgatgttgactacgaccaccctgatgtcgtggcagagacaaaaaaatggggtatctggtatgcgaatgaactgtcattagacggcttccgtattgatgccgccaaacatattaaattttcatttctgcgtgattgggttcaggcggtcagacaggcgacgggaaaagaaatgtttacggttgcggagtattggcagaataatgccgggaaactcgaaaactacttgaataaaacaagctttaatcaatccgtgtttgatgttccgcttcatttcaatttacaggcggcttcctcacaaggaggcggatatgatatgaggcgtttgctggacggtaccgttgtgtccaggcatccggaaaaggcggttacatttgttgaaaatcatgacacacagccgggacagtcattggaatcgacagtccaaacttggtttaaaccgcttgcatacgcctttattttgacaagagaatccggttatcctcaggtgttctatggggatatgtacgggacaaaagggacatcgccaaaggaaattccctcactgaaagataatatagagccgattttaaaagcgcgtaaggagtacgcatacgggccccagcacgattatattgaccacccggatgtgatcggatggacgagggaaggtgacagctccgccgccaaatcaggtttggccgctttaatcacggacggacccggcggatcaaagcggatgtatgccggcctgaaaaatgccggcgagacatggtatgacataacgggcaaccgttcagatactgtaaaaatcggatctgacggctggggagagtttcatgtaaacgatgggtccgtctccatttatgttcagaaa

SEQ ID NO: 50 - protein, 483 aa, Bacillus amyloliquefaciens
VNGTLMQYFEWYTPNDGQHWKRLQNDAEHLSDIGITAVWIPPAYKGLSQSDNGYGPYDLYDLGEFQQKGTVRTKYGTKSELQDAIGSLHSRNVQVYGDVVLNHKAGADATEDVTAVEVNPANRNQETSEEYQIKAWTDFRFPGRGNTYSDFKWHWYHFDGADWDESRKISRIFKFRGEGKAWDWEVSSENGNYDYLMYADVDYDHPDVVAETKKWGIWYANELSLDGFRIDAAKHIKFSFLRDWVQAVRQATGKEMFTVAEYWQNNAGKLENYLNKTSFNQSVFDVPLHFNLQAASSQGGGYDMRRLLDGTVVSRHPEKAVTFVENHDTQPGQSLESTVQTWFKPLAYAFILTRESGYPQVFYGDMYGTKGTSPKEIPSLKDNIEPILKARKEYAYGPQHDYIDHPDVIGWTREGDSSAAKSGLAALITDGPGGSKRMYAGLKNAGETWYDITGNRSDTVKIGSDGWGEFHVNDGSVSIYVQK

SEQ ID NO: 51 - protein, 29 aa, Bacillus licheniformis
MKQQKRLYARLLTLLFALIFLLPHSAAAA

SEQ ID NO: 52 - protein, 6 aa, synthetic construct
HHHHHH

(19) United States
(12) Patent Application Publication
(10) Pub. No.: US 2025/0161187 A1
(43) Pub. Date: May 22, 2025

(54) ORAL CARE COMPOSITION COMPRISING ENZYMES

(71) Applicant: Novozymes A/S, Bagsvaerd (DK)

(72) Inventors: Manish Kumar TIWARI, Charlottenlund (DK); Lorena González PALMÉN, Åkarp (SE); Dorotea Raventos SEGURA, Rungsted (DK); Marc MORANT, Frederiksberg C (DK); Anders Gunnar SANDSTRÖM, Södra Sandby (SE)

(73) Assignee: Novozymes A/S, Bagsvaerd (DK)

(21) Appl. No.: 18/839,676

(22) PCT Filed: Feb. 22, 2023
(86) PCT No.: PCT/EP2023/054360

§ 371 Date: Aug. 19, 2024

(30) Foreign Application Priority Data
Feb. 22, 2022 (DK) PA202200145

(51) Int. Cl. A61K 8/66; A61K 8/22; A61K 8/368; A61K 8/44; A61K 38/47; A61Q 11/00

(52) U.S. Cl. CPC A61K 8/66; A61K 8/22; A61K 8/368; A61K 8/44; A61K 38/47; A61Q 11/00; C12Y 302/01001; C12Y 302/01003; C12Y 302/01021; C12Y 302/01026; A61K 2800/92

(57) ABSTRACT

The present invention relates to an oral care composition comprising an invertase, a beta-glucosidase, and a glucoamylase, use of said composition as medicaments, use of said composition in treatment of oral disease, methods of treatment comprising administering said composition to a human subject, methods of preventing or removing oral biofilm comprising contacting an oral biofilm with said composition, methods for reducing the risk of oral biofilm formation, and kits of parts comprising said composition.

### REFERENCE TO SEQUENCE LISTING

This application contains a Sequence Listing in computer readable form. The computer readable form is incorporated herein by reference.

### FIELD OF THE INVENTION

The present invention relates to an oral care composition comprising an invertase, a beta-glucosidase, and a glucoamylase, use of said composition as medicaments, use of said composition in treatment of oral disease, methods of treatment comprising administering said composition to a human subject, methods of preventing or removing oral biofilm comprising contacting an oral biofilm with said composition, methods for reducing the risk of oral biofilm formation, and kits of parts comprising said composition.

### BACKGROUND OF THE INVENTION

Biofilms are communities of bacteria that are found on solid surfaces in many different environments, including surfaces of the oral cavity. Oral biofilm, or dental plaque, contains many of the bacteria that are associated with oral health issues such as oral malodor, demineralization, dental caries, tooth decay, potential loss of teeth and gum disease (gingivitis and periodontitis).

The formation of oral biofilm occurs in three stages known as the lag phase, growth phase, and steady state, respectively. In the lag phase, glycoproteins from saliva bind to an oral surface such as teeth and create a structure termed the pellicle that functions as attachment site for bacteria. In the growth phase, co-aggregation occurs, i.e., secondary bacterial colonizers attach to the primary bacterial colonizers, causing the diversity of the biofilm to increase and the biofilm to grow and mature. In the steady state, the biofilm growth slows down and eventually stops. This stage-based formation cycle causes biofilms to exist in several consecutive layers, which makes physical abrasion of biofilm more difficult.

Within a biofilm, the residing bacterial cells are distributed in an extracellular polymeric matrix that consists primarily of water, proteins, exopolysaccharides, lipopolysaccharides, lipids, surfactants, and extracellular DNA, with exopolysaccharides occupying a major fraction of the dry weight of biofilm (H. C. Flemming, and J. Wingender (2010), Nat. Rev. Microbiol. 8, 623-633). The exopolysaccharides are mainly glucose and fructose homopolymers, including (1-3)-α-D-glucans, (1-4)-α-D-glucans, (1-6)-α-D-glucans and (2-6)-β-D-fructans. These polysaccharides are synthesized from ingested sucrose by glucosyltransferases and fructosyltransferases secreted by oral bacteria such as Streptococcus spp., Lactobacillus spp., and Actinomyces spp.). Mutans and dextrans are particularly important glucans in the formation of dental plaque. Mutans have a highly branched structure with main chains composed of glucose molecules linked with (1-3)-α bonds and (1-6)-α-glycosidic linkages in their side chains. Dextrans are also high molecular weight polymers of glucose containing numerous consecutive (1-6)-α-linkages in their backbone and side chains, which begin from the (1-3)-α-linkage (M. Pleszczynska et al. (2016), Biotechnol. Appl. Biochem. 64(3), 337-346). Fructans are primarily linear polysaccharides and consist mainly of β-(2,6)-linked fructosyl residues and some β-(2,1)-linked branches.

Because of the increased resistance to anti-microbial agents as well as the mechanical properties of biofilm, many current oral care products are rather inefficient in addressing biofilm formation and alleviating the associated oral health issues. The focus for biofilm removal has been on mechanical abrasion. However, this approach is difficult due to the multilayered nature of biofilms and is further compromised by the fact that mechanical removal of biofilm, e.g., by brushing the teeth, expands and deepens the areas in the oral cavity where biofilms attach and expand, thus potentially increasing the severity of the problem rather than reducing it.

In view of the important role of biofilm in oral disease, there is a need in the art for oral care compositions that can effectively target oral biofilm. WO 1997/38669 (Novozymes) describes oral care compositions comprising a mutanase and a dextranase, WO 1998/57653 (Novozymes) provides oral care compositions comprising a dextranase and a pullulanase, WO 2000/17331 discloses oral care compositions comprising Paenibacillus fructanases, and WO 2020/099490 (Novozymes) describes oral care compositions comprising a mutanase and a DNase. However, there is a still need for oral care compositions targeting oral biofilm.

### SUMMARY OF THE INVENTION

The present invention provides oral care compositions comprising an invertase, a beta-glucosidase, and a glucoamylase useful for prevention of oral biofilm formation.

In a first aspect, the present invention relates to an oral care composition comprising an invertase, a beta-glucosidase, a glucoamylase, and at least one oral care ingredient. In a preferred embodiment of the first aspect, the oral care composition further comprises an alpha-amylase.

In a second aspect, the present invention relates to a composition according to the first aspect for use as a medicament.

In a third aspect, the present invention relates to a composition according to the first aspect for use in the treatment of oral disease.

In a fourth aspect, the present invention relates to a method for preventing or removing oral biofilm, the method comprising contacting the oral biofilm with an oral care composition according to the first aspect.

In a fifth aspect, the present invention relates to a method for reducing the risk of oral biofilm formation, the method comprising contacting the oral biofilm with an oral care composition according to the first aspect.

In a sixth aspect, the present invention relates to a kit of parts comprising:
a) an oral composition according to the first aspect; and
b) instructions for use.

### Definitions

In accordance with this detailed description, the following definitions apply. Note that the singular forms “a,” “an,” and “the” include plural references unless the context clearly dictates otherwise.

Unless defined otherwise or clearly indicated by context, all technical and scientific terms used herein have the same meaning as commonly understood by one of ordinary skill in the art to which this invention belongs.

Alpha-amylase: The term “alpha-amylase” means a polypeptide alpha-amylase activity that catalyzes the endohydrolysis of (1→4)-α-D-glucosidic linkages in polysaccharides containing three or more (1→4)-α-linked D-glucose units. The systematic name for alpha-amylase is 4-α-D-glucan glucanohydrolase (EC 3.2.1.1). The terms “alpha-amylase” and “α-amylase” and the expression “a polypeptide having alpha-amylase activity” are used interchangeably throughout this application. For purposes of the present invention, alpha-amylase activity may be determined according to Alpha-amylase activity assay I or Alpha-amylase activity assay II described in Example 2 below.

Beta-glucosidase: The term “beta-glucosidase” means a polypeptide having beta-glucosidase activity that catalyzes the hydrolysis of terminal non-reducing β-D-glucosyl residues with release of β-D-glucose. The systematic name for beta-glucosidase is β-D-glucoside glucohydrolase (EC 3.2.1.21). The terms “beta-glucosidase” and “β-glucosidase” and the expression “a polypeptide having beta-glucosidase activity” are used interchangeably throughout this application. For purposes of the present invention, beta-glucosidase activity may be determined according to the beta-glucosidase activity assay described in Example 2 below.

Biofilm prevention: The term “biofilm prevention” means the ability of a polypeptide to decrease the amount of a biofilm grown under defined conditions. For purposes of the present invention, biofilm prevention may be determined according to Example 4 or Example 5 herein.

Denture: The term “denture” is meant to cover dentures as such as well as braces, aligners, retainers, and the like.

Fragment: The term “fragment” means a polypeptide having one or more amino acids absent from the amino and/or carboxyl terminus of the mature polypeptide, wherein the fragment has the enzyme activity of mature polypeptide. A fragment of an invertase has invertase activity, a fragment of a beta-glucosidase has beta-glucosidase activity, a fragment of a glucoamylase has glucoamylase activity, and a fragment of an alpha-amylase has alpha-amylase activity.

Glucoamylase: The term “glucoamylase” means a polypeptide having glucoamylase activity that catalyzes the hydrolysis of terminal (1→4)-linked α-D-glucose residues successively from non-reducing ends of the chains with release of β-D-glucose. Glucoamylase is also known as glucan 1,4-α-glucosidase, and the systematic name for glucoamylase is 4-α-D-glucan glucohydrolase (EC 3.2.1.3). The term “glucoamylase” and the expression “a polypeptide having glucoamylase activity” are used interchangeably throughout this application. For purposes of the present invention, glucoamylase activity may be determined according to the glucoamylase activity assay described in Example 2 below.

Invertase: The term “invertase” means a polypeptide having invertase activity that catalyzes the hydrolysis of terminal non-reducing β-D-fructofuranoside residues in β-D-fructofuranosides. Invertase is also known as β-fructofuranosidase, and the systematic name for invertase is β-D-fructofuranoside fructohydrolase (EC 3.2.1.26). The term “invertase” and the expression “a polypeptide having invertase activity” are used interchangeably throughout this application. For purposes of the present invention, invertase activity may be determined according to the invertase activity assay described in Example 2 below.

Mature polypeptide: The term “mature polypeptide” means a polypeptide in its mature form following N-terminal and/or C-terminal processing (e.g., removal of signal peptide).

Parent: The term “parent” or means an enzyme to which an alteration is made to produce an enzyme variant. In one aspect, the parent is a parent invertase to which an alteration is made to produce an invertase variant. In one aspect, the parent is a parent beta-glucosidase to which an alteration is made to produce a beta-glucosidase variant. In one aspect, the parent is a parent glucoamylase to which an alteration is made to produce a glucoamylase variant. In one aspect, the parent is a parent alpha-amylase to which an alteration is made to produce an alpha-amylase variant.

Sequence identity: The relatedness between two amino acid sequences or between two nucleotide sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are a gap open penalty of 10, a gap extension penalty of 0.5, and the EBLOSUM62 (EMBOSS version of BLOSUM62) substitution matrix. For the Needle program to report the longest identity, the -nobrief option must be specified in the command line. The output of Needle labeled “longest identity” is calculated as follows:

(Identical Residues×100)/(Length of Alignment−Total Number of Gaps in Alignment)

For purposes of the present invention, the sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NUC4.4) substitution matrix. For the Needle program to report the longest identity, the nobrief option must be specified in the command line. The output of Needle labeled “longest identity” is calculated as follows:

(Identical Deoxyribonucleotides×100)/(Length of Alignment−Total Number of Gaps in Alignment)

Variant: The term “variant” means an invertase, a beta-glucosidase, a glucoamylase, or an alpha-amylase comprising a man-made mutation, i.e., a substitution, insertion (including extension), and/or deletion (e.g., truncation), at one or more positions. A substitution means replacement of the amino acid occupying a position with a different amino acid; a deletion means removal of the amino acid occupying a position; and an insertion means adding 1-5 amino acids (e.g., 1-3 amino acids, 1 amino acid) adjacent to and immediately following the amino acid occupying a position.

### BRIEF DESCRIPTION OF DRAWINGS

FIG. 1 shows an example of the thermal stability data generated using the nanoDSF instrument. Panel A is an example of the data obtained (the ratio of the fluorescence emission at 350 nm to 330 nm) in triplicate for SEQ ID NO:3 as a function of temperature. Panel B shows the first derivative of the raw data in Panel A. The peak maximum in the first derivative plot corresponds to the mid-point of the thermal unfolding transition, referred to as Tm. In this example the Tm corresponds to 61.9° C. at pH 6.0 for SEQ ID NO:3 and is highly reproducible within the three replicates.

### SEQUENCE OVERVIEW

SEQ ID NO:1 is the gDNA sequence of an invertase obtained from Aspergillus niger.
SEQ ID NO:2 is the translation product obtained from SEQ ID NO:2 (including the signal peptide).
SEQ ID NO:3 is an invertase obtained from Aspergillus niger(mature polypeptide).
SEQ ID NO:4 is the gDNA sequence of a beta-glucosidase obtained from Aspergillus niger.
SEQ ID NO:5 is the translation product obtained from SEQ ID NO:4 (including the signal peptide).
SEQ ID NO:6 is a beta-glucosidase obtained from Aspergillus niger(mature polypeptide).
SEQ ID NO:7 is the gDNA sequence of a glucoamylase obtained from Aspergillus niger.
SEQ ID NO:8 is the translation product obtained from SEQ ID NO:7 (including the signal peptide).
SEQ ID NO:9 is a glucoamylase obtained from Aspergillus niger(mature polypeptide).
SEQ ID NO:10 is the gDNA sequence of an invertase obtained from Bipolaris sorokiniana.
SEQ ID NO:11 is the translation product obtained from SEQ ID NO:10 (including the signal peptide).
SEQ ID NO:12 is an invertase obtained from Bipolaris sorokiniana (mature polypeptide).
SEQ ID NO:13 is the gDNA sequence of an invertase obtained from Aspergillus aculeatus.
SEQ ID NO:14 is the translation product obtained from SEQ ID NO:13 (including the signal peptide).
SEQ ID NO:15 is an invertase obtained from Aspergillus aculeatus (mature polypeptide).
SEQ ID NO:16 is the gDNA sequence of an invertase obtained from Pestalotiopsis vismiae.
SEQ ID NO:17 is the translation product obtained from SEQ ID NO:16 (including the signal peptide).
SEQ ID NO:18 is an invertase obtained from Pestalotiopsis vismiae (mature polypeptide).
SEQ ID NO:19 is the gDNA sequence of an invertase obtained from Aspergillus avenaceus.
SEQ ID NO:20 is the translation product obtained from SEQ ID NO:19 (including the signal peptide).
SEQ ID NO:21 is an invertase obtained from Aspergillus avenaceus (mature polypeptide).
SEQ ID NO:22 is the gDNA sequence of an invertase obtained from Aspergillus sclerotiorum.
SEQ ID NO:23 is the translation product obtained from SEQ ID NO:22 (including the signal peptide).
SEQ ID NO:24 is an invertase obtained from Aspergillus sclerotiorum (mature polypeptide).
SEQ ID NO:25 is the gDNA sequence of an invertase obtained from Fusarium avenaceum.
SEQ ID NO:26 is the translation product obtained from SEQ ID NO:25 (including the signal peptide).
SEQ ID NO:27 is an invertase obtained from Fusarium avenaceum (mature polypeptide).
SEQ ID NO:28 is the gDNA sequence of an invertase obtained from Penicillium coprophilum.
SEQ ID NO:29 is the translation product obtained from SEQ ID NO:28 (including the signal peptide).
SEQ ID NO:30 is an invertase obtained from Penicillium coprophilum (mature polypeptide).
SEQ ID NO:31 is the gDNA sequence of an invertase obtained from Penicillium murcianum.
SEQ ID NO:32 is the translation product obtained from SEQ ID NO:31 (including the signal peptide).
SEQ ID NO:33 is an invertase obtained from Penicillium murcianum (mature polypeptide).
SEQ ID NO:34 is the gDNA sequence of an invertase obtained from Penicillium venetum.
SEQ ID NO:35 is the translation product obtained from SEQ ID NO:34 (including the signal peptide).
SEQ ID NO:36 is an invertase obtained from Penicillium venetum (mature polypeptide).
SEQ ID NO:37 is the gDNA sequence of an invertase obtained from Curvularia spicifera.
SEQ ID NO:38 is the translation product obtained from SEQ ID NO:37 (including the signal peptide).
SEQ ID NO:39 is an invertase obtained from Curvularia spicifera (mature polypeptide).
SEQ ID NO:40 is the gDNA sequence of an invertase obtained from Alternaria sp.
SEQ ID NO:41 is the translation product obtained from SEQ ID NO:40 (including the signal peptide).
SEQ ID NO:42 is an invertase obtained from Alternaria sp. (mature polypeptide).
SEQ ID NO:43 is the gDNA sequence of an invertase obtained from Fusarium temperatum.
SEQ ID NO:44 is the translation product obtained from SEQ ID NO:43 (including the signal peptide).
SEQ ID NO:45 is an invertase obtained from Fusarium temperatum (mature polypeptide).
SEQ ID NO:46 is the gDNA sequence of an invertase obtained from Aspergillus japonicus.
SEQ ID NO:47 is the translation product obtained from SEQ ID NO:46 (including the signal 10 peptide).
SEQ ID NO:48 is an invertase obtained from Aspergillus japonicus (mature polypeptide).
SEQ ID NO:49 is the coding sequence of an alpha-amylase obtained from Bacillus amyloliquefaciens.
SEQ ID NO:50 is an alpha-amylase obtained from Bacillus amyloliquefaciens (mature polypeptide).
SEQ ID NO:51 is a secretion signal from Bacillus licheniformis.
SEQ ID NO:52 is a His affinity tag.

### DETAILED DESCRIPTION OF THE INVENTION

The present invention relates to oral care compositions comprising an invertase, a beta-glucosidase, a glucoamylase, and at least one oral care ingredient. As illustrated in the Examples of the present application, the present inventors have identified,

invertases, beta-glucosidases, and glycoamylases, in particular those of microbial origin, as being very effective in preventing formation of oral biofilm and/or reducing the risk of oral biofilm formation. In addition, these enzymes are highly stabile in the presence of a wide range oral care ingredients, making them very suitable for oral care formulations. Without being bound by theory, it is speculated that the combination of invertase, beta-glucosidase, and glucoamylase is particularly effective in degrading the exopolysaccharides found in oral biofilm as well as the dietary carbohydrates that functions as substrate for oral bacteria, and that this provides the preventive effect on oral biofilm formation.

The present inventors have further identified that the combination of an invertase, a beta-glucosidase, a glucoamylase, and an alpha-amylase is able to potently remove biofilm already present in the oral cavity.

Thus, in a first aspect, the present invention relates to oral care compositions comprising an invertase, a beta-glucosidase, a glucoamylase, and at least one oral care ingredient. In a preferred embodiment, the oral care compositions further comprise an alpha-amylase.

### Invertases

The invertase may be derived from any organism. Preferably, the invertase is of microbial origin; most preferably, the invertase is of bacterial or fungal origin. In a preferred embodiment, the invertase is derived from Aspergillus niger. In a preferred embodiment, the invertase is derived from Bipolaris sorokiniana. In a preferred embodiment, the invertase is derived from Aspergillus aculeatus. In a preferred embodiment, the invertase is derived from Pestalotiopsis vismiae. In a preferred embodiment, the invertase is derived from Aspergillus avenaceus. In a preferred embodiment, the invertase is derived from Aspergillus sclerotiorum. In a preferred embodiment, the invertase is derived from Fusarium avenaceum. The invertase may be derived from any organism. In a preferred embodiment, the invertase is derived from Penicillium coprophilum. In a preferred embodiment, the invertase is derived from Penicillium murcianum. In a preferred embodiment, the invertase is derived from Penicillium venetum. In a preferred embodiment, the invertase is derived from Curvularia spicifera. In a preferred embodiment, the invertase is derived from Alternaria sp. In a preferred embodiment, the invertase is derived from Fusarium temperatum. In a preferred embodiment, the invertase is derived from Aspergillus japonicus.

In one aspect, the invertase is selected from the group consisting of:
(a) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:2;
(b) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:3;
c) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:2;
(d) a polypeptide encoded by a polynucleotide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to the mature polypeptide coding sequence of SEQ ID NO:1 or the cDNA sequence thereof;
(e) a polypeptide derived from SEQ ID NO:2, a mature polypeptide of SEQ ID NO:2, or SEQ ID NO:3 by substitution, deletion, or addition of one or several amino acids;
(f) a polypeptide derived from the polypeptide of (a), (b), (c), (d) or (e) wherein the N- and/or C-terminal end has been extended by the addition of one or more amino acids; and
(g) a fragment of the polypeptide of (a), (b), (c), (d), or (e); wherein the polypeptide has invertase activity.

In a preferred embodiment, the invertase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:2 or a mature polypeptide of SEQ ID NO:2. A preferred mature polypeptide of SEQ ID NO:2 corresponds to amino acid residues 16 to 628 of SEQ ID NO:2.

In a preferred embodiment, the invertase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:3.

In a preferred embodiment, the invertase comprises, consists essentially of, or consists of SEQ ID NO:2 or a mature polypeptide of SEQ ID NO:2. A preferred mature polypeptide of SEQ ID NO:2 corresponds to amino acid residues 16 to 628 of SEQ ID NO:2.

In a preferred embodiment, the invertase comprises, consists essentially of, or consists of SEQ ID NO:3 or a fragment thereof.

The invertase may have an N-terminal and/or C-terminal extension of one or more amino acids, e.g., 1-5 amino acids.

In another aspect, the invertase is derived from SEQ ID NO:2 by substitution, deletion, or addition of one or several amino acids. In another aspect, the polypeptide is derived from a mature polypeptide of SEQ ID NO:2 by substitution, deletion, or addition of one or several amino acids.

In another aspect, the polypeptide is derived from SEQ ID NO:3 by substitution, deletion, or addition of one or more amino acids.

In some embodiments, the invertase is a variant of parent invertase, preferably SEQ ID NO:3, comprising a substitution, deletion, and/or insertion at one or more positions. In one aspect, the invertase is a variant of SEQ ID NO:3 and the number of amino acid substitutions, deletions and/or insertions introduced into the polypeptide of SEQ ID NO:3 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

In one aspect, the invertase is selected from the group consisting of:
(a) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:11;
(b) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:12;
c) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:11;
(d) a polypeptide encoded by a polynucleotide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to the mature polypeptide coding sequence of SEQ ID NO:10 or the cDNA sequence thereof;
(e) a polypeptide derived from SEQ ID NO:11, a mature polypeptide of SEQ ID NO:11, or SEQ ID NO:12 by substitution, deletion, or addition of one or several amino acids;
(f) a polypeptide derived from the polypeptide of (a), (b), (c), (d) or (e) wherein the N- and/or C-terminal end has been extended by the addition of one or more amino acids; and
(g) a fragment of the polypeptide of (a), (b), (c), (d), or (e); wherein the polypeptide has invertase activity.

In a preferred embodiment, the invertase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:11 or a mature polypeptide of SEQ ID NO:11. A preferred mature polypeptide of SEQ ID NO:11 corresponds to amino acid residues 22 to 637 of SEQ ID NO:11.

In a preferred embodiment, the invertase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:12.

In a preferred embodiment, the invertase comprises, consists essentially of, or consists of SEQ ID NO:11 or a mature polypeptide of SEQ ID NO:11. A preferred mature polypeptide of SEQ ID NO:11 corresponds to amino acid residues 22 to 637 of SEQ ID NO:11.

In a preferred embodiment, the invertase comprises, consists essentially of, or consists of SEQ ID NO:12 or a fragment thereof.

The invertase may have an N-terminal and/or C-terminal extension of one or more amino acids, e.g., 1-5 amino acids.

In another aspect, the invertase is derived from SEQ ID NO: 11 by substitution, deletion, or addition of one or several amino acids. In another aspect, the polypeptide is derived from a mature polypeptide of SEQ ID NO: 11 by substitution, deletion, or addition of one or several amino acids.

In another aspect, the polypeptide is derived from SEQ ID NO:12 by substitution, deletion, or addition of one or more amino acids.

In some embodiments, the invertase is a variant of parent invertase, preferably SEQ ID NO:12, comprising a substitution, deletion, and/or insertion at one or more positions. In one aspect, the invertase is a variant of SEQ ID NO:12 and the number of amino acid substitutions, deletions and/or insertions introduced into the polypeptide of SEQ ID NO:12 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

In one aspect, the invertase is selected from the group consisting of:
(a) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:14;
(b) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:15;
c) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:14;
(d) a polypeptide encoded by a polynucleotide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to the mature polypeptide coding sequence of SEQ ID NO:13 or the cDNA sequence thereof;
(e) a polypeptide derived from SEQ ID NO:14, a mature polypeptide of SEQ ID NO:14, or SEQ ID NO:15 by substitution, deletion, or addition of one or several amino acids;
(f) a polypeptide derived from the polypeptide of (a), (b), (c), (d) or (e) wherein the N- and/or C-terminal end has been extended by the addition of one or more amino acids; and
(g) a fragment of the polypeptide of (a), (b), (c), (d), or (e); wherein the polypeptide has invertase activity.

In a preferred embodiment, the invertase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:14 or a mature polypeptide of SEQ ID NO:14. A preferred mature polypeptide of SEQ ID NO:14 corresponds to amino acid residues 17 to 651 of SEQ ID NO:14.

In a preferred embodiment, the invertase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:15.

In a preferred embodiment, the invertase comprises, consists essentially of, or consists of SEQ ID NO:14 or a mature polypeptide of SEQ ID NO:14. A preferred mature polypeptide of SEQ ID NO:14 corresponds to amino acid residues 17 to 651 of SEQ ID NO:14.

In a preferred embodiment, the invertase comprises, consists essentially of, or consists of SEQ ID NO:15 or a fragment thereof.

The invertase may have an N-terminal and/or C-terminal extension of one or more amino acids, e.g., 1-5 amino acids.

In another aspect, the invertase is derived from SEQ ID NO:14 by substitution, deletion, or addition of one or several amino acids. In another aspect, the polypeptide is derived from a mature polypeptide of SEQ ID NO:14 by substitution, deletion, or addition of one or several amino acids.

In another aspect, the polypeptide is derived from SEQ ID NO:15 by substitution, deletion, or addition of one or more amino acids.

In some embodiments, the invertase is a variant of parent invertase, preferably SEQ ID NO:15, comprising a substitution, deletion, and/or insertion at one or more positions. In one aspect, the invertase is a variant of SEQ ID NO:15 and the number of amino acid substitutions, deletions and/or insertions introduced into the polypeptide of SEQ ID NO:15 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

In one aspect, the invertase is selected from the group consisting of:
(a) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:17;
(b) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:18;
c) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:17;
(d) a polypeptide encoded by a polynucleotide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to the mature polypeptide coding sequence of SEQ ID NO:16 or the cDNA sequence thereof;
(e) a polypeptide derived from SEQ ID NO:17, a mature polypeptide of SEQ ID NO:17, or SEQ ID NO:18 by substitution, deletion, or addition of one or several amino acids;
(f) a polypeptide derived from the polypeptide of (a), (b), (c), (d) or (e) wherein the N- and/or C-terminal end has been extended by the addition of one or more amino acids; and
(g) a fragment of the polypeptide of (a), (b), (c), (d), or (e); wherein the polypeptide has invertase activity.

In a preferred embodiment, the invertase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:17 or a mature polypeptide of SEQ ID NO:17. A preferred mature polypeptide of SEQ ID NO:17 corresponds to amino acid residues 21 to 621 of SEQ ID NO:17.

In a preferred embodiment, the invertase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:18.

In a preferred embodiment, the invertase comprises, consists essentially of, or consists of SEQ ID NO:17 or a mature polypeptide of SEQ ID NO:17. A preferred mature polypeptide of SEQ ID NO:17 corresponds to amino acid residues 21 to 621 of SEQ ID NO:17.

In a preferred embodiment, the invertase comprises, consists essentially of, or consists of SEQ ID NO:18 or a fragment thereof.

The invertase may have an N-terminal and/or C-terminal extension of one or more amino acids, e.g., 1-5 amino acids.

In another aspect, the invertase is derived from SEQ ID NO:17 by substitution, deletion, or addition of one or several amino acids. In another aspect, the polypeptide is derived from a mature polypeptide of SEQ ID NO:17 by substitution, deletion, or addition of one or several amino acids.

In another aspect, the polypeptide is derived from SEQ ID NO:18 by substitution, deletion, or addition of one or more amino acids.

In some embodiments, the invertase is a variant of parent invertase, preferably SEQ ID NO:18, comprising a substitution, deletion, and/or insertion at one or more positions. In one aspect, the invertase is a variant of SEQ ID NO:18 and the number of amino acid substitutions, deletions and/or insertions introduced into the polypeptide of SEQ ID NO:18 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

In one aspect, the invertase is selected from the group consisting of:
(a) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:20;
(b) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:21;
c) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:20;
(d) a polypeptide encoded by a polynucleotide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to the mature polypeptide coding sequence of SEQ ID NO:19 or the cDNA sequence thereof;
(e) a polypeptide derived from SEQ ID NO:20, a mature polypeptide of SEQ ID NO:20, or SEQ ID NO:21 by substitution, deletion, or addition of one or several amino acids;
(f) a polypeptide derived from the polypeptide of (a), (b), (c), (d) or (e) wherein the N- and/or C-terminal end has been extended by the addition of one or more amino acids; and
(g) a fragment of the polypeptide of (a), (b), (c), (d), or (e); wherein the polypeptide has invertase activity.

In a preferred embodiment, the invertase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:20 or a mature polypeptide of SEQ ID NO:20. A preferred mature polypeptide of SEQ ID NO:20 corresponds to amino acid residues 19 to 624 of SEQ ID NO:20.

In a preferred embodiment, the invertase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:21.

In a preferred embodiment, the invertase comprises, consists essentially of, or consists of SEQ ID NO:20 or a mature polypeptide of SEQ ID NO:20. A preferred mature polypeptide of SEQ ID NO:20 corresponds to amino acid residues 19 to 624 of SEQ ID NO:20.

In a preferred embodiment, the invertase comprises, consists essentially of, or consists of SEQ ID NO:21 or a fragment thereof.

The invertase may have an N-terminal and/or C-terminal extension of one or more amino acids, e.g., 1-5 amino acids.

In another aspect, the invertase is derived from SEQ ID NO:20 by substitution, deletion, or addition of one or several amino acids. In another aspect, the polypeptide is derived from a mature polypeptide of SEQ ID NO:20 by substitution, deletion, or addition of one or several amino acids.

In another aspect, the polypeptide is derived from SEQ ID NO:21 by substitution, deletion, or addition of one or more amino acids.

In some embodiments, the invertase is a variant of parent invertase, preferably SEQ ID NO:21, comprising a substitution, deletion, and/or insertion at one or more positions. In one aspect, the invertase is a variant of SEQ ID NO:21 and the number of amino acid substitutions, deletions and/or insertions introduced into the polypeptide of SEQ ID NO:21 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

In one aspect, the invertase is selected from the group consisting of:
(a) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:23;
(b) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:24;
c) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:23;
(d) a polypeptide encoded by a polynucleotide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to the mature polypeptide coding sequence of SEQ ID NO:22 or the cDNA sequence thereof;
(e) a polypeptide derived from SEQ ID NO:23, a mature polypeptide of SEQ ID NO:23, or SEQ ID NO:24 by substitution, deletion, or addition of one or several amino acids;
(f) a polypeptide derived from the polypeptide of (a), (b), (c), (d) or (e) wherein the N- and/or C-terminal end has been extended by the addition of one or more amino acids; and
(g) a fragment of the polypeptide of (a), (b), (c), (d), or (e); wherein the polypeptide has invertase activity.

In a preferred embodiment, the invertase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:23 or a mature polypeptide of SEQ ID NO:23. A preferred mature polypeptide of SEQ ID NO:23 corresponds to amino acid residues 19 to 621 of SEQ ID NO:23.

In a preferred embodiment, the invertase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:24.

In a preferred embodiment, the invertase comprises, consists essentially of, or consists of SEQ ID NO:23 or a mature polypeptide of SEQ ID NO:23. A preferred mature polypeptide of SEQ ID NO:23 corresponds to amino acid residues 19 to 621 of SEQ ID NO:23.

In a preferred embodiment, the invertase comprises, consists essentially of, or consists of SEQ ID NO:24 or a fragment thereof.

The invertase may have an N-terminal and/or C-terminal extension of one or more amino acids, e.g., 1-5 amino acids.

In another aspect, the invertase is derived from SEQ ID NO:23 by substitution, deletion, or addition of one or several amino acids. In another aspect, the polypeptide is derived from a mature polypeptide of SEQ ID NO:23 by substitution, deletion, or addition of one or several amino acids.

In another aspect, the polypeptide is derived from SEQ ID NO:24 by substitution, deletion, or addition of one or more amino acids.

In some embodiments, the invertase is a variant of parent invertase, preferably SEQ ID NO:24, comprising a substitution, deletion, and/or insertion at one or more positions. In one aspect, the invertase is a variant of SEQ ID NO:24 and the number of amino acid substitutions, deletions and/or insertions introduced into the polypeptide of SEQ ID NO:24 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

In one aspect, the invertase is selected from the group consisting of:
(a) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:26;
(b) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:27;
c) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:26;
(d) a polypeptide encoded by a polynucleotide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to the mature polypeptide coding sequence of SEQ ID NO:25 or the cDNA sequence thereof;
(e) a polypeptide derived from SEQ ID NO:26, a mature polypeptide of SEQ ID NO:26, or SEQ ID NO:27 by substitution, deletion, or addition of one or several amino acids;
(f) a polypeptide derived from the polypeptide of (a), (b), (c), (d) or (e) wherein the N- and/or C-terminal end has been extended by the addition of one or more amino acids; and
(g) a fragment of the polypeptide of (a), (b), (c), (d), or (e); wherein the polypeptide has invertase activity.

In a preferred embodiment, the invertase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:26 or a mature polypeptide of SEQ ID NO:26. A preferred mature polypeptide of SEQ ID NO:26 corresponds to amino acid residues 16 to 619 of SEQ ID NO:26.

In a preferred embodiment, the invertase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:27.

In a preferred embodiment, the invertase comprises, consists essentially of, or consists of SEQ ID NO:26 or a mature polypeptide of SEQ ID NO:26. A preferred mature polypeptide of SEQ ID NO:26 corresponds to amino acid residues 16 to 619 of SEQ ID NO:26.

In a preferred embodiment, the invertase comprises, consists essentially of, or consists of SEQ ID NO:27 or a fragment thereof.

The invertase may have an N-terminal and/or C-terminal extension of one or more amino acids, e.g., 1-5 amino acids.

In another aspect, the invertase is derived from SEQ ID NO:26 by substitution, deletion, or addition of one or several amino acids. In another aspect, the polypeptide is derived from a mature polypeptide of SEQ ID NO:26 by substitution, deletion, or addition of one or several amino acids.

In another aspect, the polypeptide is derived from SEQ ID NO:27 by substitution, deletion, or addition of one or more amino acids.

In some embodiments, the invertase is a variant of parent invertase, preferably SEQ ID NO:27, comprising a substitution, deletion, and/or insertion at one or more positions. In one aspect, the invertase is a variant of SEQ ID NO:27 and the number of amino acid substitutions, deletions and/or insertions introduced into the polypeptide of SEQ ID NO:27 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

In one aspect, the invertase is selected from the group consisting of:
(a) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:29;
(b) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:30;
c) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:29;
(d) a polypeptide encoded by a polynucleotide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to the mature polypeptide coding sequence of SEQ ID NO:28 or the cDNA sequence thereof;
(e) a polypeptide derived from SEQ ID NO:29, a mature polypeptide of SEQ ID NO:29, or SEQ ID NO:30 by substitution, deletion, or addition of one or several amino acids;
(f) a polypeptide derived from the polypeptide of (a), (b), (c), (d) or (e) wherein the N- and/or C-terminal end has been extended by the addition of one or more amino acids; and
(g) a fragment of the polypeptide of (a), (b), (c), (d), or (e); wherein the polypeptide has invertase activity.

In a preferred embodiment, the invertase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:29 or a mature polypeptide of SEQ ID NO:29. A preferred mature polypeptide of SEQ ID NO:29 corresponds to amino acid residues 18 to 620 of SEQ ID NO:29.

In a preferred embodiment, the invertase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:30.

In a preferred embodiment, the invertase comprises, consists essentially of, or consists of SEQ ID NO:29 or a mature polypeptide of SEQ ID NO:29. A preferred mature polypeptide of SEQ ID NO:29 corresponds to amino acid residues 18 to 620 of SEQ ID NO:29.

In a preferred embodiment, the invertase comprises, consists essentially of, or consists of SEQ ID NO:30 or a fragment thereof.

The invertase may have an N-terminal and/or C-terminal extension of one or more amino acids, e.g., 1-5 amino acids.

In another aspect, the invertase is derived from SEQ ID NO:29 by substitution, deletion, or addition of one or several amino acids. In another aspect, the polypeptide is derived from a mature polypeptide of SEQ ID NO:29 by substitution, deletion, or addition of one or several amino acids.

In another aspect, the polypeptide is derived from SEQ ID NO:30 by substitution, deletion, or addition of one or more amino acids.

In some embodiments, the invertase is a variant of parent invertase, preferably SEQ ID NO:30, comprising a substitution, deletion, and/or insertion at one or more positions. In one aspect, the invertase is a variant of SEQ ID NO:30 and the number of amino acid substitutions, deletions and/or insertions introduced into the polypeptide of SEQ ID NO:30 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

In one aspect, the invertase is selected from the group consisting of:
(a) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:32;
(b) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:33;
c) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:32;
(d) a polypeptide encoded by a polynucleotide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to the mature polypeptide coding sequence of SEQ ID NO:31 or the cDNA sequence thereof;
(e) a polypeptide derived from SEQ ID NO:32, a mature polypeptide of SEQ ID NO:32, or SEQ ID NO:33 by substitution, deletion, or addition of one or several amino acids;
(f) a polypeptide derived from the polypeptide of (a), (b), (c), (d) or (e) wherein the N- and/or C-terminal end has been extended by the addition of one or more amino acids; and
(g) a fragment of the polypeptide of (a), (b), (c), (d), or (e); wherein the polypeptide has invertase activity.

In a preferred embodiment, the invertase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:32 or a mature polypeptide of SEQ ID NO:32. A preferred mature polypeptide of SEQ ID NO:32 corresponds to amino acid residues 18 to 623 of SEQ ID NO:32.

In a preferred embodiment, the invertase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:33.

In a preferred embodiment, the invertase comprises, consists essentially of, or consists of SEQ ID NO:32 or a mature polypeptide of SEQ ID NO:32. A preferred mature polypeptide of SEQ ID NO:32 corresponds to amino acid residues 18 to 623 of SEQ ID NO:32.

In a preferred embodiment, the invertase comprises, consists essentially of, or consists of SEQ ID NO:33 or a fragment thereof.

The invertase may have an N-terminal and/or C-terminal extension of one or more amino acids, e.g., 1-5 amino acids.

In another aspect, the invertase is derived from SEQ ID NO:32 by substitution, deletion, or addition of one or several amino acids. In another aspect, the polypeptide is derived from a mature polypeptide of SEQ ID NO:32 by substitution, deletion, or addition of one or several amino acids.

In another aspect, the polypeptide is derived from SEQ ID NO:33 by substitution, deletion, or addition of one or more amino acids.

In some embodiments, the invertase is a variant of parent invertase, preferably SEQ ID NO:33, comprising a substitution, deletion, and/or insertion at one or more positions. In one aspect, the invertase is a variant of SEQ ID NO:33 and the number of amino acid substitutions, deletions and/or insertions introduced into the polypeptide of SEQ ID NO:33 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

In one aspect, the invertase is selected from the group consisting of:
(a) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:35;
(b) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:36;
c) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:35;
(d) a polypeptide encoded by a polynucleotide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to the mature polypeptide coding sequence of SEQ ID NO:34 or the cDNA sequence thereof;
(e) a polypeptide derived from SEQ ID NO:35, a mature polypeptide of SEQ ID NO:35, or SEQ ID NO:36 by substitution, deletion, or addition of one or several amino acids;
(f) a polypeptide derived from the polypeptide of (a), (b), (c), (d) or (e) wherein the N- and/or C-terminal end has been extended by the addition of one or more amino acids; and
(g) a fragment of the polypeptide of (a), (b), (c), (d), or (e); wherein the polypeptide has invertase activity.

In a preferred embodiment, the invertase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:35 or a mature polypeptide of SEQ ID NO:35. A preferred mature polypeptide of SEQ ID NO:35 corresponds to amino acid residues 18 to 621 of SEQ ID NO:35.

In a preferred embodiment, the invertase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:36.

In a preferred embodiment, the invertase comprises, consists essentially of, or consists of SEQ ID NO:35 or a mature polypeptide of SEQ ID NO:35. A preferred mature polypeptide of SEQ ID NO:35 corresponds to amino acid residues 18 to 621 of SEQ ID NO:35.

In a preferred embodiment, the invertase comprises, consists essentially of, or consists of SEQ ID NO:36 or a fragment thereof.

The invertase may have an N-terminal and/or C-terminal extension of one or more amino acids, e.g., 1-5 amino acids.

In another aspect, the invertase is derived from SEQ ID NO:35 by substitution, deletion, or addition of one or several amino acids. In another aspect, the polypeptide is derived from a mature polypeptide of SEQ ID NO:35 by substitution, deletion, or addition of one or several amino acids.

In another aspect, the polypeptide is derived from SEQ ID NO:36 by substitution, deletion, or addition of one or more amino acids.

In some embodiments, the invertase is a variant of parent invertase, preferably SEQ ID NO:36, comprising a substitution, deletion, and/or insertion at one or more positions. In one aspect, the invertase is a variant of SEQ ID NO:36 and the number of amino acid substitutions, deletions and/or insertions introduced into the polypeptide of SEQ ID NO:36 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

In one aspect, the invertase is selected from the group consisting of:
(a) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:38;
(b) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:39;
c) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:38;
(d) a polypeptide encoded by a polynucleotide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to the mature polypeptide coding sequence of SEQ ID NO:37 or the cDNA sequence thereof;
(e) a polypeptide derived from SEQ ID NO:38, a mature polypeptide of SEQ ID NO:38, or SEQ ID NO:39 by substitution, deletion, or addition of one or several amino acids;
(f) a polypeptide derived from the polypeptide of (a), (b), (c), (d) or (e) wherein the N- and/or C-terminal end has been extended by the addition of one or more amino acids; and
(g) a fragment of the polypeptide of (a), (b), (c), (d), or (e); wherein the polypeptide has invertase activity.

In a preferred embodiment, the invertase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:38 or a mature polypeptide of SEQ ID NO:38. A preferred mature polypeptide of SEQ ID NO:38 corresponds to amino acid residues 24 to 637 of SEQ ID NO:38.

In a preferred embodiment, the invertase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:39.

In a preferred embodiment, the invertase comprises, consists essentially of, or consists of SEQ ID NO:38 or a mature polypeptide of SEQ ID NO:38. A preferred mature polypeptide of SEQ ID NO:38 corresponds to amino acid residues 24 to 637 of SEQ ID NO:38.

In a preferred embodiment, the invertase comprises, consists essentially of, or consists of SEQ ID NO:39 or a fragment thereof.

The invertase may have an N-terminal and/or C-terminal extension of one or more amino acids, e.g., 1-5 amino acids.

In another aspect, the invertase is derived from SEQ ID NO:38 by substitution, deletion, or addition of one or several amino acids. In another aspect, the polypeptide is derived from a mature polypeptide of SEQ ID NO:38 by substitution, deletion, or addition of one or several amino acids.

In another aspect, the polypeptide is derived from SEQ ID NO:39 by substitution, deletion, or addition of one or more amino acids.

In some embodiments, the invertase is a variant of parent invertase, preferably SEQ ID NO:39, comprising a substitution, deletion, and/or insertion at one or more positions. In one aspect, the invertase is a variant of SEQ ID NO:39 and the number of amino acid substitutions, deletions and/or insertions introduced into the polypeptide of SEQ ID NO:39 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

In one aspect, the invertase is selected from the group consisting of:
(a) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:41;
(b) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:42;
c) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:41;
(d) a polypeptide encoded by a polynucleotide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to the mature polypeptide coding sequence of SEQ ID NO:40 or the cDNA sequence thereof;
(e) a polypeptide derived from SEQ ID NO:41, a mature polypeptide of SEQ ID NO:41, or 25 SEQ ID NO:42 by substitution, deletion, or addition of one or several amino acids;
(f) a polypeptide derived from the polypeptide of (a), (b), (c), (d) or (e) wherein the N- and/or C-terminal end has been extended by the addition of one or more amino acids; and
(g) a fragment of the polypeptide of (a), (b), (c), (d), or (e); wherein the polypeptide has invertase activity.

In a preferred embodiment, the invertase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:41 or a mature polypeptide of SEQ ID NO:41. A preferred mature polypeptide of SEQ ID NO:41 corresponds to amino acid residues 23 to 626 of SEQ ID NO:41.

In a preferred embodiment, the invertase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:42.

In a preferred embodiment, the invertase comprises, consists essentially of, or consists of SEQ ID NO:41 or a mature polypeptide of SEQ ID NO:41. A preferred mature polypeptide of SEQ ID NO:41 corresponds to amino acid residues 23 to 626 of SEQ ID NO:41.

In a preferred embodiment, the invertase comprises, consists essentially of, or consists of SEQ ID NO:42 or a fragment thereof.

The invertase may have an N-terminal and/or C-terminal extension of one or more amino acids, e.g., 1-5 amino acids.

In another aspect, the invertase is derived from SEQ ID NO:41 by substitution, deletion, or addition of one or several amino acids. In another aspect, the polypeptide is derived from a mature polypeptide of SEQ ID NO:41 by substitution, deletion, or addition of one or several amino acids.

In another aspect, the polypeptide is derived from SEQ ID NO:42 by substitution, deletion, or addition of one or more amino acids.

In some embodiments, the invertase is a variant of parent invertase, preferably SEQ ID 20 NO:42, comprising a substitution, deletion, and/or insertion at one or more positions. In one aspect, the invertase is a variant of SEQ ID NO:42 and the number of amino acid substitutions, deletions and/or insertions introduced into the polypeptide of SEQ ID NO:42 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

In one aspect, the invertase is selected from the group consisting of:
(a) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:44;
(b) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:45;
c) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:44;
(d) a polypeptide encoded by a polynucleotide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to the mature polypeptide coding sequence of SEQ ID NO:43 or the cDNA sequence thereof;
(e) a polypeptide derived from SEQ ID NO:44, a mature polypeptide of SEQ ID NO:44, or SEQ ID NO:45 by substitution, deletion, or addition of one or several amino acids;
(f) a polypeptide derived from the polypeptide of (a), (b), (c), (d) or (e) wherein the N- and/or C-terminal end has been extended by the addition of one or more amino acids; and
(g) a fragment of the polypeptide of (a), (b), (c), (d), or (e); wherein the polypeptide has invertase activity.

In a preferred embodiment, the invertase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:44 or a mature polypeptide of SEQ ID NO:44. A preferred mature polypeptide of SEQ ID NO:44 corresponds to amino acid residues 16 to 618 of SEQ ID NO:44.

In a preferred embodiment, the invertase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:45.

In a preferred embodiment, the invertase comprises, consists essentially of, or consists of SEQ ID NO:44 or a mature polypeptide of SEQ ID NO:44. A preferred mature polypeptide of SEQ ID NO:44 corresponds to amino acid residues 16 to 618 of SEQ ID NO:44.

In a preferred embodiment, the invertase comprises, consists essentially of, or consists of SEQ ID NO:45 or a fragment thereof.

The invertase may have an N-terminal and/or C-terminal extension of one or more amino acids, e.g., 1-5 amino acids.

In another aspect, the invertase is derived from SEQ ID NO:44 by substitution, deletion, or addition of one or several amino acids. In another aspect, the polypeptide is derived from a mature polypeptide of SEQ ID NO:44 by substitution, deletion, or addition of one or several amino acids.

In another aspect, the polypeptide is derived from SEQ ID NO:45 by substitution, deletion, or addition of one or more amino acids.

In some embodiments, the invertase is a variant of parent invertase, preferably SEQ ID NO:45, comprising a substitution, deletion, and/or insertion at one or more positions. In one aspect, the invertase is a variant of SEQ ID NO:45 and the number of amino acid substitutions, deletions and/or insertions introduced into the polypeptide of SEQ ID NO:45 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

In one aspect, the invertase is selected from the group consisting of:
(a) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:47;
(b) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:48;
c) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:47;
(d) a polypeptide encoded by a polynucleotide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to the mature polypeptide coding sequence of SEQ ID NO:46 or the cDNA sequence thereof;
(e) a polypeptide derived from SEQ ID NO:47, a mature polypeptide of SEQ ID NO:47, or SEQ ID NO:48 by substitution, deletion, or addition of one or several amino acids;
(f) a polypeptide derived from the polypeptide of (a), (b), (c), (d) or (e) wherein the N- and/or C-terminal end has been extended by the addition of one or more amino acids; and
(g) a fragment of the polypeptide of (a), (b), (c), (d), or (e); wherein the polypeptide has invertase activity.

In a preferred embodiment, the invertase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:47 or a mature polypeptide of SEQ ID NO:47. A preferred mature polypeptide of SEQ ID NO:47 corresponds to amino acid residues 20 to 653 of SEQ ID NO:47.

In a preferred embodiment, the invertase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:48.

In a preferred embodiment, the invertase comprises, consists essentially of, or consists of SEQ ID NO:47 or a mature polypeptide of SEQ ID NO:47. A preferred mature polypeptide of SEQ ID NO:47 corresponds to amino acid residues 20 to 653 of SEQ ID NO:47.

In a preferred embodiment, the invertase comprises, consists essentially of, or consists of SEQ ID NO:48 or a fragment thereof.

The invertase may have an N-terminal and/or C-terminal extension of one or more amino acids, e.g., 1-5 amino acids.

In another aspect, the invertase is derived from SEQ ID NO:47 by substitution, deletion, or addition of one or several amino acids. In another aspect, the polypeptide is derived from a mature polypeptide of SEQ ID NO:47 by substitution, deletion, or addition of one or several amino acids.

In another aspect, the polypeptide is derived from SEQ ID NO:48 by substitution, deletion, or addition of one or more amino acids.

In some embodiments, the invertase is a variant of parent invertase, preferably SEQ ID NO:48, comprising a substitution, deletion, and/or insertion at one or more positions. In one aspect, the invertase is a variant of SEQ ID NO:48 and the number of amino acid substitutions, deletions and/or insertions introduced into the polypeptide of SEQ ID NO:48 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant molecules are tested for invertase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide, and/or be inferred from sequence homology and conserved catalytic machinery with a related polypeptide or within a polypeptide or protein family with polypeptides/proteins descending from a common ancestor, typically having similar three-dimensional structures, functions, and significant sequence similarity. Additionally, or alternatively, protein structure prediction tools can be used for protein structure modelling to identify essential amino acids and/or active sites of polypeptides. See, for example, Jumper et al., 2021, “Highly accurate protein structure prediction with AlphaFold”, Nature 596: 583-589.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and regiondirected mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The oral care compositions of the invention may comprise invertase in any effective amount or concentration. In a preferred embodiment, the oral care composition comprises from about 1 ppm invertase to about 500 ppm invertase, preferably from about 1 ppm to about 100 ppm, more preferably from about 5 ppm to about 75 ppm, even more preferably from about 10 ppm to about 60 ppm, most preferably from 10 ppm to 60 ppm.

In preferred embodiment, the oral care composition comprises invertase in an amount of at least 1 ppm, e.g., at least 5 ppm, at least 10 ppm, at least 15 ppm, at least 20 ppm, at least 25 ppm, at least 30 ppm, at least 35 ppm, at least 40 ppm, at least 45 ppm, at least 50 ppm, at least 55 ppm, at least 60 ppm, at least 65 ppm, at least 70 ppm, at least 75 ppm, at least 80 ppm, at least 85 ppm, at least 90 ppm, at least 95 ppm, at least 100 ppm, or more.

In a particularly preferred embodiment, the oral care composition comprises at least 10 ppm invertase. In another particularly preferred embodiment, the oral care composition comprises at least 60 ppm invertase.

The invertase prevents formation of oral biofilm. Preferably, the invertase has improved effect on oral biofilm prevention. In an embodiment, the invertase prevents formation of oral biofilm by at least 5%, e.g., 10%, 15%, 20%, 25%, 30%, 40%, 50%, 60%, 70%, 80%, 90%, or even 100%. For purposes of the present invention, oral biofilm prevention may be determined, e.g., according to Example 4 below.

The invertase reduces the risk of oral biofilm formation. Preferably, the invertase reduces the risk of oral biofilm formation by at least 5%, e.g., 10%, 15%, 20%, 25%, 30%, 40%, 50%, 60%, 70%, 80%, 90%, or even 100%.

The invertase may also remove oral biofilm. Preferably, the invertase has improved effect on oral biofilm removal. In an embodiment, the invertase removes at least 5%, e.g., 10%, 15%, 20%, 25%, 30%, 40%, 50%, 60%, 70%, 80%, 90%, or even 100%, of oral biofilm.

Invertases are highly stabile in formulations and/or formats suitable for oral care, in particular formulations or formats such as toothpastes, mouthwashes, lozenges, mints, gums, candy, etc. The high stability, e.g., on par or improved stability, may be on par or improved physical and/or chemical stability. On par or improved chemical stability, i.e., on par or improved stability in the presence of another agent (e.g., another enzyme, an active ingredient, an excipient, or a solvent) may occur when the invertase and the other agent are co-formulated and/or co-administered, preferably upon co-formulation.

In a preferred embodiment, the invertase has on par or improved thermal stability. In the context of the present invention, the term “on par thermal stability” means that the thermal stability of an invertase in the presence of (or, alternatively stated, co-formulated with) a particular oral care ingredient or component is within +/−5% of the thermal stability of the same invertase alone (i.e., in the absence of said oral care ingredient). In the context of the present invention, the term “improved thermal stability” means that the thermal stability of an invertase in the presence of (or, alternatively stated, co-formulated with) a particular oral care ingredient or component is improved by at least 5%, e.g., at least 10%, at least 15%, at least 20%, at least 25%, at least 30%, at least 40%, at least 50%, at least 60%, at least 70%, at least 80%, at least 90%, at least 100%, or even more, compared to the thermal stability of the same invertase alone (i.e., in the absence of said oral care ingredient). For purposes of the present invention, thermal stability may be determined according to Example 3 below and is defined by the thermal unfolding transition midpoint (Tm).

In one embodiment, the invertase has on par or improved thermal stability in the presence of at least one, e.g., at least two, at least three, at least four, at least five, at least six, at least seven, at least eight, at least nine, at least ten, or all, oral care ingredient(s) selected from the group consisting of benzoate (preferably sodium benzoate), EDTA, ethanol, fluoride (preferably sodium fluoride), glycerol, hydrogen peroxide, mannitol, phosphate (preferably sodium phosphate), SDS, sorbate (preferably potassium sorbate), and sorbitol.

In a preferred embodiment, the invertase has on par or improved thermal stability at pH 4-8, e.g., at pH 4, 5, 6, 7, or 8. Preferably, the invertase has on par or improved thermal stability at pH 5-7, more preferably at pH 5-6, most preferably at pH 5 and/or pH 6.

In one embodiment, the oral care composition comprises benzoate, e.g., sodium benzoate, and the invertase has on par or improved thermal stability in the presence of benzoate, e.g., sodium benzoate. Preferably, the invertase has on par or improved thermal stability in the presence of 0.01-5% benzoate (e.g., sodium benzoate), more preferably 0.05-2.5% benzoate, even more preferably 0.1-1% benzoate, most preferably 0.1-0.5% benzoate. Preferably, the invertase has on par or improved thermal stability in the presence of 1-100 mM benzoate (e.g., sodium benzoate), more preferably 5-50 mM benzoate, most preferably 10-35 mM benzoate.

In one embodiment, the oral care composition comprises EDTA, and the invertase has on par or improved thermal stability in the presence of EDTA. Preferably, the invertase has on par or improved thermal stability in the presence 0.1-10 mM EDTA, more preferably 0.5-5 mM EDTA, most preferably 1 mM EDTA.

In one embodiment, the oral care composition comprises ethanol, and the invertase has on par or improved thermal stability in the presence of ethanol. Preferably, the invertase has on par or improved thermal stability in the presence of 0.1-20% ethanol, more preferably 1-10% ethanol, even more preferably 2.5-7.5% ethanol, most preferably 5% ethanol. Preferably, the invertase has on par or improved thermal stability in the presence of 1-100000 mM ethanol, more preferably 100-10000 mM ethanol, most preferably 1000 mM ethanol.

In one embodiment, the oral care composition comprises fluoride, e.g., sodium fluoride, sodium monofluorophosphate, calcium fluoride, or stannous fluoride, and the invertase has on par or improved thermal stability in the presence of fluoride, e.g., sodium fluoride, sodium monofluorophosphate, calcium fluoride, or stannous fluoride. Preferably, the invertase has on par or improved thermal stability in the presence of 1-5000 ppm fluoride (e.g., sodium fluoride), more preferably 500-2500 ppm fluoride, most preferably 1,000-1500 ppm fluoride. Preferably, the invertase has on par or improved thermal stability in the presence of 1-100 mM fluoride (e.g., sodium fluoride), more preferably 5-75 mM fluoride, even more preferably 10-50 mM fluoride, most preferably 20-40 mM fluoride.

In one embodiment, the oral care composition comprises glycerol, and the invertase has on par or improved thermal stability in the presence of glycerol. Preferably, the invertase has on par or improved thermal stability in the presence of 1-50% glycerol, more preferably 5-40% glycerol, most preferably 10-30% glycerol. Preferably, the invertase has on par or improved thermal stability in the presence of 100-10000 mM glycerol, more preferably 500-5000 mM glycerol, even more preferably 750-4000 mM glycerol, most preferably 1000-3250 mM glycerol.

In one embodiment, the oral care composition comprises peroxide, e.g., hydrogen peroxide, and the invertase has on par or improved thermal stability in the presence of peroxide, e.g., hydrogen peroxide. Preferably, the invertase has on par or improved thermal stability in the presence of 1-1000 mM peroxide, more preferably 50-750 mM peroxide, most preferably 100-500 mM peroxide.

In one embodiment, the oral care composition comprises mannitol, and the invertase has on par or improved thermal stability in the presence of mannitol. Preferably, the invertase has on par or improved thermal stability in the presence of 1-1000 mM mannitol, more preferably 150-750 mM mannitol, most preferably 250-550 mM mannitol.

In one embodiment, the oral care composition comprises phosphate, e.g., sodium phosphate or potassium phosphate, and the invertase has on par or improved thermal stability in the presence of phosphate, e.g., sodium phosphate or potassium phosphate. Preferably, the invertase has on par or improved thermal stability in the presence of 1-50 mM phosphate (e.g., sodium phosphate), more preferably 2.5-25 mM phosphate, even more preferably 5-10 mM phosphate.

In one embodiment, the oral care composition comprises sodium dodecyl sulphate (SDS), and the invertase has on par or improved thermal stability in the presence of SDS. Preferably, the invertase has on par or improved thermal stability in the presence of 10-50 mM SDS, more preferably 15-25 mM SDS, most preferably 17 mM SDS.

In one embodiment, the oral care composition comprises sorbate, e.g., sodium sorbate, potassium sorbate, or calcium sorbate, and the invertase has on par or improved thermal stability in the presence of sorbate, e.g., sodium sorbate, potassium sorbate, or calcium sorbate. Preferably, the invertase has on par or improved thermal stability in the presence of 0.01-5% sorbate (e.g., potassium sorbate), more preferably 0.05-2.5% sorbate, even more preferably 0.1-1% sorbate, most preferably 0.1-0.5% sorbate. Preferably, the invertase has on par or improved thermal stability in the presence of 1-100 mM sorbate (e.g., potassium sorbate), more preferably 5-75 mM sorbate, even more preferably 7.5-50 mM sorbate, most preferably 10-35 mM sorbate.

In one embodiment, the oral care composition comprises sorbitol, and the invertase has on par or improved thermal stability in the presence of sorbitol. Preferably, the invertase has on par or improved thermal stability in the presence of 0.1-70% sorbitol, more preferably 1-60% sorbitol, even more preferably 5-50% sorbitol, most preferably 10-40% sorbitol. Preferably, the invertase has on par or improved thermal stability in the presence of 100-10000 mM sorbitol, more preferably 250-5000 mM sorbitol, even more preferably 500-2500 mM sorbitol, most preferably 550-2200 mM sorbitol.

### Beta-Glucosidases

The beta-glucosidase may be derived from any organism. Preferably, the beta-glucosidase is of microbial origin; most preferably, the beta-glucosidase is of bacterial or fungal origin. In a preferred embodiment, the beta-glucosidase is derived from Aspergillus niger.

In one aspect, the beta-glucosidase is selected from the group consisting of:
(a) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:5;
(b) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:6;
c) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:5;
(d) a polypeptide encoded by a polynucleotide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to the mature polypeptide coding sequence of SEQ ID NO:4 or the cDNA sequence thereof;
(e) a polypeptide derived from SEQ ID NO:5, a mature polypeptide of SEQ ID NO:5, or SEQ ID NO:6 by substitution, deletion, or addition of one or several amino acids;
(f) a polypeptide derived from the polypeptide of (a), (b), (c), (d) or (e) wherein the N- and/or C-terminal end has been extended by the addition of one or more amino acids; and
(g) a fragment of the polypeptide of (a), (b), (c), (d), or (e); wherein the polypeptide has beta-glucosidase activity.

In a preferred embodiment, the beta-glucosidase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:5 or a mature polypeptide of SEQ ID NO:5. A preferred mature polypeptide of SEQ ID NO:5 corresponds to amino acid residues 20 to 860 of SEQ ID NO:5.

In a preferred embodiment, the beta-glucosidase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:6.

In a preferred embodiment, the beta-glucosidase comprises, consists essentially of, or consists of SEQ ID NO:5 or a mature polypeptide of SEQ ID NO:5. A preferred mature polypeptide of SEQ ID NO:5 corresponds to amino acid residues 20 to 860 of SEQ ID NO:5.

In a preferred embodiment, the beta-glucosidase comprises, consists essentially of, or consists of SEQ ID NO:6 or a fragment thereof.

The beta-glucosidase may have an N-terminal and/or C-terminal extension of one or more amino acids, e.g., 1-5 amino acids.

In another aspect, the beta-glucosidase is derived from SEQ ID NO:5 by substitution, deletion, or addition of one or several amino acids. In another aspect, the polypeptide is derived from a mature polypeptide of SEQ ID NO:5 by substitution, deletion, or addition of one or several amino acids. In another aspect, the polypeptide is derived from SEQ ID NO:6 by substitution, deletion, or addition of one or more amino acids.

In some embodiments, the beta-glucosidase is a variant of parent beta-glucosidase, preferably SEQ ID NO:6, comprising a substitution, deletion, and/or insertion at one or more positions. In one aspect, the beta-glucosidase is a variant of SEQ ID NO:6 and the number of amino acid substitutions, deletions and/or insertions introduced into the polypeptide of SEQ ID NO:6 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant molecules are tested for beta-glucosidase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide, and/or be inferred from sequence homology and conserved catalytic machinery with a related polypeptide or within a polypeptide or protein family with polypeptides/proteins descending from a common ancestor, typically having similar three-dimensional structures, functions, and significant sequence similarity. Additionally, or alternatively, protein structure prediction tools can be used for protein structure modelling to identify essential amino acids and/or active sites of polypeptides. See, for example, Jumper et al., 2021, “Highly accurate protein structure prediction with AlphaFold”, Nature 596: 583-589.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and regiondirected mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The oral care compositions of the invention may comprise beta-glucosidase in any effective amount or concentration. In a preferred embodiment, the oral care composition comprises from about 1 ppm beta-glucosidase to about 500 ppm beta-glucosidase, preferably from about 1 ppm to about 100 ppm, more preferably from about 5 ppm to about 75 ppm, even more preferably from about 10 ppm to about 60 ppm, most preferably from 10 ppm to 60 ppm.

In preferred embodiment, the oral care composition comprises beta-glucosidase in an amount of at least 1 ppm, e.g., at least 5 ppm, at least 10 ppm, at least 15 ppm, at least 20 ppm, at least 25 ppm, at least 30 ppm, at least 35 ppm, at least 40 ppm, at least 45 ppm, at least 50 ppm, at least 55 ppm, at least 60 ppm, at least 65 ppm, at least 70 ppm, at least 75 ppm, at least 80 ppm, at least 85 ppm, at least 90 ppm, at least 95 ppm, at least 100 ppm, or more.

In a particularly preferred embodiment, the oral care composition comprises at least 10 ppm beta-glucosidase. In another particularly preferred embodiment, the oral care composition comprises at least 60 ppm beta-glucosidase.

The beta-glucosidase prevents formation of oral biofilm. Preferably, the beta-glucosidase has improved effect on oral biofilm prevention. In an embodiment, the beta-glucosidase prevents formation of oral biofilm by at least 5%, e.g., 10%, 15%, 20%, 25%, 30%, 40%, 50%, 60%, 70%, 80%, 90%, or even 100%. For purposes of the present invention, oral biofilm prevention may be determined, e.g., according to Example 4 below.

The beta-glucosidase reduces the risk of oral biofilm formation. Preferably, the beta-glucosidase reduces the risk of oral biofilm formation by at least 5%, e.g., 10%, 15%, 20%, 25%, 30%, 40%, 50%, 60%, 70%, 80%, 90%, or even 100%.

The beta-glucosidase may also remove oral biofilm. Preferably, the beta-glucosidase has improved effect on oral biofilm removal. In an embodiment, the beta-glucosidase removes at least 5%, e.g., 10%, 15%, 20%, 25%, 30%, 40%, 50%, 60%, 70%, 80%, 90%, or even 100%, of oral biofilm.

Beta-glucosidases are highly stabile in formulations and/or formats suitable for oral care, in particular formulations or formats such as toothpastes, mouthwashes, lozenges, mints, gums, candy, etc. The high stability, e.g., on par or improved stability, may be on par or improved physical and/or chemical stability. On par or improved chemical stability, i.e., on par or improved stability in the presence of another agent (e.g., another enzyme, an active ingredient, an excipient, or a solvent) may occur when the beta-glucosidase and the other agent are co-formulated and/or co-administered, preferably upon co-formulation.

In a preferred embodiment, the beta-glucosidase has on par or improved thermal stability. In the context of the present invention, the term “on par thermal stability” means that the thermal stability of a beta-glucosidase in the presence of (or, alternatively stated, co-formulated with) a particular oral care ingredient or component is within +/−5% of the thermal stability of the same beta-glucosidase alone (i.e., in the absence of said oral care ingredient). In the context of the present invention, the term “improved thermal stability” means that the thermal stability of an beta-glucosidase in the presence of (or, alternatively stated, co-formulated with) a particular oral care ingredient or component is improved by at least 5%, e.g., at least 10%, at least 15%, at least 20%, at least 25%, at least 30%, at least 40%, at least 50%, at least 60%, at least 70%, at least 80%, at least 90%, at least 100%, or even more, compared to the thermal stability of the same beta-glucosidase alone (i.e., in the absence of said oral care ingredient). For purposes of the present invention, thermal stability may be determined according to Example 3 below and is defined by the thermal unfolding transition midpoint (Tm).

In one embodiment, the beta-glucosidase has on par or improved thermal stability in the presence of at least one, e.g., at least two, at least three, at least four, at least five, at least six, at least seven, at least eight, at least nine, at least ten, or all, oral care ingredient(s) selected from the group consisting of benzoate (preferably sodium benzoate), EDTA, ethanol, fluoride (preferably sodium fluoride), glycerol, hydrogen peroxide, mannitol, phosphate (preferably sodium phosphate), SDS, sorbate (preferably potassium sorbate), and sorbitol.

In a preferred embodiment, the beta-glucosidase has on par or improved thermal stability at pH 4-8, e.g., at pH 4, 5, 6, 7, or 8. Preferably, the beta-glucosidase has on par or improved thermal stability at pH 5-7, more preferably at pH 5-6, most preferably at pH 5 and/or pH 6.

In one embodiment, the oral care composition comprises benzoate, e.g., sodium benzoate, and the beta-glucosidase has on par or improved thermal stability in the presence of benzoate, e.g., sodium benzoate. Preferably, the beta-glucosidase has on par or improved thermal stability in the presence of 0.01-5% benzoate (e.g., sodium benzoate), more preferably 0.05-2.5% benzoate, even more preferably 0.1-1% benzoate, most preferably 0.1-0.5% benzoate. Preferably, the beta-glucosidase has on par or improved thermal stability in the presence of 1-100 mM benzoate (e.g., sodium benzoate), more preferably 5-50 mM benzoate, most preferably 10-35 mM benzoate.

In one embodiment, the oral care composition comprises EDTA, and the beta-glucosidase has on par or improved thermal stability in the presence of EDTA. Preferably, the beta-glucosidase has on par or improved thermal stability in the presence 0.1-10 mM EDTA, more preferably 0.5-5 mM EDTA, most preferably 1 mM EDTA.

In one embodiment, the oral care composition comprises ethanol, and the beta-glucosidase has on par or improved thermal stability in the presence of ethanol. Preferably, the beta-glucosidase has on par or improved thermal stability in the presence of 0.1-20% ethanol, more preferably 1-10% ethanol, even more preferably 2.5-7.5% ethanol, most preferably 5% ethanol. Preferably, the beta-glucosidase has on par or improved thermal stability in the presence of 1-100000 mM ethanol, more preferably 100-10000 mM ethanol, most preferably 1000 mM ethanol.

In one embodiment, the oral care composition comprises fluoride, e.g., sodium fluoride, sodium monofluorophosphate, calcium fluoride, or stannous fluoride, and the beta-glucosidase has on par or improved thermal stability in the presence of fluoride, e.g., sodium fluoride, sodium monofluorophosphate, calcium fluoride, or stannous fluoride. Preferably, the beta-glucosidase has on par or improved thermal stability in the presence of 1-5000 ppm fluoride (e.g., sodium fluoride), more preferably 500-2500 ppm fluoride, most preferably 1,000-1500 ppm fluoride. Preferably, the beta-glucosidase has on par or improved thermal stability in the presence of 1-100 mM fluoride (e.g., sodium fluoride), more preferably 5-75 mM fluoride, even more preferably 10-50 mM fluoride, most preferably 20-40 mM fluoride.

In one embodiment, the oral care composition comprises glycerol, and the beta-glucosidase has on par or improved thermal stability in the presence of glycerol. Preferably, the beta-glucosidase has on par or improved thermal stability in the presence of 1-50% glycerol, more preferably 5-40% glycerol, most preferably 10-30% glycerol. Preferably, the beta-glucosidase has on par or improved thermal stability in the presence of 100-10000 mM glycerol, more preferably 500-5000 mM glycerol, even more preferably 750-4000 mM glycerol, most preferably 1000-3250 mM glycerol.

In one embodiment, the oral care composition comprises peroxide, e.g., hydrogen peroxide, and the beta-glucosidase has on par or improved thermal stability in the presence of peroxide, e.g., hydrogen peroxide. Preferably, the beta-glucosidase has on par or improved thermal stability in the presence of 1-1000 mM peroxide, more preferably 50-750 mM peroxide, most preferably 100-500 mM peroxide.

In one embodiment, the oral care composition comprises mannitol, and the beta-glucosidase has on par or improved thermal stability in the presence of mannitol. Preferably, the beta-glucosidase has on par or improved thermal stability in the presence of 1-1000 mM mannitol, more preferably 150-750 mM mannitol, most preferably 250-550 mM mannitol.

In one embodiment, the oral care composition comprises phosphate, e.g., sodium phosphate or potassium phosphate, and the beta-glucosidase has on par or improved thermal stability in the presence of phosphate, e.g., sodium phosphate or potassium phosphate. Preferably, the beta-glucosidase has on par or improved thermal stability in the presence of 1-50 mM phosphate (e.g., sodium phosphate), more preferably 2.5-25 mM phosphate, even more preferably 5-10 mM phosphate.

In one embodiment, the oral care composition comprises sodium dodecyl sulphate (SDS), and the beta-glucosidase has on par or improved thermal stability in the presence of SDS. Preferably, the beta-glucosidase has on par or improved thermal stability in the presence of 1-1000 mM SDS, more preferably 10-500 mM SDS, more preferably 15-150 mM SDS, most preferably 17-170 mM SDS.

In one embodiment, the oral care composition comprises sorbate, e.g., sodium sorbate, potassium sorbate, or calcium sorbate, and the beta-glucosidase has on par or improved thermal stability in the presence of sorbate, e.g., sodium sorbate, potassium sorbate, or calcium sorbate.

Preferably, the beta-glucosidase has on par or improved thermal stability in the presence of 0.01-5% sorbate (e.g., potassium sorbate), more preferably 0.05-2.5% sorbate, even more preferably 0.1-1% sorbate, most preferably 0.1-0.5% sorbate. Preferably, the beta-glucosidase has on par or improved thermal stability in the presence of 1-100 mM sorbate (e.g., potassium sorbate), more preferably 5-75 mM sorbate, even more preferably 7.5-50 mM sorbate, most preferably 10-35 mM sorbate.

In one embodiment, the oral care composition comprises sorbitol, and the beta-glucosidase has on par or improved thermal stability in the presence of sorbitol. Preferably, the beta-glucosidase has on par or improved thermal stability in the presence of 0.1-70% sorbitol, more preferably 1-60% sorbitol, even more preferably 5-50% sorbitol, most preferably 10-40% sorbitol. Preferably, the beta-glucosidase has on par or improved thermal stability in the presence of 100-10000 mM sorbitol, more preferably 250-5000 mM sorbitol, even more preferably 500-2500 mM sorbitol, most preferably 550-2200 mM sorbitol.

### Glucoamylases

The glucoamylase may be derived from any organism. Preferably, the glucoamylase is of microbial origin; most preferably, the glucoamylase is of bacterial or fungal origin. In a preferred embodiment, the glucoamylase is derived from Aspergillus niger.

In one aspect, the glucoamylase is selected from the group consisting of:
(a) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:8;
(b) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:9;
c) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:8;
(d) a polypeptide encoded by a polynucleotide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to the mature polypeptide coding sequence of SEQ ID NO:7 or the cDNA sequence thereof;
(e) a polypeptide derived from SEQ ID NO:8, a mature polypeptide of SEQ ID NO:8, or SEQ ID NO:9 by substitution, deletion, or addition of one or several amino acids;
(f) a polypeptide derived from the polypeptide of (a), (b), (c), (d) or (e) wherein the N- and/or C-terminal end has been extended by the addition of one or more amino acids; and
(g) a fragment of the polypeptide of (a), (b), (c), (d), or (e); wherein the polypeptide has glucoamylase activity.

In a preferred embodiment, the glucoamylase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:8 or a mature polypeptide of SEQ ID NO:8. A preferred mature polypeptide of SEQ ID NO:8 corresponds to amino acid residues 19 to 640 of SEQ ID NO:8.

In a preferred embodiment, the glucoamylase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:9.

In a preferred embodiment, the glucoamylase comprises, consists essentially of, or consists of SEQ ID NO:8 or a mature polypeptide of SEQ ID NO:8. A preferred mature polypeptide of SEQ ID NO:8 corresponds to amino acid residues 19 to 640 of SEQ ID NO:8.

In a preferred embodiment, the glucoamylase comprises, consists essentially of, or consists of SEQ ID NO:9 or a fragment thereof.

The glucoamylase may have an N-terminal and/or C-terminal extension of one or more amino acids, e.g., 1-5 amino acids.

In another aspect, the glucoamylase is derived from SEQ ID NO:8 by substitution, deletion, or addition of one or several amino acids. In another aspect, the polypeptide is derived from a mature polypeptide of SEQ ID NO:8 by substitution, deletion, or addition of one or several amino acids. In another aspect, the polypeptide is derived from SEQ ID NO:9 by substitution, deletion, or addition of one or more amino acids.

In some embodiments, the glucoamylase is a variant of parent glucoamylase, preferably SEQ ID NO:9, comprising a substitution, deletion, and/or insertion at one or more positions. In one aspect, the glucoamylase is a variant of SEQ ID NO:9 and the number of amino acid substitutions, deletions and/or insertions introduced into the polypeptide of SEQ ID NO:9 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant molecules are tested for glucoamylase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide, and/or be inferred from sequence homology and conserved catalytic machinery with a related polypeptide or within a polypeptide or protein family with polypeptides/proteins descending from a common ancestor, typically having similar three-dimensional structures, functions, and significant sequence similarity. Additionally, or alternatively, protein structure prediction tools can be used for protein structure modelling to identify essential amino acids and/or active sites of polypeptides. See, for example, Jumper et al., 2021, “Highly accurate protein structure prediction with AlphaFold”, Nature 596: 583-589.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The oral care compositions of the invention may comprise glucoamylase in any effective amount or concentration. In a preferred embodiment, the oral care composition comprises from about 1 ppm glucoamylase to about 500 ppm glucoamylase, preferably from about 1 ppm to about 100 ppm, more preferably from about 5 ppm to about 75 ppm, even more preferably from about 10 ppm to about 60 ppm, most preferably from 10 ppm to 60 ppm.

In preferred embodiment, the oral care composition comprises glucoamylase in an amount of at least 1 ppm, e.g., at least 5 ppm, at least 10 ppm, at least 15 ppm, at least 20 ppm, at least 25 ppm, at least 30 ppm, at least 35 ppm, at least 40 ppm, at least 45 ppm, at least 50 ppm, at least 55 ppm, at least 60 ppm, at least 65 ppm, at least 70 ppm, at least 75 ppm, at least 80 ppm, at least 85 ppm, at least 90 ppm, at least 95 ppm, at least 100 ppm, or more.

In a particularly preferred embodiment, the oral care composition comprises at least 10 ppm glucoamylase. In another particularly preferred embodiment, the oral care composition comprises at least 60 ppm glucoamylase.

The glucoamylase prevents formation of oral biofilm. Preferably, the glucoamylase has improved effect on oral biofilm prevention. In an embodiment, the glucoamylase prevents formation of oral biofilm by at least 5%, e.g., 10%, 15%, 20%, 25%, 30%, 40%, 50%, 60%, 70%, 80%, 90%, or even 100%. For purposes of the present invention, oral biofilm prevention may be determined, e.g., according to Example 4 below.

The glucoamylase reduces the risk of oral biofilm formation. Preferably, the glucoamylase reduces the risk of oral biofilm formation by at least 5%, e.g., 10%, 15%, 20%, 25%, 30%, 40%, 50%, 60%, 70%, 80%, 90%, or even 100%.

The glucoamylase may also remove oral biofilm. Preferably, the glucoamylase has improved effect on oral biofilm removal. In an embodiment, the glucoamylase removes at least 5%, e.g., 10%, 15%, 20%, 25%, 30%, 40%, 50%, 60%, 70%, 80%, 90%, or even 100%, of oral biofilm.

Glucoamylases are highly stabile in formulations and/or formats suitable for oral care, in particular formulations or formats such as toothpastes, mouthwashes, lozenges, mints, gums, candy, etc. The high stability, e.g., on par or improved stability, may be on par or improved physical and/or chemical stability. On par or improved chemical stability, i.e., on par or improved stability in the presence of another agent (e.g., another enzyme, an active ingredient, an excipient, or a solvent) may occur when the glucoamylase and the other agent are co-formulated and/or coadministered, preferably upon co-formulation.

In a preferred embodiment, the glucoamylase has on par or improved thermal stability. In the context of the present invention, the term “on par thermal stability” means that the thermal stability of a glucoamylase in the presence of (or, alternatively stated, co-formulated with) a particular oral care ingredient or component is within +/−5% of the thermal stability of the same glucoamylase alone (i.e., in the absence of said oral care ingredient). In the context of the present invention, the term “improved thermal stability” means that the thermal stability of an glucoamylase in the presence of (or, alternatively stated, co-formulated with) a particular oral care ingredient or component is improved by at least 5%, e.g., at least 10%, at least 15%, at least 20%, at least 25%, at least 30%, at least 40%, at least 50%, at least 60%, at least 70%, at least 80%, at least 90%, at least 100%, or even more, compared to the thermal stability of the same glucoamylase alone (i.e., in the absence of said oral care ingredient). For purposes of the present invention, thermal stability may be determined according to Example 3 below and is defined by the thermal unfolding transition midpoint (Tm).

In one embodiment, the glucoamylase has on par or improved thermal stability in the presence of at least one, e.g., at least two, at least three, at least four, at least five, at least six, at least seven, at least eight, at least nine, at least ten, or all, oral care ingredient(s) selected from the group consisting of benzoate (preferably sodium benzoate), EDTA, ethanol, fluoride (preferably sodium fluoride), glycerol, hydrogen peroxide, mannitol, phosphate (preferably sodium phosphate), SDS, sorbate (preferably potassium sorbate), and sorbitol.

In a preferred embodiment, the glucoamylase has on par or improved thermal stability at pH 4-8, e.g., at pH 4, 5, 6, 7, or 8. Preferably, the glucoamylase has on par or improved thermal stability at pH 5-7, more preferably at pH 5-6, most preferably at pH 5 and/or pH 6.

In one embodiment, the oral care composition comprises benzoate, e.g., sodium benzoate, and the glucoamylase has on par or improved thermal stability in the presence of benzoate, e.g., sodium benzoate. Preferably, the glucoamylase has on par or improved thermal stability in the presence of 0.01-5% benzoate (e.g., sodium benzoate), more preferably 0.05-2.5% benzoate, even more preferably 0.1-1% benzoate, most preferably 0.1-0.5% benzoate. Preferably, the glucoamylase has on par or improved thermal stability in the presence of 1-100 mM benzoate (e.g., sodium benzoate), more preferably 5-50 mM benzoate, most preferably 10-35 mM benzoate.

In one embodiment, the oral care composition comprises EDTA, and the glucoamylase has on par or improved thermal stability in the presence of EDTA. Preferably, the glucoamylase has on par or improved thermal stability in the presence 0.1-10 mM EDTA, more preferably 0.5-5 mM EDTA, most preferably 1 mM EDTA.

In one embodiment, the oral care composition comprises ethanol, and the glucoamylase has on par or improved thermal stability in the presence of ethanol. Preferably, the glucoamylase has on par or improved thermal stability in the presence of 0.1-20% ethanol, more preferably 1-10% ethanol, even more preferably 2.5-7.5% ethanol, most preferably 5% ethanol. Preferably, the glucoamylase has on par or improved thermal stability in the presence of 1-100000 mM ethanol, more preferably 100-10000 mM ethanol, most preferably 1000 mM ethanol.

In one embodiment, the oral care composition comprises fluoride, e.g., sodium fluoride, sodium monofluorophosphate, calcium fluoride, or stannous fluoride, and the glucoamylase has on par or improved thermal stability in the presence of fluoride, e.g., sodium fluoride, sodium monofluorophosphate, calcium fluoride, or stannous fluoride. Preferably, the glucoamylase has on par or improved thermal stability in the presence of 1-5000 ppm fluoride (e.g., sodium fluoride), more preferably 500-2500 ppm fluoride, most preferably 1,000-1500 ppm fluoride. Preferably, the glucoamylase has on par or improved thermal stability in the presence of 1-100 mM fluoride (e.g., sodium fluoride), more preferably 5-75 mM fluoride, even more preferably 10-50 mM fluoride, most preferably 20-40 mM fluoride.

In one embodiment, the oral care composition comprises glycerol, and the glucoamylase has on par or improved thermal stability in the presence of glycerol. Preferably, the glucoamylase has on par or improved thermal stability in the presence of 1-50% glycerol, more preferably 5-40% glycerol, most preferably 10-30% glycerol. Preferably, the glucoamylase has on par or improved thermal stability in the presence of 100-10000 mM glycerol, more preferably 500-5000 mM glycerol, even more preferably 750-4000 mM glycerol, most preferably 1000-3250 mM glycerol.

In one embodiment, the oral care composition comprises peroxide, e.g., hydrogen peroxide, and the glucoamylase has on par or improved thermal stability in the presence of peroxide, e.g., hydrogen peroxide. Preferably, the glucoamylase has on par or improved thermal stability in the presence of 1-1000 mM peroxide, more preferably 50-750 mM peroxide, most preferably 100-500 mM peroxide.

In one embodiment, the oral care composition comprises mannitol, and the glucoamylase has on par or improved thermal stability in the presence of mannitol. Preferably, the glucoamylase has on par or improved thermal stability in the presence of 1-1000 mM mannitol, more preferably 150-750 mM mannitol, most preferably 250-550 mM mannitol.

In one embodiment, the oral care composition comprises phosphate, e.g., sodium phosphate or potassium phosphate, and the glucoamylase has on par or improved thermal stability in the presence of phosphate, e.g., sodium phosphate or potassium phosphate. Preferably, the glucoamylase has on par or improved thermal stability in the presence of 1-50 mM phosphate (e.g., sodium phosphate), more preferably 2.5-25 mM phosphate, even more preferably 5-10 mM phosphate.

In one embodiment, the oral care composition comprises sodium dodecyl sulphate (SDS), and the glucoamylase has on par or improved thermal stability in the presence of SDS. Preferably, the glucoamylase has on par or improved thermal stability in the presence of 1-1000 mM SDS, more preferably 10-500 mM SDS, more preferably 15-150 mM SDS, most preferably 17-170 mM SDS.

In one embodiment, the oral care composition comprises sorbate, e.g., sodium sorbate, potassium sorbate, or calcium sorbate, and the glucoamylase has on par or improved thermal stability in the presence of sorbate, e.g., sodium sorbate, potassium sorbate, or calcium sorbate. Preferably, the glucoamylase has on par or improved thermal stability in the presence of 0.01-5% sorbate (e.g., potassium sorbate), more preferably 0.05-2.5% sorbate, even more preferably 0.1-1% sorbate, most preferably 0.1-0.5% sorbate. Preferably, the glucoamylase has on par or improved thermal stability in the presence of 1-100 mM sorbate (e.g., potassium sorbate), more preferably 5-75 mM sorbate, even more preferably 7.5-50 mM sorbate, most preferably 10-35 mM sorbate.

In one embodiment, the oral care composition comprises sorbitol, and the glucoamylase has on par or improved thermal stability in the presence of sorbitol. Preferably, the glucoamylase has on par or improved thermal stability in the presence of 0.1-70% sorbitol, more preferably 1-60% sorbitol, even more preferably 5-50% sorbitol, most preferably 10-40% sorbitol. Preferably, the glucoamylase has on par or improved thermal stability in the presence of 100-10000 mM sorbitol, more preferably 250-5000 mM sorbitol, even more preferably 500-2500 mM sorbitol, most preferably 550-2200 mM sorbitol.

### Alpha-Amylases

The alpha-amylase may be derived from any organism. Preferably, the alpha-amylase is of microbial origin; most preferably, the alpha-amylase is of bacterial or fungal origin. In a preferred embodiment, the alpha-amylase is derived from Bacillus amyloliquefaciens.

In one aspect, the alpha-amylase is selected from the group consisting of:
(a) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:50;
(b) a polypeptide encoded by a polynucleotide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to the mature polypeptide coding sequence of SEQ ID NO:49 or the cDNA sequence thereof;
(c) a polypeptide derived from SEQ ID NO:50 by substitution, deletion, or addition of one or several amino acids;
(f) a polypeptide derived from the polypeptide of (a), (b), or (c), wherein the N- and/or C-terminal end has been extended by the addition of one or more amino acids; and
(g) a fragment of the polypeptide of (a), (b), or (c), wherein the polypeptide has alpha-amylase activity.

In a preferred embodiment, the alpha-amylase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:50.

In a preferred embodiment, the alpha-amylase comprises, consists essentially of, or consists of SEQ ID NO:50 or a fragment thereof.

The alpha-amylase may have an N-terminal and/or C-terminal extension of one or more amino acids, e.g., 1-5 amino acids.

In another aspect, the alpha-amylase is derived from SEQ ID NO:50 by substitution, deletion, or addition of one or several amino acids. In another aspect, the polypeptide is derived from a mature polypeptide of SEQ ID NO:50 by substitution, deletion, or addition of one or several amino acids.

In some embodiments, the alpha-amylase is a variant of parent alpha-amylase, preferably SEQ ID NO:50, comprising a substitution, deletion, and/or insertion at one or more positions. In one aspect, the alpha-amylase is a variant of SEQ ID NO:50 and the number of amino acid substitutions, deletions and/or insertions introduced into the polypeptide of SEQ ID NO:50 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant molecules are tested for alpha-amylase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide, and/or be inferred from sequence homology and conserved catalytic machinery with a related polypeptide or within a polypeptide or protein family with polypeptides/proteins descending from a common ancestor, typically having similar three-dimensional structures, functions, and significant sequence similarity. Additionally, or alternatively, protein structure prediction tools can be used for protein structure modelling to identify essential amino acids and/or active sites of polypeptides. See, for example, Jumper et al., 2021, “Highly accurate protein structure prediction with AlphaFold”, Nature 596: 583-589.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and regiondirected mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The oral care compositions of the invention may comprise alpha-amylase in any effective amount or concentration. In a preferred embodiment, the oral care composition comprises from about 1 ppm alpha-amylase to about 500 ppm alpha-amylase, preferably from about 1 ppm to about 100 ppm, more preferably from about 5 ppm to about 75 ppm, even more preferably from about 10 ppm to about 60 ppm, most preferably from 10 ppm to 60 ppm.

In preferred embodiment, the oral care composition comprises alpha-amylase in an amount of at least 1 ppm, e.g., at least 5 ppm, at least 10 ppm, at least 15 ppm, at least 20 ppm, at least 25 ppm, at least 30 ppm, at least 35 ppm, at least 40 ppm, at least 45 ppm, at least 50 ppm, at least 55 ppm, at least 60 ppm, at least 65 ppm, at least 70 ppm, at least 75 ppm, at least 80 ppm, at least 85 ppm, at least 90 ppm, at least 95 ppm, at least 100 ppm, or more.

In a particularly preferred embodiment, the oral care composition comprises at least 10 ppm alpha-amylase. In another particularly preferred embodiment, the oral care composition comprises at least 60 ppm alpha-amylase.

The alpha-amylase prevents formation of oral biofilm. Preferably, the alpha-amylase has improved effect on oral biofilm prevention. In an embodiment, the alpha-amylase prevents formation of oral biofilm by at least 5%, e.g., 10%, 15%, 20%, 25%, 30%, 40%, 50%, 60%, 70%, 80%, 90%, or even 100%. For purposes of the present invention, oral biofilm prevention may be determined, e.g., according to Example 4 below.

The alpha-amylase reduces the risk of oral biofilm formation. Preferably, the alpha-amylase reduces the risk of oral biofilm formation by at least 5%, e.g., 10%, 15%, 20%, 25%, 30%, 40%, 50%, 60%, 70%, 80%, 90%, or even 100%.

The alpha-amylase may also remove oral biofilm. Preferably, the alpha-amylase has improved effect on oral biofilm removal. In an embodiment, the alpha-amylase removes at least 5%, e.g., 10%, 15%, 20%, 25%, 30%, 40%, 50%, 60%, 70%, 80%, 90%, or even 100%, of oral biofilm.

Alpha-amylases are highly stabile in formulations and/or formats suitable for oral care, in particular formulations or formats such as toothpastes, mouthwashes, lozenges, mints, gums, candy, etc. The high stability, e.g., on par or improved stability, may be on par or improved physical and/or chemical stability. On par or improved chemical stability, i.e., on par or improved stability in the presence of another agent (e.g., another enzyme, an active ingredient, an excipient, or a solvent) may occur when the alpha-amylase and the other agent are co-formulated and/or co-administered, preferably upon co-formulation.

In a preferred embodiment, the alpha-amylase has on par or improved thermal stability. In the context of the present invention, the term “on par thermal stability” means that the thermal stability of an alpha-amylase in the presence of (or, alternatively stated, co-formulated with) a particular oral care ingredient or component is within +/−5% of the thermal stability of the same alpha-amylase alone (i.e., in the absence of said oral care ingredient). In the context of the present invention, the term “improved thermal stability” means that the thermal stability of an alphaamylase in the presence of (or, alternatively stated, co-formulated with) a particular oral care ingredient or component is improved by at least 5%, e.g., at least 10%, at least 15%, at least 20%, at least 25%, at least 30%, at least 40%, at least 50%, at least 60%, at least 70%, at least 80%, at least 90%, at least 100%, or even more, compared to the thermal stability of the same alpha-amylase alone (i.e., in the absence of said oral care ingredient). For purposes of the present invention, thermal stability may be determined according to Example 3 below and is defined by the thermal unfolding transition midpoint (Tm).

In one embodiment, the alpha-amylase has on par or improved thermal stability in the presence of at least one, e.g., at least two, at least three, at least four, at least five, at least six, at least seven, at least eight, at least nine, at least ten, or all, oral care ingredient(s) selected from the group consisting of benzoate (preferably sodium benzoate), EDTA, ethanol, fluoride (preferably sodium fluoride), glycerol, hydrogen peroxide, mannitol, phosphate (preferably sodium phosphate), SDS, sorbate (preferably potassium sorbate), and sorbitol.

In a preferred embodiment, the alpha-amylase has on par or improved thermal stability at pH 4-8, e.g., at pH 4, 5, 6, 7, or 8. Preferably, the alpha-amylase has on par or improved thermal stability at pH 5-7, more preferably at pH 5-6, most preferably at pH 5 and/or pH 6.

In one embodiment, the oral care composition comprises benzoate, e.g., sodium benzoate, and the alpha-amylase has on par or improved thermal stability in the presence of benzoate, e.g., sodium benzoate. Preferably, the alpha-amylase has on par or improved thermal stability in the presence of 0.01-5% benzoate (e.g., sodium benzoate), more preferably 0.05-2.5% benzoate, even more preferably 0.1-1% benzoate, most preferably 0.1-0.5% benzoate. Preferably, the alpha-amylase has on par or improved thermal stability in the presence of 1-100 mM benzoate (e.g., sodium benzoate), more preferably 5-50 mM benzoate, most preferably 10-35 mM benzoate.

In one embodiment, the oral care composition comprises EDTA, and the alpha-amylase has on par or improved thermal stability in the presence of EDTA. Preferably, the alpha-amylase has on par or improved thermal stability in the presence 0.1-10 mM EDTA, more preferably 0.5-5 mM EDTA, most preferably 1 mM EDTA.

In one embodiment, the oral care composition comprises ethanol, and the alpha-amylase has on par or improved thermal stability in the presence of ethanol. Preferably, the alpha-amylase has on par or improved thermal stability in the presence of 0.1-20% ethanol, more preferably 1-10% ethanol, even more preferably 2.5-7.5% ethanol, most preferably 5% ethanol. Preferably, the alpha-amylase has on par or improved thermal stability in the presence of 1-100000 mM ethanol, more preferably 100-10000 mM ethanol, most preferably 1000 mM ethanol.

In one embodiment, the oral care composition comprises fluoride, e.g., sodium fluoride, sodium monofluorophosphate, calcium fluoride, or stannous fluoride, and the alpha-amylase has on par or improved thermal stability in the presence of fluoride, e.g., sodium fluoride, sodium monofluorophosphate, calcium fluoride, or stannous fluoride. Preferably, the alpha-amylase has on par or improved thermal stability in the presence of 1-5000 ppm fluoride (e.g., sodium fluoride), more preferably 500-2500 ppm fluoride, most preferably 1,000-1500 ppm fluoride. Preferably, the alpha-amylase has on par or improved thermal stability in the presence of 1-100 mM fluoride (e.g., sodium fluoride), more preferably 5-75 mM fluoride, even more preferably 10-50 mM fluoride, most preferably 20-40 mM fluoride.

In one embodiment, the oral care composition comprises glycerol, and the alpha-amylase has on par or improved thermal stability in the presence of glycerol. Preferably, the alpha-amylase has on par or improved thermal stability in the presence of 1-50% glycerol, more preferably 5-40% glycerol, most preferably 10-30% glycerol. Preferably, the alpha-amylase has on par or improved thermal stability in the presence of 100-10000 mM glycerol, more preferably 500-5000 mM glycerol, even more preferably 750-4000 mM glycerol, most preferably 1000-3250 mM glycerol.

In one embodiment, the oral care composition comprises peroxide, e.g., hydrogen peroxide, and the alpha-amylase has on par or improved thermal stability in the presence of peroxide, e.g., hydrogen peroxide. Preferably, the alpha-amylase has on par or improved thermal stability in the presence of 1-1000 mM peroxide, more preferably 50-750 mM peroxide, most preferably 100-500 mM peroxide.

In one embodiment, the oral care composition comprises mannitol, and the alpha-amylase has on par or improved thermal stability in the presence of mannitol. Preferably, the alpha-amylase has on par or improved thermal stability in the presence of 1-1000 mM mannitol, more preferably 150-750 mM mannitol, most preferably 250-550 mM mannitol.

In one embodiment, the oral care composition comprises phosphate, e.g., sodium phosphate or potassium phosphate, and the alpha-amylase has on par or improved thermal stability in the presence of phosphate, e.g., sodium phosphate or potassium phosphate. Preferably, the alpha-amylase has on par or improved thermal stability in the presence of 1-50 mM phosphate (e.g., sodium phosphate), more preferably 2.5-25 mM phosphate, even more preferably 5-10 mM phosphate.

In one embodiment, the oral care composition comprises sodium dodecyl sulphate (SDS), and the alpha-amylase has on par or improved thermal stability in the presence of SDS. Preferably, the alpha-amylase has on par or improved thermal stability in the presence of 10-50 mM SDS, more preferably 15-25 mM SDS, most preferably 17 mM SDS.

In one embodiment, the oral care composition comprises sorbate, e.g., sodium sorbate, potassium sorbate, or calcium sorbate, and the alpha-amylase has on par or improved thermal stability in the presence of sorbate, e.g., sodium sorbate, potassium sorbate, or calcium sorbate.

Preferably, the alpha-amylase has on par or improved thermal stability in the presence of 0.01-5% sorbate (e.g., potassium sorbate), more preferably 0.05-2.5% sorbate, even more preferably 0.1-1% sorbate, most preferably 0.1-0.5% sorbate. Preferably, the alpha-amylase has on par or improved thermal stability in the presence of 1-100 mM sorbate (e.g., potassium sorbate), more preferably 5-75 mM sorbate, even more preferably 7.5-50 mM sorbate, most preferably 10-35 mM sorbate.

In one embodiment, the oral care composition comprises sorbitol, and the alpha-amylase has on par or improved thermal stability in the presence of sorbitol. Preferably, the alpha-amylase has on par or improved thermal stability in the presence of 0.1-70% sorbitol, more preferably 1-60% sorbitol, even more preferably 5-50% sorbitol, most preferably 10-40% sorbitol. Preferably, the alpha-amylase has on par or improved thermal stability in the presence of 100-10000 mM sorbitol, more preferably 250-5000 mM sorbitol, even more preferably 500-2500 mM sorbitol, most preferably 550-2200 mM sorbitol.

### Oral Care Ingredients and Formats

The oral care compositions of the invention comprise an invertase, a beta-glucosidase, a glucoamylase, and at least one oral care ingredient. In a preferred embodiment, the oral care composition further comprises an alpha-amylase.

In one embodiment, the oral care composition comprises:
a) an invertase selected from the group consisting of:
   a) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:3;
   b) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:12;
   c) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:15;
   d) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:18;
   e) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:21;
   f) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:24;
   g) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:27;
   h) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:30;
   i) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:33;
   j) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:36;
   k) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:39;
   l) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:42;
   m) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:45;
   n) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:48;
b) a beta-glucosidase having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:6; and
c) a glucoamylase having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:9; and, optionally,
d) an alpha-amylase having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:50.

In one embodiment, the oral care composition comprises:
a) an invertase comprising, consisting essentially of, or consisting of SEQ ID NO:3; b) a beta-glucosidase comprising, consisting essentially of, or consisting of SEQ ID NO:6;
c) a glucoamylase comprising, consisting essentially of, or consisting of SEQ ID NO:9; and, optionally
d) an alpha-amylase comprising, consisting essentially of, or consisting of SEQ ID NO:50.

The oral care ingredients may be varied according to the different types of oral care composition as well as the desired characteristics and/or activities of the oral care compositions. For the purpose of the present invention, the terms “ingredient” and “component” are used interchangeably in relation to oral care compositions.

An oral care composition of the invention may be an internal oral care composition such as toothpaste or toothpaste tablet, dental cream, mouthwash or mouthwash tablet, mouth rinse, lozenges, pastilles, chewing gum, confectionary, candy, and the like, which is designed to remove biofilm inside the oral cavity, e.g., biofilm residing on teeth, on soft tissues of the oral cavity, and on dentures residing in the oral cavity.

An oral care composition of the invention may also be an external oral care composition such as denture cleaning solution, denture cleaning tablet, denture cleaning powder, and the like, which is designed to remove biofilm from dentures that have been removed from the oral cavity for cleaning.

In a preferred embodiment, the oral care composition is an internal oral care composition, and the at least one oral care component is selected from the group consisting of abrasives, humectants, solvents, thickening agents, binding agents, buffering agents, foaming agents, foaming modulators, sweetening agents, softening agents, plasticizing agents, flavoring agents, coloring agents, therapeutic agents, anti-microbial agents, tartar-controlling agents, fluoride sources, preservatives, detergents, surfactants, coloring agents, buffering agents, softeners, plasticizers, whitening agents, bleaching agents, gum-base ingredients, and bulking agents.

Although the oral care ingredients mentioned herein are categorized by a general header according to a functionality, this is not to be construed as a limitation, as an ingredient may comprise additional functionalities as will be appreciated by the skilled person.

### Toothpaste, Dental Cream, Mouthwash, and Mouth Rinse

Internal oral care compositions of the invention in the form of toothpaste, dental cream, mouthwash, and mouth rinse may include ingredients and/or substances selected from the following categories:

| Type of ingredient | Examples | Amount (weight %) |
|---|---|---|
| Abrasives | Calcium Carbonate, Sodium bicarbonate, | 0 to 70% |
| | Diatomite, Charcoal, Dicalcium phosphate | |
| | dehydrate, Hydrated Silica, Hydrated Alumina | |
| Humectants | Glycerol, sorbitol, and propylene glycol | 0 to 80% |
| Solvents | Water, ethanol, water/ethanol mixtures | 0.001 to 95% |
| Thickening/binding | Xanthan gum, cellulose gum (sodium | 0.01 to 20% |
| agents | carboxymethyl cellulose), hydroxyethyl | |
| | cellulose, carrageenan, glycerin, | |
| | potassium thiocyanate, PVM/MA copolymer, | |
| | carbomer, arrowroot powder | |
| Buffering agents | Sodium Hydroxide, Sodium phosphate, Di- | 0.001 to 10% |
| | and trisodium phosphate, Sodium Citrate, | |
| | Calcium Glycerophosphate, | |
| Formulation and | Sodium lauryl sulfate (SLS), sodium lauryl | 0.001 to 10% |
| foaming agents | sarcosinate, sodium lauroyl glutamate, sodium | |
| (detergents, | methyl cocoyl taurate, Steareth-30, coco- | |
| surfactants) | glucoside, propylene glycol (1,2-propanediol), | |
| | Poloxamer 407, Polysorbate 20, Polysorbate 80 | |
| Foaming | PEG's (polyethylene glycols) | 0.001 to 10% |
| agents/modulators | | |
| Sweetening agents | Xylitol, Sorbitol, Erythritol, mannitol, Sodium | 0.001 to 60% |
| | Saccharin, Maltitol, Stevia, Sucralose, | |
| | Acesulfame K, aspartame, Hydrogenated | |
| | Starch Hydrolysate | |
| Softener/plasticizer | Lecithin, Hydrogenated vegetable oils, | 0.001 to 5% |
| | Glycerol ester, Lanolin, Methyl ester, | |
| | Pentaerythritol ester, Stearic Acid, sodium | |
| | and Potassium Stearates | |
| Flavoring agents | Peppermint oil, spearmint oil | 0.001 to 5% |
| Coloring agents | Titanium dioxide, beta-carotene, | 0.001 to 20% |
| | FD&C Blue 1 Aluminum Lake, FD&C Blue 2 | |
| | Lake, FD&C Yellow 5 Aluminum Lake, FD&C | |
| | Yellow 5 | |
| Preservatives | Sodium Benzoate, Methyl Paraben, Ethyl | 0.001 to 30% |
| | Paraben, Citric acid, Potassium Sorbate, | |
| | Calcium Citrate, Propylene glycol | |
| Whitening/bleaching | Hydrogen peroxide, sodium hypochlorite, | 0.01 to 10% |
| agents | sodium perborate | |
| Therapeutic agents | Calcium Fluoride, Sodium Fluoride, Triclosan, | 0.001 to 50% |
| (including anti- | Alcohol, various essential oils (Thymol, | |
| bacterial, anti-carries, | Menthol), Benzoic acid, Sodium benzoate, zinc | |
| anti-inflammatory | Gluconate, Aloe Vera, chloride, | |
| agents) | cetylpyridinuim chloride, Domiphen bromide, | |
| | Methyl salicylate, Chlorhexidine digluconate | |

### Toothpaste

Toothpastes and dental creams/gels typically include as oral care ingredients abrasives, solvents, humectants, detergents/surfactants, thickening and binding agents, buffering agents, flavoring agents, sweetening agents, fluoride sources, therapeutic agents, coloring agents, and preservatives.

In a preferred embodiment, the present invention relates to oral care compositions in the form of a toothpaste or dental cream comprising an invertase, a beta-glucosidase, a glucoamylase, and at least one oral care ingredient, wherein the at least one oral care ingredient is selected from the following ingredients:

| Type of ingredient | Examples | Amount (weight %) |
|---|---|---|
| Abrasive | Calcium carbonate, sodium | 10 to 50% |
| | bicarbonate, hydrated silica, hydrated | |
| | Alumina | |
| Solvent | Water | 5 to 40% |
| Humectant | Glycerin and sorbitol | 20 to 70% |
| Detergent/Surfactant | Sodium lauryl sulphate (SLS), sodium | 1 to 3% |
| | lauryl sarcosinate, cocamidopopybetaine | |
| | and steareth-30 | |
| Thickening and binding | Natural gums (e.g. xanthan gum, | 1 to 2% |
| agent | cellulose gum) or synthetic polymers (e.g., | |
| | PVM/MA copolymer, carbomer). hydroxyethyl | |
| | cellulose, carrageenan, glycerin. | |
| Buffering agent | Sodium citrate, calcium glycerophosphate, | 1 to 60% |
| | sodium hydroxide, di- and trisodium | |
| | pyrophosphate, disodium phosphate | |
| Flavoring agent | Peppermint oil, spearmint oil | 0 to 2% |
| Sweetening agent | Xylitol, sorbitol, erythritol, saccharin, | 0 to 2% |
| | stevia, sucralose | |
| Fluoride source | Sodium fluoride, calcium fluoride, | 0.001 to 10% |
| | stannous fluoride, sodium | |
| | monofluorophosphate | |
| Therapeutic agent | Triclosan, zinc gluconate, aloe vera | 0.001 to 50% |
| | extracts, chloride, cetylpyridinuim | |
| | chloride, domiphen bromide, methyl | |
| | salicylate, chlorhexidine, chlorhexidine | |
| | digluconate | |
| Coloring agent | Titanium dioxide | 0 to 0.5% |
| Preservative | Sodium gluconate, citric acid, sodium | 0.05 to 0.5% |
| | benzoate | |

In a preferred embodiment, the present invention relates to oral care compositions in the form of a toothpaste or dental cream comprising an invertase, a beta-glucosidase, a glucoamylase, an alpha-amylase, and at least one oral care ingredient.

An oral care composition of the invention may be a toothpaste comprising the following ingredients (in weight % of the final toothpaste composition):

Abrasive: 10 to 70%

Humectant: 0 to 80%

Thickening agent: 0.1 to 20%

Binding agent: 0.01 to 10%

Sweetening agent: 0.1 to 5%

Foaming agent: 0 to 15%

Enzymes (invertase, beta-glucosidase, glucoamylase, optionally alpha-amylase): 0.01 to 20%

### Mouthwash

Mouthwashes and mouth rinses of the invention, including plaque removing liquids, typically include as oral care ingredients a carrier liquid, detergents/surfactants, buffering agents, flavoring agents, humectants, sweetening agents, therapeutic agents, fluoride sources, coloring agents, and preservatives.

In a preferred embodiment, the present invention relates to oral care compositions in the form of a mouthwash or mouth rinse comprising an invertase, a beta-glucosidase, a glucoamylase, and at least one oral care ingredient, wherein the at least one oral care ingredient is selected from the following ingredients:

| Type of ingredient | Example |
|---|---|
| Carrier liquid, usually | Water, ethanol (18-27%), water/ethanol |
| 60-99% | mixture |
| Detergent/surfactant | Poloxamer 407, Polysorbate 20, Polysorbate |
| | 80, Steareth-30 |
| Buffering agent | Sodium Hydroxide, (Di)-Sodium phosphate, |
| | Sodium citrate, Benzoic acid, Sodium |
| | Benzoate |
| Flavoring agent | Peppermint oil, spearmint oil |
| Humectants | Glycerin, Sorbitol |
| Sweetening agents | Saccharin, Xylitol |
| Therapeutic agent | Triclosan, zinc gluconate, aloe vera extracts, |
| | chloride, cetylpyridinuim chloride, domiphen |
| | bromide, methyl salicylate, chlorhexidine, |
| | chlorhexidine digluconate |
| Fluoride source | Sodium fluoride, calcium fluoride, stannous |
| | fluoride, or sodium monofluorophosphate |
| Coloring agent | Titanium oxide |
| Preservative | Sodium Benzoate, Benzoic acid, Citric acid, |
| | Propylene glycol |

In a preferred embodiment, the present invention relates to oral care compositions in the form of a mouthwash or mouth rinse comprising an invertase, a beta-glucosidase, a glucoamylase, an alpha-amylase, and at least one oral care ingredient.

An oral care composition of the invention may be a mouthwash comprising the following ingredients (in weight % of the final mouthwash composition):

Water: 0 to 70%

Ethanol: 0 to 20%

Humectant: 0 to 20%

Surfactant: 0 to 2%

Enzymes (invertase, beta-glucosidase, glucoamylase, optionally alpha-amylase): 0.01 to 20%

Other ingredients: 0 to 2% (e.g., flavors, sweeteners, fluoride sources).

The mouthwash composition may be buffered with an appropriate buffer, e.g., sodium citrate or phosphate in the pH range 6-7.5.

Relevant oral care components suitable for toothpastes, dental creams, mouthwashes, and mouth rinses is further detailed below. The skilled person is capable of varying the oral care components according to the type of oral care composition as well as the desired characteristics and/or activities of the specific oral care composition. An oral care composition may not necessarily comprise all the mentioned ingredients.

### Abrasives

Abrasive polishing material might be incorporated into the oral care composition of the invention. According to the invention said abrasive polishing material includes alumina and hydrates thereof, such as alpha alumina trihydrate, magnesium trisilicate, magnesium carbonate, kaolin, aluminosilicates, such as calcined aluminum silicate and aluminum silicate, calcium carbonate, zirconium silicate, bentonite, silicium dioxide, sodium bicarbonate, and also powdered plastics, such as polyvinyl chloride, polyamides, polymethyl methacrylate, polystyrene, phenol-formaldehyde resins, melamine-formaldehyde resins, urea-formaldehyde resins, epoxy resins, powdered polyethylene, silica xerogels, hydrogels and aerogels, and the like.

Also suitable as abrasive agents are calcium pyrophosphate, water-insoluble alkali metaphosphates, poly-metaphosphates, dicalcium phosphate and/or its dihydrate, dicalcium orthophosphate, tricalcium phosphate, particulate hydroxyapatite, and the like. It is also possible to employ mixtures of these substances.

Silica dental abrasives of various types are preferred because of their unique benefits of exceptional dental cleaning and polishing performance without unduly abrading tooth enamel or dentine, and which have a good compatibility with other possible ingredients, like metal ions and fluoride.

Dependent on the oral care composition, the abrasive product may be present in from 0 to 70% by weight, preferably from 1% to 70%.

For toothpastes the abrasive material content typically lies in the range of from 10% to 70% by weight of the final tooth-paste product.

### Humectants

Humectants are employed to prevent loss of water from, e.g., toothpastes and to avoid hardening of toothpastes upon exposure to air. Some humectants also give a desirable sweetness of flavor to toothpaste and mouthwash compositions. Suitable humectants for use in oral care compositions according to the invention include the following compounds and mixtures thereof: glycerol, polyol, sorbitol, xylitol, maltitol, lactitol, polyoxyethylene, polyethylene glycols (PEG), polypropylene glycols, propylene glycol, 1,3-propanediol, 1,4-butanediol, hydrogenated partially hydrolyzed polysaccharides and the like, coconut fatty acid, amide of N-methyl-taurine, and Pluronic®.

Humectants are in generally present in from 0% to 80%, preferably 5 to 70% by weight.

### Thickening/Binding Agents

Suitable thickening and/or binding agents include silica, starch, tragacanth gum, xanthan gum, karaya gum, carrageenans (extracts of Irish moss), gum arabic, alginates, pectin, cellulose derivatives, such as hydroxyethyl cellulose, sodium carboxymethyl cellulose, hydroxypropyl cellulose and hydroxyethyl propyl cellulose, polyacrylic acid and its salts, polyvinylpyrrolidone and carboxyvinyl polymers, as well as inorganic thickeners such as amorphous silica compounds. These agents stabilize the oral care compositions of the invention.

Thickeners may be present in toothpaste, dental creams and gels as well as in mouthwashes in an amount of from 0.1 to 20% by weight, and binders to the extent of from 0.01 to 10% by weight of the final product.

### Foaming Agents and Foaming Modulators

As foaming agent soap, anionic, cationic, non-ionic, amphoteric and/or zwitterionic surfactants can be used, either alone or in combinations. These may be present at levels of from 0% to 15%, preferably from 0.1% to 13%, more preferably from 0.25% to 10% by weight of the final product. Surfactants are only suitable to the extent that they do not exert an inactivation effect on the enzymes and other components included in the oral care composition. Useful surface-active agents include anionic, nonionic, and ampholytic compounds, with anionic compounds being preferred.

Examples of suitable surfactants include salts of the higher alkyl sulfates, such as sodium lauryl sulfate or other suitable alkyl sulfates having 8 to 18 carbon atoms in the alkyl group; sodium lauryl sulfoacetate, salts of sulfonated monoglycerides of higher fatty acids, such as sodium coconut monoglyceride sulfonate or other suitable sulfonated monoglycerides of fatty acids of 10 to 18 carbon atoms; salts of amides of higher fatty acid, e.g., 12 to 16 carbon atom acids, with lower aliphatic amino acids, such as sodium-N-methyl-N-palmitoyl tauride, sodium N-lauroyl-, N-myristoyl- and N-palmitoyl sarcosinates; salts of the esters of such fatty acids with isotopic acid or with glycerol monosulfate; such as the sodium salt of monosulfated monoglyceride of hydrogenated coconut oil fatty acids; salts of olefin sulfonates, e.g., alkene sulfonates or alkene sulfonates or mixtures thereof having 12 to 16 carbon atoms in the carbon chain of the molecule; and soaps of higher fatty acids, such as those of 12 to 18 carbon atoms, e.g., coconut fatty acids.

The cation of the salt may be sodium, potassium or mono-, di or triethanol amine. The nonionic surfactants include sucrose/fatty acid esters, maltose/fatty acid esters, maltitol/fatty acid esters, maltotriitol/fatty acid esters, maltotetraitol/fatty acid esters, maltopentaitol/fatty acid esters, maltohexaitol/fatty acid esters, mahoheptaitol/fatty acid esters, sorbitan/fatty acid esters, lactose/fatty acid esters, lactinose/fatty acid esters, polyoxyethylene/polyoxypropylene copolymers, polyoxyethylene alkyl ethers, polyoxyethylene/fatty acid esters, fatty acid alkanolamides, polyoxyethylene sorbitan/fatty acid esters, polyoxyethylene/hydrogenated castor oil, and polyglycerin/fatty acid esters.

Most preferred are sodium lauryl sulphate, sodium dodecylbenzene sulphonate and sodium lauryl sarcosinate.

Preferred foaming modulators include polyethylene glycols.

Foaming agents and foaming modulators may be present from in an amount of from 0% to 15% by weight, preferably from 0.01% to 10% by weight.

### Sweetening Agents

Suitable sweeteners include, but are not limited to, saccharin and water-soluble salts thereof, dextrose, sucrose, lactose, maltose, levulose, aspartame, cyclamate salts, D-tryptophan, dihydrochalchones, acesulphame, stevioside, levaudioside, glycyrrhizins, pellartine, thaumatin, p-methoxycinnamic aldehyde, hydrogenated starch hydrolysates, xylitol, sorbitol, erythritol, mannitol, and mixtures thereof.

Sweeteners may be present from in an amount of from 0.001% to 60% by weight, preferably from 0.01% to 50% by weight.

### Flavoring Agents

Flavoring agents are usually present in low amounts, such as from 0.01% to about 5% by weight, especially from 0.1% to 5%. The flavors that may be used in the invention include, but are not limited to, wintergreen oil, peppermint oil, spearmint oil, clove bud oil, menthol, anethole, methyl salicylate, eucalyptol, cassia, 1-inenthvl acetate, sage, eugenol, parsley oil, oxanone, alpha-irisone, marjoram, lemon, orange, cranberry, propenyl guaethol, cinnamon, vanillin, ethyl vanillin, heliotropine, 4-cis-heptenal, diacetyl, methylpara-tert-butyl phenyl acetate, carvone, cineole, menthone, cinnamic aldehyde, limonene, ocimene, n-decyl alcohol, citronellol, alpha-terpineol, methyl acetate, citronellyl acetate, methyl eugenol, linalool, thymol, rosemary oil, pimento oil, diatomaceous oil, eucalyptus oil, and mixtures thereof.

Coolants may also be part of the flavor system or added separately to the composition. Preferred coolants in the present compositions are the paramenthan carboxyamide agents such as N-ethyl-p-menthan-3-carboxamide (known commercially as ‘WS-3”), menthol, 3-1-menthoxypropanc-1,2-diol (“TK-10”), menthone glycerol acetal (“MGA”), menthyl lactate and mixtures thereof.

### Whitening/Bleaching Agents

Whitening/bleaching agents include H₂O₂ and may be added in amounts less than 5%, preferably from 0.05 to 4%, calculated on the basis of the weight of the final composition.

Other bleaching components which might be comprised by the present invention include, peroxydiphosphate, urea, peroxide, metal peroxides such as calcium peroxide, sodium peroxide, stronthium peroxide, magnesium peroxide, hypochlorite salts such as sodium hypochlorite, and the salts of perborate, persilicate, perphosphate and percarbonate such as sodium perborate, potassium persilicate and sodium percarbonate. The peroxide compounds can be stabilized by addition of a triphenylmethane dye, a chelating agent or antioxidants such as butylated hydroxy anisole (BHA) or butylated hydroxy toluene (BHT).

### Solvent

A solvent is usually added to compositions of the invention in an amount sufficient for giving the compositions a flowable form in case the compositions is; e.g., a toothpaste, dental cream, or gel, or to dissolve the other components of a compositions, in case of, e.g., a mouthwash or mouth rinse.

Suitable solvents include water, ethanol, and water/ethanol mixtures, which may be present in an amount of from 0.1% to 70%.

### Anti-Microbial Agents

The present invention also includes water-soluble anti-microbial agents, such as chlorhexidine, triclosan, digluconate, hexetidine, alexidine, quaternary ammonium antibacterial compounds, and water-soluble sources of certain metal ions such as zinc, copper, silver and stannous (e.g., zinc, copper and stannous chloride, and silver nitrate) may also be included.

Sparingly soluble zinc salts such as zinc citrate, zinc C14-alkyl maleate, zinc benzoate, zinc caproate, zinc carbonate might also be included used in the compositions of the present invention to prolong the anti-microbial effectiveness of zinc ions due to the slow dissolution of these zinc salts in saliva.

Anti-microbial agents may be present in an amount of from 0% to 50% by weight, preferably from 0.01% to 40% by weight, most preferably from 0.1% to 30% by weight.

### Tartar-Controlling Agent

Compositions of the invention may comprise a tartar-controlling agent such as inorganic phosphorous tartar-controlling agents including any of the pyrophosphates such as disodium pyrophosphate, dipotassium pyrophosphate, tetrapotassium pyrophosphate, tetrasodium pyrophosphate, and mixtures thereof.

Organic phosphorous compounds that may serve as tartar-controlling agents include polyphosphonates such as disodium ethane-1-hydroxy-1, 1-diphosphonate (EHDP), methanediphosphonic acid, and 2-phosphonobutane-1 2,4-tricarboxylic acid.

Tartar-controlling agents may be present in an amount of from 0% to 10% by weight, preferably from 0.1% to 5% by weight.

### Preservatives

Suitable preservatives include sodium benzoate, potassium sorbate, p-hydroxybenzoate esters, methyl paraben, ethyl paraben, propyl paraben, citric acid, calcium citrate, and mixtures thereof.

Preservatives may be present in an amount of from 0% to 40% by weight, preferably from 0.01% to 30% by weight.

### Fluoride Sources

Compositions of the invention may also comprise ingredients that can be used as fluoride source. Preferred soluble fluoride sources include sodium fluoride, potassium fluoride, stannous fluoride, indium fluoride, sodium monofluorophosphate, sodium hexafluorosilicate, zinc fluoride, lithium fluoride, aluminum fluoride, acidulated phosphate fluoride, ammonium bifluoride, titanium tetrafluoride, and amine fluoride.

Especially preferred are sodium fluoride and sodium monofluorophosphate.

Fluoride sources may be present in an amount of from 0% to 20% by weight, preferably from 0.01% to 15% by weight, most preferably from 0.1% to 10% by weight.

In a preferred embodiment, the at least one oral care ingredient is a fluoride source; preferably the fluoride source is selected from the group consisting of sodium fluoride, calcium fluoride, stannous fluoride, or sodium monofluorophosphate

### Coloring Agents

Coloring agents or pigments suitable for oral care compositions of the invention include nontoxic, water-insoluble inorganic pigments such as titanium dioxide and chromium oxide greens, ultramarine blues and pinks and ferric oxides as well as water insoluble dye lakes prepared by extending calcium or aluminum salts of FD&C dyes on alumina such as FD&C Green No.1 lake, FD&C Blue No.2 lake, FD&C Red No. 30 lake, FD&C Yellow No. 16 lake, and FD&C Yellow No. 10.

A preferred opacifier is titanium dioxide.

Coloring agents may be present in an amount of from 0% to 20% by weight, preferably from 0.01% to 15% by weight, most preferably from 0.1% to 10% by weight.

### Buffering Agents

The oral care compositions of present invention may also include buffering agents, i.e., pH-adjusting agents, such as alkali metal hydroxides, carbonates, sesquicarbonates, borates, silicates, phosphates, imidazole, and mixtures thereof.

Specific buffering agents include monosodium phosphate, trisodium phosphate, sodium hydroxide, potassium hydroxide, alkali metal carbonate salts, sodium carbonate, imidazole, pyrophosphate salts, sodium citrate, hydrochloric acid, sodium hydroxide, triethanolamine, triethylamine, lactic acid, malic acid, fumaric acid, tartaric acid, phosphoric acid, and mixtures of these.

Buffering agents may be present in an amount of from 0% to 10% by weight, preferably from 0.01% to 5% by weight.

### Chewing Gum

When the oral composition according to the invention is a chewing gum, it can be any known type of chewing gum, such as chewing gum pieces optionally coated, as well as sticks or chewing gum provided with an arbitrary desired shape in response to the intended use. The chewing gum preparation can be of any quality including the bubble gum quality.

In a preferred embodiment, the present invention relates to oral care compositions in the form of a chewing gum comprising an invertase, a beta-glucosidase, a glucoamylase, and at least one oral care ingredient, wherein the at least one oral care ingredient is selected from elastomer, softening agent, plasticizing agent, emulsifier, wax, coloring agent, sweetening agent, flavoring agent, bulking agent, and thickening agent. In a preferred embodiment, the oral care composition further comprises an alpha-amylase.

### Gum Base Ingredients

Chewing gum is traditionally considered as being comprised of a water-insoluble or base portion and a water-soluble portion that contains flavoring agents, sweetening agents, and coloring agents. The gum base part of the gum is a masticatory substance which imparts the chew characteristics to the final product. It defines the release profile of flavors and the sweeteners and plays a significant role in the gum product. The flavors, sweeteners and colors can be thought of as providing the sensory appeal aspects of the chewing gum. No limitations as to the chewing gum bases used in a chewing gum preparation according to the invention exist. Conventional chewing gum bases available for instance from Dansk Tyggegummi Fabrik A/S, L.A. Dreyfus or Cafasa Gum SIA, are usually suitable, but specially made formulations can also be used. The formulation depends on the desired type of chewing gum or the desired type of structure. Suitable raw materials for gum bases include the substances according to the U.S. Chewing Gum Base Regulations—Code of Federal Regulations, Title 21, Section 172,615 and in accordance with other national and international lists (or positive lists) and include elastomers, resins, waxes, polyvinyl acetates, oils, fats, emulsifiers, fillers, and antioxidants.

The gum base usually comprises from 15 to 90% by weight, preferably from 30 to 40% by weight, more preferably from 5 to 25% of the final product.

Elastomers provide the chew, springiness or bounce to the base and control bubble and flavor release in the final chewing gum. They may be any water-insoluble polymer known in the art. They include styrene butadiene copolymers (SBR) and non-SBR types, both natural and synthetic. Examples of natural elastomers include, without limitation, rubbers such as rubber latex (natural rubber) and guayule, and gums such as chicle, jelutong, balata, guttapercha, lechi capsi, sorva, crown gum, nispero, rosidinha, perillo, niger gutta, tunu, gutta kay, pendare, leche de vaca, chiquibul, crown gum, and the like, and mixtures thereof. Examples of synthetic elastomers include, without limitation, polyisobutylene, isobutylene-isoprene copolymers (butylrubber), polyethylene, polybutadiene, styrenebutadiene copolymers, polyisoprene, and the like, and mixtures thereof.

The amounts of elastomer (rubbers) employed in the gum base composition will vary greatly depending upon various factors such as the type of gum base used (adhesive, or conventional, bubble or standard) the consistency of the gum base composition desired, and the other components used in the composition to make the final chewing gum product. In general, the elastomer is present in the gum base composition in an amount of from about 15% to about 60%, preferably from about 25% to about 30%, by weight based on the total weight of the gum base composition.

Elastomer solvents aid in softening or plasticizing the elastomer component. In doing so they provide a bulkiness to the chew.

Elastomer solvents include, but are not limited to, natural rosin esters and synthetic derivatives of, e.g., terpenes. Examples of elastomer solvents suitable for use herein include tall oil rosin ester; partially hydrogenated wood and gum rosin; the glycerol esters of wood and gum rosin, partially hydrogenated wood/gum rosin, partially dimerized wood and gum rosin, polymerized wood and gum rosin, and tall oil rosin; the deodorized glycerol ester of wood rosin; the pentaerythritol esters of wood and gum rosin; partially hydrogenated wood and gum rosin; the methyl ester of partially hydrogenated wood rosin; methyl, glycerol and pentaerythritol esters of rosins and modified rosins such as hydrogenated, dimerized and polymerized rosins; terpene resins such as polymers of alpha-pinene or beta-pinene, terpene hydrocarbon resins; polyterpene; and the like, and mixtures thereof. The elastomer solvent may be employed in the gum base composition in an amount of from about 2% to about 40%, and preferably from about 7% to about 15% by weight of the gum base composition.

Polyvinyl acetates provide stretch or elasticity to the gum base. They also affect chew bulkiness, softness and bubble, hydrophilic character, and flavor release.

The amounts of the different molecular weight polyvinyl acetates present in the gum base composition should be effective to provide the finished chewing gum with the desired chew properties, such as integrity, softness, chew bulkiness, film-forming characteristic, hydrophilic character, and flavor release. The total amount of polyvinyl acetate used in the gum base composition is usually from about 45% to about 92% by weight based on the total gum base composition. The vinyl polymers may possess a molecular weight ranging from about 2000 Da up to about 95,000 Da.

Typically, the low molecular weight polyvinyl acetate has a weight average molecular weight of from about 2,000 Da to about 14,000 Da. The medium molecular weight polyvinyl acetate typically has a weight average molecular weight of from about 15,000 Da to 55,000 Da. The high molecular weight polyvinyl acetate typically has a weight average molecular weight of from 55,000 Da to about 95,000 Da but may range as high as 500,000 Da.

Waxes, fats, and oils plasticize the elastomer mixture and improve the elasticity of the gum base. Waxes can provide a soft or firm chew, affect the flavor release, and provide bulkiness and smoothness to the gum base. Fats and oils provide a soft chew. The fats, oils and waxes may be use individually or in combination or the gum base may be a wax free gum base.

Waxes when used, may be of mineral, animal vegetable or synthetic origin. Non-limiting examples of mineral waxes include petroleum waxes such as paraffin and microcrystalline waxes, animal waxes include beeswax, vegetable waxes include carnauba, candellila, rice bran, esparto, flax and sugarcane, and synthetic waxes include those produced by the Fischer-Tropsch synthesis, and mixtures thereof.

Suitable oils and fats usable in gum compositions include hydrogenated or partially hydrogenated vegetable or animal fats, such as cottonseed oil, soybean oil, coconut oil, palm kernel oil, beef tallow, hydrogenated tallow, lard, cocoa butter, lanolin, and the like; fatty acids such as palmitic, oleic, stearic, linoleic, lauric, myristic, caproic, caprylic, decanoic or esters and salts as sodium stearate and potassium stearate. These ingredients when used are generally present in amounts up to about 7% by weight of the gum composition, and preferably up to about 3.5% by weight of the gum composition.

Preferred as softeners are the hydrogenated vegetable oils and include soybean oil and cottonseed oil which may be employed alone or in combination. These softeners provide the gum base composition with good texture and soft chew characteristics. These softeners are generally employed in an amount from about 5% to about 14% by weight of the gum base composition.

Emulsifiers aid in dispersing the immiscible components of the gum base composition into a single stable system. They provide hydrophilic character to a gum base and aid in plasticizing the resins and polyvinyl acetates. They also affect the softness of the base and the bubble character of the base. Typical emulsifiers include acetylated monoglyceride, glyceryl monostearate, lecithin, fatty acid monoglycerides, diglycerides, propylene glycol monostearate, lecithin, triacetin, glyceryl triacetate and the like, and mixtures thereof.

Preferred emulsifiers are glyceryl monostearate and acetylated monogylcerides. These serve as plasticizing agents. The emulsifiers may be employed in an amount of from about 2% to about 15% by weight of the gum base composition, and preferably from about 7% to about 11% by weight of the gum base composition.

The fats, oils, waxes, emulsifiers, and certain sugar bulking agents are often grouped together and referred to as softening agents. Because of the low molecular weight of these ingredients, the softeners can penetrate the fundamental structure of the gum base making it plastic and less viscous. Useful plasticizers and softeners of the above include lanolin, palmitic acid, oleic acid, stearic acid, sodium stearate, potassium stearate, glyceryl triacetate, glyceryl lecithin, glyceryl monostearate, propylene glycol nonastearate, acetylated monoglyceride, glycerin, fully unsaturated vegetable oils such as nonhydrogenated cottonseed oil, hydrogenated vegetable oils, petroleum waxes, sorbitan monostearate, tallow, and the like, and mixtures thereof and also include high fructose corn syrup, corn syrup, sorbitol solution, hydrogenated starch hydrolysate, and the like, and mixtures thereof.

The amount of softener present should he an effective amount to provide a finished chewing gum with the desired chew bulkiness and softness. When used as softeners these materials are generally employed in the gum base composition in an amount of up to about 25%, and preferably in an amount of from about 1% to about 17%, by weight of the gum base composition.

The gum base may further contain a surfactant. Examples of suitable surfactants include polyoxyethylene (20) sorbitan monoleate, polyoxyethylene (20) sorbitan monolaurate, polyethylene (4) sorbitan monolaurate, polyoxyethylene (20) sorbitan monopalmitate, polyoxyethylene (20) sorbitan monostearate, polyoxyethylene, (4) sorbitan monostearate, polyoxyethylene (20) sorbitan tristearate, polyoxyethylene (5) sorbitan monooleate, polyoxyethylene (20) sorbitan trioleate, sorbitan monolaurate, and the like. The amount of surfactant present should be effective to provide the finished chewing gum with the desired softness. Typically, the surfactant is employed in the base in an amount of from about 0.5% to about 3.0% by weight based on the total weight of the gum base.

The gum base composition of this invention may also include effective amounts of fillers sometimes referred to as bulking agents. These materials add firmness and bulk and affect the texture and the flavor release of the chewing gum. Useful fillers include organic and inorganic compounds (mineral adjuvants) such as calcium carbonate, magnesium carbonate, ground limestone, magnesium silicate, calcium phosphate, cellulose polymers, clay, alumina, aluminum hydroxide, aluminum silicate, tale, tricalcium phosphate, dicalcium phosphate, and the like, and mixtures thereof. These fillers or adjuvants may be used in the gum base compositions in various amounts. The amount of the filler present should be effective to provide a finished chewing gum with the desired flavor release and integrity. Typically, the filler is employed in the gum base composition in an amount from about 1% to about 40%, and preferably from about 5% to about 20%, by weight of the gum base composition.

The gum base may also comprise an antioxidant to provide improved stability, lessen any oil-taste and provide longer shelf life. Typical non-limiting examples of antioxidants are butylated hydroxytoluene (BHT), butylated hydroxy anisole (BHA), propyl gallate. Mixtures thereof may also be used.

### Other Gum Ingredients

The remaining ingredients in chewing gum compositions are conventional and usually comprise from 10 to 85% by weight of the final product.

Examples thereof are sweetening agents, softeners, coloring agents, bulking agents, thickening agents, and flavoring agents of the type and in the amounts conventionally used for chewing gum.

Suitable flavoring agents those flavors known to the skilled artisan such as natural and artificial flavors. These flavorings may be chosen from synthetic flavor oils and flavoring aromatics and/or oils, oleoresins and extracts derived from plants, leaves, flowers, fruits, and so forth, and combinations thereof. Non-limiting representative flavor oils include spearmint oil, cinnamon oil, wintergreen oil (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Other useful flavorings are artificial, natural, and synthetic fruit flavors such as vanilla, and citrus oils including lemon, orange, lime, grapefruit, and fruit essences including apple, pear, peach, grape, strawberry, raspberry, cherry, plum, pineapple, apricot and so forth. These flavoring agents may be used in liquid or solid form and may be used individually or in admixtures. Commonly used flavors include mints such as peppermint, menthol, artificial vanilla, cinnamon derivatives, and various fruit flavors, whether employed individually or in admixture.

Other useful flavoring agents include aldehydes and esters such as cinnamyl acetate, cinnamaldehyde, citrate diethylacetal, dihydrocarvyl acetate, eugenyl formate, p-methyl anisole, and so forth may be used. Generally, any flavoring or food additive may be used.

Further examples of aldehyde flavorings include, but are not limited to, acetaldehyde (apple), benzaldehyde (cherry, almond), anisic aldehyde (licorice, anise), cinnamic aldehyde (cinnamon), citral, i.e., alpha-citral (lemon, lime), neral, i.e., beta-citral (lemon, lime), decanal (orange, lemon), ethyl vanillin (vanilla, cream), heliotrope, i.e., piperonal (vanilla, cream), vanillin (vanilla, cream), alpha-amyl cinnamaldehyde (spicy fruity flavors), butyraldehyde (butter, cheese), valeraldehyde (butter, cheese), citronellal (many types), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e., trans-2-hexenal (berry fruits), tolyl aldehyde (cherry, almond), veratraldehyde (vanilla), 2,6-dimethyl-5-heptenal, i.e., melonal (melon), 2,6-dimethyloctanal (green fruit), and 2-dodecenal (citrus, mandarin), cherry, grape, strawberry shortcake, mixtures thereof and the like.

The amount of flavoring agent employed herein is normally a matter of preference subject to such factors as the type of final chewing gum composition, the individual flavor, the gum employed, and the strength of flavor desired. Thus, the amount of flavoring may be varied to obtain the result desired in the final product and such variations are within the capabilities of those skilled in the art without the need for undue experimentation. In gum compositions, the flavoring agent is generally present in amounts from about 0.02% to about 5% by weight of the chewing gum composition.

The chewing gum compositions generally include bulking agents. These bulking agents (carders, extenders) may be water-soluble and include bulking agents selected from the group consisting of, but not limited to, monosaccharides, disaccharides, polysaccharides, sugar alcohols, and mixtures thereof; sorbitol, xylitol, maltitol, mannitol, isomalt (a racemic mixture of alphaD-glucopyranosyl-1,6-mannitol and alpha-D-glucopyranosyl-1,6-sorbitol manufactured under the tradename Palatinit™ by Suddeutsche Zucker), glycerol, aspartame, Lycasin® glycerol, galactitol acesulphame K, saccharine and salts thereof, cyclamate and salts thereof, neohesperidine dihydrochalcone, glycyrrhizinic acid and salts thereof, thaumantine and sucralose as well as mixtures thereof or mixtures thereof with other suitable sweeteners, maltodextrins; hydrogenated starch hydrolysates; hydrogenated hexoses; hydrogenated disaccharides; minerals, such as calcium carbonate, talc, titanium dioxide, dicalcium phosphate, celluloses and the and the like, and mixtures thereof. Bulking agents may be used in amounts up to about 60%, and preferably in amounts from about 25% to about 60%, by weight of the chewing gum composition.

The chewing gum compositions may also include a high intensity sweetening agent (sweeteners). High intensity sweetening agents have a sweetness intensity substantially greater than that of sucrose. Examples of suitable intense sweeteners include:
a) water-soluble naturally occurring intense sweeteners such as dihydrochalcones, monellin, steviosides, glycyrrhizin, dihydroflavenol, and L-aminodicarboxylic acid aminoalkonoic acid ester amides, such as those disclosed in in U.S. Pat. No. 4,619,834, and mixtures thereof;
b) water-soluble artificial sweeteners including the soluble saccharin salts such as sodium or calcium saccharin salts, cyclamate salts, the sodium, ammonium or calcium salts of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (Acesulfam-K), the free acid form of saccharin, and the like, and mixtures thereof;
c) dipeptide based sweeteners including L-aspartic acid derived sweeteners such as 1-aspartyl-L-phenylalanine methyl ester (Aspartame) and materials described in U.S. Pat. No. 3,492,131, L-alpha-aspartyl-N-(2,2,4,4-tetramethyl-3-thietanyl)-D-alaninamide hydrate (Alitame), methyl esters of L-aspartyl-L-phenylglycerine and L-aspartyl-L-2,5-dihydrophenyl-glycine, L-aspartyl-2.5-dihydro-L-phenylalanine, L-aspartyl-L-(1-cyclohexen)-alanine, and the like, and mixtures thereof;
d) water-soluble intense, sweeteners derived from naturally-occurring water-soluble sweeteners, such as chlorinated derivatives of ordinary sugar (sucrose), e.g., chlorodeoxysugar derivatives such as derivatives of chlorodeoxysucrose or chlorodeoxygalactosucrose, known, for example, under the product designation of Sucralose®; examples of chlorodeoxysucrose and chlorodeoxygalactosucrose derivatives include but are not limited to: to 1-chloro-1′-deoxysucrose; 4-chloro-4-deoxy-alpha-D-galactopyranosyl-alpha-D-fructofuranoside, or 4-chloro-4-deoxygalactosucrose; 4-chloro-4-deoxy-alpha-D-galactopyranosyl-1-chloro-Ideoxy-beta-D-fructofuranoside, or 4,1′-dichloro-4,1′-dideoxygalactosucrose; 1′,6′-dichloro-1′,6′-dideoxysucrose; 4-chloro-4-deoxy-alpha-D-galactopyranosyl-1,6-dichloro-1,6-dideoxy-beta-D-fructofuranoside, or 4,1′,6′-trichloro-4,1′,6′-trideoxygalactosucrose; 4,6-dichloro-4,6-dideoxy-alpha-D-galactopyranosyl-6-chloro-6-deoxy-beta-D-fructofuranoside, or 4,6,6′-trichloro-4,6,6′-trideoxygalactosucrose; 6,1′,6′-trichloro-6,1′,6′-trideoxysucrose; 4,6-dichloro-4,6-dideoxy-alpha-D-galactopyranosyl-1,6-dichloro-1,6-dideoxy-beta-D-fluctofuranoside, or 4,6,1′,6′-tetrachloro-4,6,1′,6′-tetradeoxygalactosucrose; and 4,6,1′,6′-tetradeoxy-sucrose, and mixtures thereof; and
e) protein based intense sweeteners such as Thaumaoccous daniclii (Thaumatin I and II). The amount of sweetener employed in the chewing gum composition will vary with the sweetener selected for a particular chewing gum. Thus, for any given sweetener, a sufficient amount of sweetener is used to provide the level of sweetness desired. The saccharide sweeteners and sugar alcohols described above are usually used in an amount of from about 1% to about 70% and preferably in an amount of from about 40% to about 50%, by weight based on the total weight of the chewing gum composition. The intense sweeteners described above are usually used in an amount of up to about 1%, preferably from about 0.05% to about 0.4%, by weight based on the total weight of the chewing gum composition.

The coloring agents useful in the present invention are used in amounts effective to produce the desired color. These coloring agents include pigments, which may be incorporated in amounts up to about 6%, by weight of the gum composition. A preferred pigment, titanium dioxide, may be incorporated in amounts up to about 2%, and preferably less than about 1%, by weight of the gum composition. The colorants may also include natural food colors and dyes suitable for food, drug, and cosmetic applications. These colorants are known as F.D.& C. dyes and lakes. The materials acceptable for the foregoing uses are preferably water-soluble. Illustrative non-limiting examples include the indigoid dye known as F.D.& C. Blue No.2, which is the disodium salt of 5,5-indigotindisulfonic acid. Similarly, the dye known as F.D.& C. Green No.1 comprises a triphenylmethane dye and is the monosodium salt of 4-[4-(N-ethyl-N-p-sulfoniumbenzylamino)diphenylmethylene]-[1-(N-ethyl-N-p-sulfoniumbenzyl)-delta-2,5-cyclo-hexadieneimine].

Examples of thickening agents include methyl cellulose, alginates, carrageenan, xanthan gum, gelatin, carob, tragacanth, and locust bean, emulsifiers, such as lecithin and glyceryl monostearate, acidulants such as malic acid, adipic acid, citric acid, tartaric acid, fumaric acid, and mixtures thereof.

The plasticizers, softening agents, emulsifiers, waxes, and antioxidants discussed above as being suitable for use in the gum base may also be used in the chewing gum composition.

### Active Gum Ingredients

Oral care compositions of the invention in the form of a chewing gum may also contain various active ingredients such as antimicrobial agents, Zn salts, fluorides, and urea.

Moreover, the oral composition according to the invention may, if desired, include any other active ingredients, such as anti-caries agents, anti-calculus agents, anti-plaque agents, anti-periodontal agents, anti-fungal agents, anti-smoking agents, anti-cold agents, agents against gingivitis, etc.

The antimicrobials used in the compositions can be any of a wide of cationic antimicrobial agents such as quaternary ammonium compounds (e.g., cetyl pyridinium chloride) and substituted guanidines such as chlorhexidine and the corresponding compound alexidine. Mixtures of cationic anti-microbials may also be used in the present invention.

Antimicrobial quaternary ammonium compounds include those in which one or two of the substituents on the quaternary nitrogen has a carbon chain length (typically alkyl group) of some 8 to 20, typically 10 to 18 carbon atoms while the remaining substituents (typically alkyl or benzyl group) have a lower number of carbon atoms, such as 1 to 7 carbon atoms, typically methyl or ethyl groups. Dodecyl trimethyl ammonium bromide, tetradecyl pyridinium chloride, tetradecyl ethyl pyridinium chloride, dodecyl dimethyl (2-phenoxyethyl) ammonium bromide, benzyl dimethylstearyl ammonium chloride, cetyl pyridinium chloride, quaternized 5-amino-1,3-bis 2-ethylhexyl)-5-methyl hexa hydropyrimidine and benzethonium chloride are exemplary of typical quaternary ammonium antibacterial agents. Other compounds are the bis[4-(R-amino)-1-pyridinium]alkanes as disclosed in U.S. Pat. No. 4,206,215, Jun. 3, 1980, to Bailey incorporated herein by reference. The pyridinium compounds are the preferred quaternary ammonium compounds.

The cationic antimicrobial is generally used in the present compositions at a level of from about 0.02% to about 1%, preferably from about 0.3% to about 0.7% most preferably from about 0.3% to about 0.5%.

As easily soluble zinc salt it is in principle possible to use any physiologically acceptable, easily soluble zinc salt of an inorganic or organic acid, said salt being able to release zinc ions and being approved for the intended use, such as in foodstuffs, cosmetics, or pharmaceutical products. Non-limiting examples are for instance zinc citrate, zinc sulphate, zinc lactate, zinc chloride, zinc acetate as well as mixtures thereof. Among these salts zinc acetate is preferred.

The zinc salt used must be easily soluble such that a release is ensured in the oral cavity of an amount of zinc ions efficient for the purpose aimed at within a suitable period of time.

Advantageously, the zinc salt is present in the oral composition in an amount of from 0.001 to 1.25% by weight. The amount used depends on the administration form and the intended use and is adapted such that an amount of zinc ions efficient for the intended use is released.

As taste-masking salt is used at least one salt selected among sodium chloride, ammonium chloride and physiologically acceptable alkali metal, alkaline earth metal and/or ammonium carbonates.

The alkali metal is in particular sodium or potassium, whereas the alkaline earth metal advantageously is calcium or magnesium. Particularly preferred taste-masking salts are sodium, potassium and magnesium carbonates, sodium chloride, ammonium chloride as well as mixtures thereof.

The taste-masking salt is advantageously used in the oral composition in an amount of from 0.05 to 6.25% by weight, more preferred from 0.25 to 3.50% by weight, such as from 0.50 to 2.50% by weight.

The amount used of taste-masking salt for masking the taste of zinc can in each case be determined by a person skilled in the art and depends on the particular zinc salt in question and the selected administration form.

Urea is used as an anticariogenic product for neutralizing the acid produced in dental plaque after eating or drinking. Beyond urea the composition also can contain pharmacologically acceptable substances capable of releasing urea under the conditions prevailing in the mouth. Examples thereof are salts and addition compounds between urea and inorganic compounds such as magnesium sulphate, calcium phosphate, sodium chloride, etc.

The urea content of the composition according to the invention varies between 0.05% by weight and 80% by weight, preferably between 0.2% by weight and 25% by weight.

The chewing gum compositions may be prepared using standard techniques and equipment known to those skilled in the art. The apparatus useful in accordance with the present invention comprises mixing and beating apparatus as well.

### Lozenges and Pastilles

Lozenges are flavored medicated dosage forms intended to be sucked and held in the mouth or pharynx. They may contain vitamins, antibiotics, antiseptics, local anesthetics, antihistamines, decongestants, corticosteroids, astringents, analgesics, aromatics, demulcents, or combinations of these ingredients. Lozenges may take various shapes, the most common being the flat, circular, octagonal, and biconvex forms. Another type, called bacilli, are in the form of short rods or cylinders. A soft variety of lozenge, called a pastille, consists of medicament in a gelatin or glycerogelatin base or in base of acacia, sucrose, and water (H. A. Lieberman, Pharmaceutical Dosage Forms: Tablets, Volume 1 (1980), Marcel Dekker, Inc., New York, N.Y.).

In a preferred embodiment, the present invention relates to oral care compositions in the form of a lozenge or pastille comprising an invertase, a beta-glucosidase, a glucoamylase, and at least one oral care ingredient, wherein the at least one oral care ingredient is selected from lubricant, bulking agent, sweetening agent, and flavoring agent. In a preferred embodiment, the oral care composition further comprises an alpha-amylase.

### Lubricants

The use of a lubricant in the manufacture of compressed lozenges is to facilitate the release of the lozenge from the die in which it is formed. The lubricant used in the present invention is a solid material which is not charged, and which will not interfere (e.g., complex) with the cationic antimicrobial. The material should preferably be water insoluble. One type of suitable material meeting these requirements is a non-toxic hydrocarbon fat or derivative. Examples include hydrogenated tallow and hydrogenated vegetable oil. Polyethylene glycols may also be used as a lubricant so long as they are solid materials which generally means having a molecular weight in the 4000 Da to 6000 Da range. These materials can also be used as a filler as noted below.

Mixtures of lubricants may also be used in the present invention. The lubricant is used at level of from about 0.1% to about 4.0% preferably from about 0.5% to about 2%.

### Lozenge Vehicle

The term “lozenge vehicle” is used herein to denote the material(s) which carries the active ingredients, i.e., the enzymes, as well as the lubricant. These materials are also known as bulking agents or fillers. Since the vehicle is non-cariogenic, the vehicle should be free of sucrose and similar materials.

Acceptable filler materials include mannitol, sorbitol, xylitol, polyethylene glycol and noncariogenic dextrans. The fillers may be used alone or in combination.

Mannitol is a naturally occurring sugar alcohol and is available as a fine powder. It has a sweetness of only about 50% of that of sucrose. However, mannitol's negative heat of solution enables it to impart a pleasant, cooling sensation in the mouth as the lozenge dissolves.

Sorbitol is a chemical isomer of mannitol and possesses a similar degree of sweetness. Its heat of solution, being negative, also provides for a pleasant, cooling sensation in the mouth. Sorbitol is available either as free flowing granules or as a crystalline powder. Polyethylene glycols (PEG's) can also be used in the present compositions. These materials are polymers of ethylene oxide with the generalized formula HOCH₂ (CH₂OCH₂)ₙCH₂OH. The use of PEG's alone is not favored but their use in combination with other fillers is acceptable. The molecular weights found most desirable are between 4000 Da and 6000 Da.

Fillers are generally used in the present invention at a level of from about 85% to about 99.8%, preferably from about 90% to about 98%, most preferably from about 94% to about 97%.

### Other Lozenge Components

Acceptable lozenges may be manufactured using just an active ingredient, the lubricant and the filler material as outlined above. However, to make the lozenges more acceptable from an aesthetic viewpoint, generally included are materials such as spray-dried or encapsulated flavors or liquid flavors adsorbed onto a suitable diluent. Spray-dried or encapsulated flavors are preferred. Suitable flavors include oil of peppermint, oil of wintergreen, oil of sassafras, oil of spearmint and oil of clove. Sweetening agents are also acceptable for use in the present compositions. Suitable agents include aspartame, acesulfame, saccharin, dextrose and levulose. Sweetening and flavoring agents are generally used in the compositions of this invention at levels of from about 0.1% to about 2%, preferably from about 0.25% to about 1.5%.

It is also acceptable to have a solid form of a water-soluble fluoride compound present in the present lozenges in an amount sufficient to give a fluoride concentration of from about 0.0025% to about 5.0% by weight, preferably from about 0.005% to about 2.0% by weight, to provide additionally anticaries effectiveness. Preferred fluorides are sodium fluoride, stannous fluoride, indium fluoride and sodium monofluorophosphate. The lozenges may also contain various active ingredients such as anti-microbial agents, Zn salts, fluorides, and urea (supra).

### Confectionaries and Candy

In a preferred embodiment, the present invention relates to oral care compositions in the form of a confectionary or candy comprising an invertase, a beta-glucosidase, a glucoamylase, and at least one oral care ingredient wherein the at least one oral care ingredient is selected from coloring agent, sweetening agent, flavoring agent, and oil-modifying agent. In a preferred embodiment, the oral care composition further comprises an alpha-amylase.

The preparation of confectionery formulations is historically well known and has changed little through the years. Confectionery items have been classified as either “hard” confectionery or “soft” confectionery. The volatile oil-modifying agent of the present invention can be incorporated by admixing the modifying agent into conventional hard and soft confections.

Hard confectionery may be processed and formulated by conventional means. In general, a hard confectionery has a base composed of a mixture of sugar and other carbohydrate bulking agents kept in an amorphous or glassy condition. This form is considered a solid syrup of sugars generally having from about 0.5% to about 1.5% moisture. Such materials normally contain up to about 92% corn syrup, up to about 55% sugar and from about 0.1% to about 5% water, by weight of the final composition. The syrup component is generally prepared from corn syrups high in fructose but may include other materials. Further ingredients such as flavorings, sweeteners, acidulants, colorants and so forth may also be added.

Such confectionery may be routinely prepared by conventional methods such as those involving fire cookers, vacuum cookers, and scraped-surface cookers also referred to as highspeed atmospheric cookers.

Fire cookers involve the traditional method of making a candy base. In this method, the desired quantity of carbohydrate bulking agent is dissolved in water by heating the agent in a kettle until the bulking agent dissolves. Additional bulking agent may then be added, and cooking continued until a final temperature of 145 to 156° C. is achieved. The batch is then cooled and worked as a plastic-like mass to incorporate additives such as flavor, colorants, and the like.

A high-speed atmospheric cooker uses a beat-exchanger surface, which involves spreading a film of candy on a heat exchange surface, the candy is heated to 165 to 170° C. in a few minutes. The candy is then rapidly cooled to 100 to 120° C. and worked as a plastic-like mass enabling incorporation of the additives, such as flavors, colorants, and the like.

In vacuum cookers, the carbohydrate bulking agent is boiled to 125 to 132° C., vacuum is applied, and additional water is boiled off without extra heating. When cooking is complete, the mass is a semi-solid and has a plastic-like consistency. At this point, flavors, colorants, and other additives are admixed in the mass by routine mechanical mixing operations.

The optimum mixing required to uniformly mix the flavors, colorants, and other additives during conventional manufacturing of hard confectionery is determined by the time needed to obtain a uniform distribution of the materials. Normally, mixing times of from 4 to 10 minutes have been found to be acceptable.

Once the candy mass has been properly tempered, it may be cut into workable portions or formed into desired shapes. A variety of forming techniques may be utilized depending upon the shape and size of the final product desired. A general discussion of the composition and preparation of hard confections may be found in H. A. Lieberman, Pharmaceutical Dosage Forms: Tablets, Volume 1 (1980), Marcel Dekker, Inc., New York, N.Y.

The apparatus useful in accordance with the present invention comprises cooking and mixing apparatus well known in the confectionery manufacturing arts, and election of the specific apparatus will be apparent to the artisan. In contrast, compressed tablet confections contain particular materials and are formed into structures under pressure.

These confections generally contain sugars in amounts up to about 95%, by weight of the composition, and typical tablet excipients such as binders and lubricants as well as flavoring agent, colorants and so forth. Like hard confectionery, soft confectionery may be utilized in this invention. The preparation of soft confections, such as nougat, involves conventional methods, such as the combination of two primary components, namely (1) a high boiling syrup such as corn syrup, hydrogenated starch hydrolysate or the like, and (2) a relatively light textured frappe, generally prepared from egg albumin, gelatin, vegetable proteins, such as soy derived compounds, sugarless milk derived compounds such as milk proteins, and mixtures thereof. The frappe is generally relatively light, and may, for example, range in density from about 0.5 to about 0.7 grams/cc.

The flavoring components of the confection are flavors having an associated bitter taste or other unpleasant after taste. These flavoring components may be chosen from natural and synthetic flavoring liquids such as volatile oils, synthetic flavor oils, flavoring aromatic and oils, liquids, oleoresins, or extracts derived from plants, leaves, flowers, fruits, stew, and combinations thereof. Non-limiting representative examples of volatile oils include spearmint oil, cinnamon oil, oil of wintergreen (methyl salicylate), peppermint oil, menthol, clove oil, bay oil, anise oil, eucalyptus oil, thyme oil, cedar leaf oil, oil of nutmeg, allspice oil, oil of sage, mace extract, oil of bitter almonds, and cassia oil. In addition, the confection may also contain artificial, natural, or synthetic flavors including fruit flavors such as vanilla, and citrus oils including lemon, orange, grape, lime and grapefruit and fruit essences including apple, pear, peach, grape, strawberry, raspberry, cherry, plum, pineapple, apricot and so forth individual and mixed.

Other useful flavorings include aldehydes and esters such as benzaldehyde (cherry, almond), citral, i.e., alpha-citral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyl-octanal (green fruit), and 2-dodecenal (citrus, mandarin), mixtures thereof and the like.

In the instance where sweeteners are utilized, the present invention contemplates the inclusion of those sweeteners well known in the art, including both natural and artificial sweeteners. The sweeteners may be chosen from the following non-limiting list: sugars such as sucrose, glucose (corn syrup), dextrose, invert sugar, fructose, and mixtures thereof, saccharin and its various salts such as the sodium or calcium salt; cyclamic acid and its various salts such as the sodium salt; the dipeptide sweeteners such as aspartame, dihydrachalcone compounds, glycyrrhizin; Stevia Rebaudiana (Stevioside); chloro-derivatives of sucrose; dihydroflavinol; hydroxyguaiacol esters; L-amino dicarboxylic acid gem-diamines; L-aminodicarboxylic acid aminoalkenoic acid ester amides; and sugar alcohols such as sorbitol, sorbitol syrup, mannitol, xylitol, and the like. Also contemplated is the synthetic sweetener 3,6-dihydro-6-methyl-1,2,3-oxathiazin-4-one-2,2-dioxide, particularly the potassium (acesulfame-K), sodium and calcium salts thereof.

The confection may also include a colorant. The colorants may be selected from any of the numerous dyes suitable for food, drug, and cosmetic applications, and known as FD&C dyes and the like. The materials acceptable for the foregoing spectrum of use are preferably water-soluble. Illustrative examples include indigoid dye, known as FD&C Blue No. 2, which is the disodium salt of 5,5′-indigotindisulfonic acid. Similarly, the dye known as FD&C Green No. 1 comprises a triphenylmethane dye and is the monosodium salts of 4-[4-N-ethyl-p-sulfobenzylami no)diphenylmethylane]-[1-(N-ethyl-N-p-sulfoniumbenzyl)-2-5-cyclohexadieneimine]. A full recitation of all FD&C and D&C dyes and their corresponding chemical structures may be found in the Kirk-Othmer Encyclopedia of Chemical Technology, in Volume 5.

The confectionary may also include a volatile oil-modifying agent such as capsicum oleoresin. An oil-modifying agent is present in an amount, which is undetected as a separate ingredient in the oral cavity, but nevertheless can modify sensory perception of the volatile oil. The oilmodifying agent is present in an amount of from about 1 to about 150 ppm of the confection. The capsicum is available from Capsicum minimum, Capsicum frutescens, Capsicum annuum, and similar varieties. Commercially, the fruits of capsicum are referred to as chilies or as peppers. These fruits are known for their extreme potency of bite, pungency, and characteristic odor.

With respect to confectionery compressed tablet formulations, such will contain a tablet granulation base and various additives such as sweeteners and flavors. The tablet granulation base employed will vary depending upon factors such as the type of base used, friability desired and other components used to make the final product. These confections generally contain sugars in amounts up to 95% by weight of the composition.

The confectionery compressed tablet may additionally include tablet excipients such as binders or lubricants, as well as flavoring agents, coloring agents, and volatile oils and volatile oilmodifying agents.

The variations that one may practice with regard to these confections are wide ranging and within the ability of those skilled in the art particularly with regard to the use of additional composition fillers, flavoring agents, the use of coloring agents, etc.

### External Oral Care Compositions

An external oral care formulation, e.g., denture cleaning solution, denture cleaning tablet, denture cleaning powder, and the like, may include ingredients and/or substances selected from the following categories:

| | | Amount |
|---|---|---|
| Type of component | Examples | (weight %) |
| Carrier liquid | Water, ethanol (18-27%), water/ | 0 to 95% |
| | ethanol mixture | |
| Disinfectant and | Sodium hypochlorite, Potassium | 0.001 to 95% |
| bleaching agents | Monopersulfate, Sodium Perborate | |
| Cleaning agents | Sodium bicarbonate, citric acid, | 0.001 to 95% |
| | sodium perborate, sodium | |
| | polyphosphate, potassium | |
| | monopersulfate | |
| Detergents and | Sodium lauryl sulfate (SLS) | 0.001 to 10% |
| surfactants | | |
| Foaming agents | Polyethylene glycols (PEGs) | 0.0001 to 10% |
| Preservatives | Sodium benzoate, citric acid, | 0.001 to 75% |
| Flavoring agents | Peppermint, spearmint | 0.001 to 5% |

In a preferred embodiment the at least on oral care ingredient is selected from the group consisting of carrier liquids, disinfectant and bleaching agents, cleaning agents, detergents and surfactants, foaming agents, preservatives, and flavoring agents.

In one aspect, the oral care compositions of the invention may also be included in filaments suitable for use in dental cleaning, e.g., filaments useful as dental floss. Preferably, the oral care composition is coated onto the exterior of the filament. Thus, in a preferred embodiment, the present invention relates to a filament comprising an oral care composition comprising an invertase, a beta-glucosidase, and a glucoamylase, wherein the filament is suitable for dental cleaning.

### Application of Oral Care Compositions

The oral care compositions of the invention are suitable for use in the treatment of oral disease, wherein prevention or removal of oral biofilm is desired. The compositions of the invention are particularly suitable for treating periodontal diseases and dental caries.

Periodontal disease, also known as gum disease, is a set of inflammatory conditions caused by bacterial infection and subsequent biofilm build-up on the test and the tissues surrounding the teeth. Periodontal disease may be divided in terms of severity into the following categories: gingivitis (including plaque-induced gingivitis), chronic periodontitis, aggressive periodontitis, periodontitis as a manifestation of systemic disease, necrotizing ulcerative gingivitis/periodontitis, abscesses of the periodontium, and combined periodontic-endodontic lesions. Periodontal disease may further be considered either localized or generalized depending on the extent of the affected area.

Dental caries, also known as tooth decay or cavities, is caused by organic acids, such as lactic acid, being released by certain biofilm-forming bacteria residing in the oral cavity, including Streptococcus mutans and some Lactobacillus species. Dental caries may be associated with further complications such as inflammation of the tissue around the teeth, tooth loss, and infection or abscess formation. Dental caries may be classified by location, etiology, rate of progression, and affected hard tissues, for instance according to the G.V. Black classification (class I, II, III, IV, V, and VI).

In one aspect, the present invention relates to an oral care composition comprising an invertase, a beta-glucosidase, a glucoamylase, and at least one oral care ingredient for use as a medicament. In a preferred embodiment, the oral care composition further comprises an alphaamylase.

In one aspect, the present invention relates to an oral care composition comprising an invertase, a beta-glucosidase, a glucoamylase, and at least one oral care ingredient for use in the treatment of oral disease. In a preferred embodiment, the oral care composition further comprises an alpha-amylase.

In a preferred embodiment, the present invention relates to an oral care composition comprising an invertase, a beta-glucosidase, a glucoamylase, and at least one oral care ingredient for use in the treatment of periodontal disease and/or dental caries. In a preferred embodiment, the oral care composition further comprises an alpha-amylase.

In one aspect, the present invention relates to use of an oral care composition comprising an invertase, a beta-glucosidase, a glucoamylase, and at least one oral care ingredient for treatment or prophylactic treatment of a human subject. In a preferred embodiment, the oral care composition further comprises an alpha-amylase.

In one aspect, the present invention relates to a method of treatment of a human subject, the method comprising administering an oral care composition comprising an invertase, a beta-glucosidase, a glucoamylase, and at least one oral care ingredient to the human subject. In a preferred embodiment, the oral care composition further comprises an alpha-amylase. In a preferred embodiment, the oral care composition is administered to the oral cavity of the human subject.

In one aspect, the present invention relates to a method for prevention or removing oral biofilm, the method comprising contacting the biofilm with an oral care composition comprising an invertase, a beta-glucosidase, a glucoamylase, and at least one oral care ingredient. In a preferred embodiment, the oral care composition further comprises an alpha-amylase. In one embodiment, the oral care composition is an external oral care composition, and the biofilm is located on an object; preferably the object is a denture. In one embodiment, object is located inside or outside the oral cavity.

### PREFERRED EMBODIMENTS

1) An oral care composition comprising an invertase, a beta-glucosidase, a glucoamylase, and at least one oral care ingredient.
2) The oral care composition according to embodiment 1, wherein the invertase, beta-glucosidase, and glucoamylase are of microbial original; preferably the invertase, beta-glucosidase, and glucoamylase are, independently, of bacterial or fungal origin.
3) The oral care composition according to any of embodiments 1-2, wherein the invertase, the beta-glucosidase, and the glucoamylase are each present in an effective amount; preferably an amount of from about 1 ppm to about 500 ppm.
4) The oral care composition according to any of embodiments 1-3, wherein the invertase is selected from the group consisting of:
   a) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:2 or to the polypeptide of SEQ ID NO:3;
   b) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:11 or SEQ ID NO:12;
   c) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:14 or SEQ ID NO:15;
   d) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:17 or SEQ ID NO:18;
   e) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:20 or SEQ ID NO:21;
   f) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:23 or SEQ ID NO:24;
   g) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:26 or SEQ ID NO:27;
   h) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:29 or SEQ ID NO:30;
   i) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:32 or SEQ ID NO:33;
   j) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:35 or SEQ ID NO:36;
   k) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:38 or SEQ ID NO:39;
   l) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:41 or SEQ ID NO:42;
   m) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:44 or SEQ ID NO:45; and
   n) a polypeptide having at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:47 or SEQ ID NO:48.
5) The oral care composition according to any of embodiments 1-4, wherein the invertase is selected from the group consisting of:
   a) a polypeptide comprising, consisting essentially of, or consisting of a mature polypeptide of SEQ ID NO:2 or SEQ ID NO:3;
   b) a polypeptide comprising, consisting essentially of, or consisting of a mature polypeptide of SEQ ID NO:11 or SEQ ID NO:12;
   c) a polypeptide comprising, consisting essentially of, or consisting of a mature polypeptide of SEQ ID NO:14 or SEQ ID NO:15;
   d) a polypeptide comprising, consisting essentially of, or consisting of a mature polypeptide of SEQ ID NO:17 or SEQ ID NO:18;
   e) a polypeptide comprising, consisting essentially of, or consisting of a mature polypeptide of SEQ ID NO:20 or SEQ ID NO:21;
   f) a polypeptide comprising, consisting essentially of, or consisting of a mature polypeptide of SEQ ID NO:23 or SEQ ID NO:24;
   g) a polypeptide comprising, consisting essentially of, or consisting of a mature polypeptide of SEQ ID NO:26 or SEQ ID NO:27;
   h) a polypeptide comprising, consisting essentially of, or consisting of a mature polypeptide of SEQ ID NO:29 or SEQ ID NO:30;
   i) a polypeptide comprising, consisting essentially of, or consisting of a mature polypeptide of SEQ ID NO:32 or SEQ ID NO:33;
   j) a polypeptide comprising, consisting essentially of, or consisting of a mature polypeptide of SEQ ID NO:35 or SEQ ID NO:36;
   k) a polypeptide comprising, consisting essentially of, or consisting of a mature polypeptide of SEQ ID NO:38 or SEQ ID NO:39;
   l) a polypeptide comprising, consisting essentially of, or consisting of a mature polypeptide of SEQ ID NO:41 or SEQ ID NO:42;
   m) a polypeptide comprising, consisting essentially of, or consisting of a mature polypeptide of SEQ ID NO:44 or SEQ ID NO:45; and
   n) a polypeptide comprising, consisting essentially of, or consisting of a mature polypeptide of SEQ ID NO:47 or SEQ ID NO:48.
6) The oral care composition according to any of embodiments 1-5, wherein the invertase comprises, consists essentially of, or consists of a mature polypeptide of SEQ ID NO:2 or SEQ ID NO:3.
7) The oral care composition according to any of embodiments 1-3, wherein the beta-glucosidase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:5 or to the polypeptide of SEQ ID NO:6; preferably the beta-glucosidase comprises, consists essentially of, or consists of a mature polypeptide of SEQ ID NO:5 or the polypeptide of SEQ ID NO:6.
8) The oral care composition according to any of claims 1-3, wherein the glucoamylase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to a mature polypeptide of SEQ ID NO:8 or to the polypeptide of SEQ ID NO:9; preferably the glucoamylase comprises, consists essentially of, or consists of a mature polypeptide of SEQ ID NO:8 or the polypeptide of SEQ ID NO:9.
9) The oral care composition according to any of embodiments 1-6 wherein the invertase has on par or improved thermal stability in the presence of at least one, e.g., at least two, at least three, at least four, at least five, at least six, at least seven, at least eight, at least nine, at least ten, or all, oral care ingredient(s) selected from the group consisting of benzoate (preferably sodium benzoate), EDTA, ethanol, fluoride (preferably sodium fluoride), glycerol, hydrogen peroxide, mannitol, phosphate (preferably sodium phosphate), SDS, sorbate (preferably potassium sorbate), and sorbitol.
10) The oral care composition according to embodiment 9, wherein the invertase has on par or improved thermal stability at pH 4-8; preferably at pH 5-7, more preferably at pH 5-6, most preferably at pH 5 and/or pH 6.
11) The oral care composition according to any of embodiments 9-10, wherein thermal stability is determined as described in Example 3.
12) The oral care composition according to any of embodiments 1-3 or 7, wherein the beta-glucosidase has on par or improved thermal stability in the presence of at least one, e.g., at least two, at least three, at least four, at least five, at least six, at least seven, at least eight, at least nine, at least ten, or all, oral care ingredient(s) selected from the group consisting of benzoate (preferably sodium benzoate), EDTA, ethanol, fluoride (preferably sodium fluoride), glycerol, hydrogen peroxide, mannitol, phosphate (preferably sodium phosphate), SDS, sorbate (preferably potassium sorbate), and sorbitol.
13) The oral care composition according to embodiment 12, wherein the beta-glucosidase has on par or improved thermal stability at pH 4-8; preferably at pH 5-7, more preferably at pH 5-6, most preferably at pH 5 and/or pH 6.
14) The oral care compositions according to any of embodiments 12-13, wherein thermal stability is determined as described in Example 3.
15) The oral care composition according to any of embodiments 1-3 or 8, wherein the glucoamylase has on par or improved thermal stability in the presence of at least one, e.g., at least two, at least three, at least four, at least five, at least six, at least seven, at least eight, at least nine, at least ten, or all, oral care ingredient(s) selected from the group consisting of benzoate (preferably sodium benzoate), EDTA, ethanol, fluoride (preferably sodium fluoride), glycerol, hydrogen peroxide, mannitol, phosphate (preferably sodium phosphate), SDS, sorbate (preferably potassium sorbate), and sorbitol.
16) The oral care composition according to embodiment 15, wherein the beta-glucosidase has on par or improved thermal stability at pH 4-8; preferably at pH 5-7, more preferably at pH 5-6, most preferably at pH 5 and/or pH 6.
17) The oral care composition according to any of embodiments 15-16, wherein thermal stability is determined as described in Example 3.
18) The oral care composition according to any of embodiments 1-6 or 9-11, wherein the invertase prevents formation of oral biofilm by at least 5%, e.g., 10%, 15%, 20%, 25%, 30%, 40%, 50%, 60%, 70%, 80%, 90%, or even 100%.
19) The oral care composition according to any of embodiments 1-3, 7, or 12-14, wherein the beta-glucosidase prevents formation of oral biofilm by at least 5%, e.g., 10%, 15%, 20%, 25%, 30%, 40%, 50%, 60%, 70%, 80%, 90%, or even 100%,
20) The oral care composition according to any of embodiments 1-3, 8, or 15-17, wherein the glucoamylase prevents formation of oral biofilm by at least 5%, e.g., 10%, 15%, 20%, 25%, 30%, 40%, 50%, 60%, 70%, 80%, 90%, or even 100%,
21) The oral care composition according to any of embodiments 18-20, wherein oral biofilm prevention is determined as described in Example 4 or Example 5.
22) The oral care composition according to any of embodiments 1-21, which further comprises an alpha-amylase.
23) The oral care composition according to embodiment 20, wherein the alpha-amylase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to the polypeptide of SEQ ID NO:50; most preferably wherein the alpha-amylase comprises, consists essentially of, or consists of the polypeptide of SEQ ID NO:50
24) The oral care composition according to any of embodiments 22-23, wherein the alphaamylase has on par or improved thermal stability in the presence of at least one, e.g., at least two, at least three, at least four, at least five, at least six, at least seven, at least eight, at least nine, at least ten, or all, oral care ingredient(s) selected from the group consisting of benzoate (preferably sodium benzoate), EDTA, ethanol, fluoride (preferably sodium fluoride), glycerol, hydrogen peroxide, mannitol, phosphate (preferably sodium phosphate), SDS, sorbate (preferably potassium sorbate), and sorbitol.
25) The oral care composition according to embodiment 24, wherein the alpha-amylase has on par or improved thermal stability at pH 4-8; preferably at pH 5-7, more preferably at pH 5-6, most preferably at pH 5 and/or pH 6.
26) The oral care composition according to any of embodiments 24-25, wherein thermal stability is determined as described in Example 3.
27) The oral care composition according to any of embodiments 22-246, wherein the alphaamylase removes oral biofilm by at least 5%, e.g., 10%, 15%, 20%, 25%, 30%, 40%, 50%, 60%, 70%, 80%, 90%, or even 100%.
28) The oral care composition according to embodiment 27, wherein oral biofilm removal is determined as described in Example 6.
29) The oral care composition according to any of embodiments 1-28 in the form of an internal oral care composition; preferably in the form of a toothpaste or toothpaste tablet, dental cream, mouthwash or mouthwash tablet, mouth rinse, lozenge, pastille, chewing gum, confectionary, or candy.
30) The oral care composition according to any of embodiments 1-28 in the form of an external oral care composition; preferably in the form of denture cleaning solution, denture cleaning tablet, or denture cleaning powder.
31) The oral care composition according to any of embodiments 1-30 for use as a medicament.
32) The oral care composition according to any of embodiments 1-30 for use in the treatment of oral disease; preferably for use in the treatment of periodontal disease and/or dental caries.
33) Use of an oral care composition according to any of embodiments 1-30 for treatment or prophylactic treatment of a human subject.
34) A method of treatment of a human subject, the method comprising administering an oral care composition according to any of embodiments 1-30 to the human subject; preferably the oral care composition is administered to the oral cavity of the human subject.
35) A method for preventing or removing oral biofilm, the method comprising contacting the oral biofilm with an oral care composition according to any of embodiments 1-30.
36) The method of embodiment 35, wherein the oral biofilm is located on an object, preferably a denture.
37) The method according to embodiment 34, wherein the denture is located inside or outside the oral cavity.
38) A method for reducing the risk of oral biofilm formation, the method comprising contacting the oral biofilm with an oral care composition according to any of embodiments 1-30.
39) A kit of parts comprising:
   a) an oral care composition according to any of embodiments 1-30; and
   b) instructions for use.

### EXAMPLES

### Materials and Methods

### Media and Solutions

YP+2% maltose medium was composed of 1% yeast extract, 2% bacto peptone and 2% maltose.

YP+2% glucose medium was composed of 1% yeast extract, 2% bacto peptone and 2% dextrose.

DAP4C-1 medium was composed 1.1% MgSO₄/7H₂O, 0.1% KH₂PO₄, 0.2% C₆H₈O₇/H₂O (citric acid), 2% dextrose, 1% maltose, 0.52% K₃PO₄/H₂O, 0.05% yeast extract, 0.05% trace metals, 0.1% DowFax 63N10, 0.05% CaCO₃

PDA plates were composed of 39 g Potato Dextrose Agar, 50 mL glycerol, 20 g agar and deionized water to 1 L. The medium was sterilized by autoclaving at 15 psi for 15 minutes (Bacteriological Analytical Manual, 8th Edition, Revision A, 1998).

LB agar plates were composed of 37 g LB agar (Sigma Aldrich L3027), 5 g soluble starch 0.5% (Merck 101252), 10 mL of K₂PO₄ 1 M, 20 mL of 20% glucose solution and deionized water to 1 L.

LB medium was composed of 25 g LB bouillon (Fluka L3152), and deionized water to 1 L. The medium was sterilized by autoclaving at 15 psi for 15 minutes (Bacteriological Analytical Manual, 8th Edition, Revision A, 1998).

COVE sucrose plates for Aspergillus transformants selection were composed of 342 g of sucrose, 20 g of agar powder, 20 mL of COVE salt solution, and deionized water to 1 L. The medium was sterilized by autoclaving at 15 psi for 15 minutes (Bacteriological Analytical Manual, 8th Edition, Revision A, 1998). The medium was cooled to 60° C., and 10 mM Na NO₃ were added

COVE salt solution was composed of 26 g of MgSO₄/7H₂O, 26 g of KCl, 76 g of KH₂PO₄, 50 mL of COVE trace metal solution, and deionized water to 1 L.

COVE trace metal solution was composed of 0.04 g of Na₂B₄O₇/10H₂O, 0.4 g of CuSO₄/5H₂O, 1.2 g of FeSO₄/7H₂O, 0.7 g of MnSO₄/H₂O, 0.8 g of Na₂MoO₄/2H₂O, 10 g of ZnSO₄/7H₂O, and deionized water to 1 L.

### Example 1a: Cloning and Expression of Invertases, Beta-Glucosidase, and Glucoamylase

SEQ ID NOs:1, 4, 7, 10, 13, 16, 19, 22, 25, 28, 31, 34, 37, 40, 43, and 46 were identified in their respective donor organisms and cloning was done according to the strategy described in US 2019/0225988 with three overlapping fragments for integration in the niiA/niiD locus of the ColS1300 strain using a DSMS system (described in US 2019/0225988).

ColS1300 protoplasts were prepared and transformed according to method described in WO 2012/003379 with approximatively 200 to 500 ng of each overlapping DNA fragments. Transformants were plated on COVE sucrose 10 mM NaNO₃ plates and selected for their ability to produce the respective mature polypeptides (SEQ ID NOs: 3, 6, 9, 12, 15, 18, 21, 24, 27, 30, 33, 36, 39, 42, 45, and 48, respectively) as confirmed by SDS-PAGE electrophoresis.

Fermentation was carried out in 250 mL baffled shake flasks filled with 100 mL of fermentation medium and inoculated with spores of the selected strains expressing the respective mature polypeptides. Fermentations were made at 30° C. under 150 rpm agitation for four days. Invertases (SEQ ID NOs: 3, 12, 15, 18, 21, 24, 27, 30, 33, 36, 39, 42, 45, and 48) were expressed using DAP4C-1 medium, beta-glucosidase (SEQ ID NO: 6) was expressed using YP2% glucose medium, and glucoamylase (SEQ ID NO: 9) was expressed using YP2% maltose medium. Culture broths were harvested by filtration using a 0.2 μm filter device.

Invertases and beta-glucosidase were purified by hydrophobic interaction chromatography. Integrity of the mature polypeptides was checked by SDS-PAGE electrophoresis and concentrations of the purified enzymes were determined by absorbance at 280 nm. Glucoamylase was not purified, but a ColS1300 host background fermented under similar conditions was used as negative control to exclude unwanted host background activities.

### Example 1b: Cloning and Expression of Alpha-Amylase

The DNA encoding the mature polypeptide of the alpha-amylase gene from Bacillus amyloliquefaciens (SEQ ID NO: 49) was ordered at Twist Bioscience. The synthetic DNA fragment was directionally assembled to a Bacillus expression vector described in WO12/025577 by the standard Golden Gate cloning method using Bsal and T4 DNA ligase enzymes. Briefly, the DNA encoding the mature peptide of the alpha-amylase gene was cloned in frame to a Bacillus licheniformis secretion signal with the sequence MKQQKRLYARLLTLLFALIFLLPHSAAAA (SEQ ID NO:51), replacing the native secretion signal of the alpha-amylase gene. Downstream of the gene, an affinity tag with sequence HHHHHH (SEQ ID NO:52) was added to enable the purification process.

The alpha-amylase fusion gene was integrated by homologous recombination into a Bacillus subtilis host cell genome upon transformation. Transformants were selected on LB media agar supplemented with 6 microgram of chloramphenicol per ml. One recombinant Bacillus subtilis clone containing the alpha-amylase expression construct was selected and was cultivated on a rotary shaking table in 500 ml baffled Erlenmeyer flasks containing 100 ml yeast extract-based media. After 3 days of cultivation time at 30° C., the enzyme containing supernatant was harvested by centrifugation and the enzymes was purified by His-tag purification.

The mature alpha-amylase (SEQ ID NO:50) with a C-terminal His-tag (SEQ ID NO:52) was purified by immobilized metal chromatography (IMAC) using Ni²′ as the metal ion on 5 mL HisTrap Excel columns (GE Healthcare Life Sciences). The purification took place at pH 7 and the bound protein was eluted with imidazole. The purity of the purified enzyme was checked by SDS-PAGE and the concentration of the enzyme was determined by measuring the absorbance at 280 nm after a buffer exchange in 50 mM HEPES, 100 mM NaCl, pH 7.0

### Example 2: Activity Assays

### Invertase Activity Assay

Invertase activity may be determined by incubation of 50 ppm invertase (0.05 mg/mL in 25 mM universal buffer comprising acetic acid, MES, HEPES, and glycine, pH 6) with sucrose substrate (1% w/v) for 60 min at 37° C. at 1400 rpm, and a sample without enzyme addition is used as control. After incubation, samples are centrifuged for 5 min at 16100×G at room temperature, and the supernatants are analyzed by liquid chromatography using a Dionex™-IC300 system with a PD10 column (ThermoFisher Scientific) with the following gradient and fructose and glucose (Sigma) as standard references:

| | | | % NaOH | % NaOAc |
|---|---|---|---|---|
| Time (min) | Flow (mL/min) | % Water | 0.5M | 0.5M |
| 0 | 0.8 | 97 | 3 | 0 |
| 4.5 | 0.8 | 96 | 4 | 0 |
| 7 | 0.8 | 93 | 7 | 0 |
| 10 | 0.8 | 80 | 15 | 5 |
| 25 | 0.8 | 50 | 15 | 35 |
| 28.1 | 0.8 | 85 | 15 | 0 |
| 29 | 0.8 | 90 | 10 | 0 |
| 30.1 | 0.8 | 97 | 3 | 0 |
| 33 | 0.8 | 97 | 3 | 0 |
| 33.1 | 0 | — | — | — |

For SEQ ID NO:3, the table below describes fructose release following incubation as measured by liquid chromatography as area under the curve in nano Coulombs per minute (nC*min), with the area under the curve being proportional to the fructose release. Liquid chromatography analysis revealed that the invertase can degrade sucrose into its constituent monosaccharides (fructose and sucrose), which is indicative of invertase activity.

| | Sucrose release | Fructose release | Glucose release |
|---|---|---|---|
| Invertase | (nC*min) | (nC*min) | (nC*min) |
| SEQ ID NO: 3 | none | 2329.69 | 917.42 |
| Control | 583.81 | none | none |
| (sucrose) | | | |

### Beta-Glucosidase Activity Assay

Beta-glucosidase activity may be determined via a colorimetric assay by incubating enzyme solution (20 μL of 0.1-10 μg/mL enzyme in 0.01% Triton X-100), assay buffer (50 μL, 100 mM acetic acid, 1 mM CaCl₂, 150 mM KCl, 0.01% Triton X-100, adjusted to pH 5 with NaOH), and substrate solution (50 μL, 2 mM 4-nitrophenyl-beta-D-glucopyranoside (Sigma N-7006) in 0.01% Triton X-100, prepared from a substrate stock solution of 100 mM 4-nitrophenyl-beta-Dglucopyranosidedissolved in MilliQ water) at 37° C. for 20 min, followed by addition of 50 μL stop solution (1 M Na₂CO₃), and measurement of the absorbance at 405 nm. A sample without enzyme solution is used as control.

### Glucoamylase Activity Assay

Glucoamylase activity may be determined via a colorimetric assay by incubating enzyme solution (20 μL of 3 μg/mL in 50 mM NaOAc, 0.02% Triton X-100, pH 4.3) and substrate solution (100 μL of 30 mg/mL maltodextrin (Sigma, De16.5-19.5) in 50 mM NaOAc, pH 4.3) in 96-well plates for 60 min at 37° C. at 800 rpm, wherein the 96-well plate has been preincubated with substrate solution for 3 min at 37° C. prior to addition of enzyme solution. The reaction is stopped by addition of stop solution (100 μL of 1 M Tris-HCl, pH 8.2. 10 μL of the reacted samples are transferred to another 96-well plate followed by addition of 200 μL Glucose CII test kit solution (Wako, 432-90913) to each well. The mixture is left at room temperature for 15 min, and the absorbance is measured at 505 nm. 10 μL glucose standards (0, 0.25, 0.50, 0.75, 1.0, 1.5, and 2.0 mg/mL glucose dissolved in H₂O) are used as controls.

### Alpha-Amylase Activity Assay I

Alpha-amylase activity may be determined using a reducing sugar assay employing, e.g., corn starch substrate. The number of reducing ends formed by the alpha-amylase hydrolysing the alpha-1,4-glycosidic linkages found in starch is determined by a reaction with p-hydroxybenzoic acid hydrazide (PHBAH). After reacting with PHBAH, the number of reducing ends can be measured by absorbance at 40 5 nm and the concentration of reducing ends is proportional to the alpha-amylase activity in the sample.

The corn starch substrate (3 mg/ml) is solubilised by cooking for 5 min in MilliQ water and cooled down before assaying. 50 μl activity buffer is mixed with 50 μL corn starch substrate and 50 μl enzyme and mixed followed by incubation for 5 min. The reaction is stopped by addition of 75 μl stop solution (Ka-Na-tartrate 50 g/L; NaOH 20 g/L; PHBAH 15 mg/mL) followed by incubation for 10 min at 95° C. and determination of absorbance at 405 nm. The alpha-amylase sample to be analyzed should be diluted so that the absorbance at 405 nm is between 0 and 2.2, which is within the linear range of the activity assay.

### Alpha-Amylase Activity Assay II

Alpha-amylase activity may be determined using method employing the Phadebas substrate (from for example Magle Life Sciences, Lund, Sweden). A Phadebas tablet includes interlinked starch polymers that are in the form of globular microspheres that are insoluble in water. A blue dye is covalently bound to these microspheres. The interlinked starch polymers in the microsphere are degraded at a speed that is proportional to the alpha-amylase activity. When the alphaamylase degrades the starch polymers, the released blue dye is water-soluble, and the concentration of the dye can be determined by measuring absorbance at 620 nm. The dye concentration is proportional to alpha-amylase activity.

The alpha-amylase sample to be analyzed is diluted in activity buffer with the desired pH value. One substrate tablet is suspended in 5 mL activity buffer under magnetic stirring. 30 μl diluted alpha-amylase sample is added to 150 μl substrate in a microtiter plate followed by mixing and incubation for 15 min at 37° C. The reaction is stopped by addition 30 μl of 1 M NaOH followed by mixing. The microtiter plate is centrifuged for 5 min at 4000×g, and 100 μl supernatant is transferred to a new microtiter plate. Absorbance is measured at 620 nm. The alpha-amylase sample to be analyzed should be diluted so that the absorbance at 620 nm is between 0 and 2.2, which is within the linear range of the activity assay.

### Example 3: Thermal Stability Measurements

### Preparation of Oral Care Formulations for Thermal Stability Measurements

The thermal stabilities or mid-point of the thermal unfolding transition (Tm) of selected invertases (SEQ ID NOs:3, 12, 15, and 36), beta-glucosidase (SEQ ID NO:6), glucoamylase (SEQ ID NO:9), and alpha-amylase (SEQ ID NO:50) were measured in the presence oral care components within the concentration range commonly used in oral care product formulations and selected oral care commercial products. The Tm parameter was used to evaluate the thermal stabilities as this is the temperature at which there are equal populations of folded and unfolded protein molecules and is a widely accepted parameter to use when evaluating thermal stability. Highly pure and biotechnology grade reagents were obtained from various suppliers and stock solutions were freshly prepared using MilliQ water. These formulation chemicals and their stock as well as the final concentrations used in the Tm measurement are listed in Table 1.

TABLE 1
Formulation chemical and stock solutions

| | | Product | Stock conc. |
|---|---|---|---|
| Chemicals | Source | Number | (mM) |
| Sodium benzoate | Sigma | 18106 | 100 |
| EDTA | Sigma | E5134 | 10 |
| Ethanol | VWR Chemicals | 20.824.296 | 10000 |
| Glycerol | Sigma | G5516 | 7600 |
| Sodium phosphate | Sigma | 71643 | 50 |
| Sorbitol | Sigma | S6021 | 3000 |
| SDS | Sigma | 5030 | 250 |
| Potassium sorbate | Sigma | 85520 | 100 |
| Sodium fluoride | Sigma | 919 | 100 |
| Hydrogen peroxide | Sigma | H1009 | 1000 |
| Mannitol | Sigma | M9546 | 1000 |

Purified preparations of enzyme samples were diluted to a stock concentration of 2 mg/ml prior to a further 10 times dilution in the oral care formulations consisting of individual formulation chemicals, citrate phosphate buffer (Mcllvaine buffer, see below) and MilliQ water corresponding to a final protein concentration of 0.2 mg/ml. Using robotics arm, all dilutions were made in a 384-well small volume deep well plate (Greiner Bio-One International, item number 784201) with a final volume of 70 μl and used for thermal stability measurements. The Tm measurements for each enzyme was performed close to physiological pH range of the oral cavity using Mcllvaine buffer at pH 5.0 and pH 6.0. 100 ml of Mcllvaine buffer pH 5.0 was prepared by mixing 51.50 ml 0.2 M Na₂HPO₄+48.50 ml 0.1 M citric acid, whereas 100 ml Mcllvaine buffer pH 6 prepared by mixing 63.15 ml 0.2 M Na₂HPO₄+36.85 ml 0.1 M citric acid.

### Determination of Tm

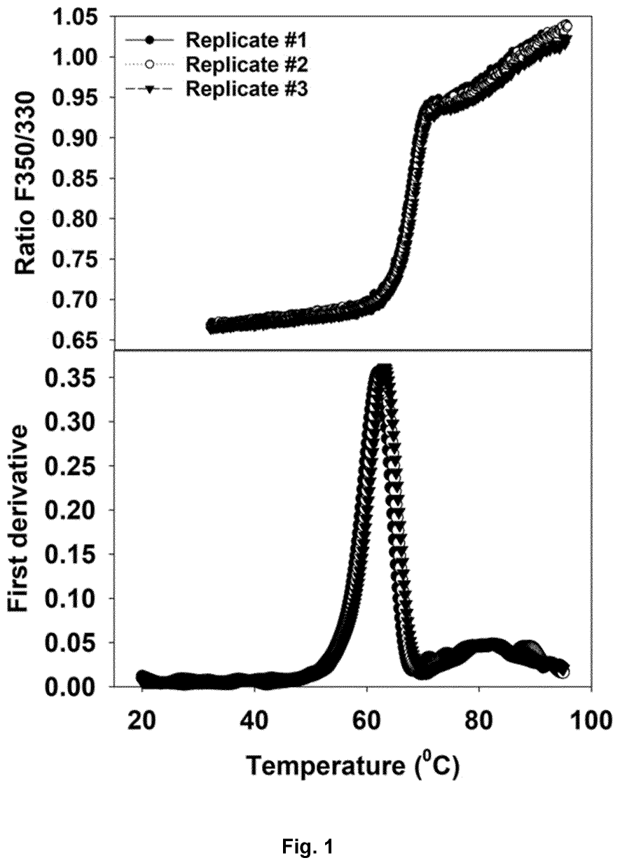

Thermal stability measurements were performed using a capillary based nano differential scanning fluorescence instrument (nanoDSF); Prometheus NT.Plex (NanoTemper Technologies GmbH, MQnchen, Germany). Standard nanoDSF grade capillary chips were used (Cat #: PRAC002 from NanoTemper Technologies). The enzyme samples were loaded into the capillaries (each sample in triplicate) by capillary action. The emission intensities at 330 and 350 nm were optimized by altering the LED power of the instrument to ensure sufficient signal. The fluorescence signals at 330 and 350 nm were monitored continuously as a function of temperature (heating rate used for thermal unfolding was 3.3° C. per minute from 20° C. to 95° C.). The data was analyzed using the PR.StabilityAnalysis 1.1.0.11077 software provided by the manufacturer. The analysis is model independent and simply takes the peak maximum of the first derivative which corresponds to the approximate thermal unfolding transition midpoint, defined as Tm (see FIG. 1).

### Reproducibility of Thermal Stability Data

FIG. 1 shows an example of the thermal stability data generated using the nanoDSF instrument. Panel A is an example of the data obtained (the ratio of the fluorescence emission at 350 nm to 330 nm) in triplicate for SEQ ID NO:3 as a function of temperature. Panel B shows the first derivative of the raw data in Panel A. The peak maximum in the first derivative plot corresponds to the mid-point of the thermal unfolding transition, referred to as Tm. In this example the Tm corresponds to 61.9° C. at pH 6.0 and is highly reproducible within the three replicates.

The data shown in FIG. 1 is an example of the type of data that was generated for the various enzymes in different formulations using nanoDSF. In all cases, the data showed a clear unfolding transition, and a clearly defined peak in the first derivative, and were highly reproducible.

### Thermal Stability of Invertases, Beta-Qlucosidase, Glucoamylase, and Alpha-Amylase in the Presence of Oral Care Formulation Components

Table 2 and Table 3 show the average thermal stabilities of invertase (SEQ ID NO:3), beta-glucosidase (SEQ ID NO:6), and glucoamylase (SEQ ID NO:9) derived from triplicate measurements at pH 5.0 and pH 6.0, respectively, in the presence of a range of commonly used oral care ingredients.

Table 4 and Table 5 show the average thermal stabilities of three invertases (SEQ ID NOs:36, 12, and 15) derived from triplicate measurements at pH 5.0 and pH 6.0, respectively, in the presence of a range of commonly used oral care ingredients.

Table 6 shows the average thermal stabilities of alpha-amylase (SEQ ID NO:50) derived from triplicate measurements at pH 5.0 and pH 6.0 in the presence of a range of commonly used oral care ingredients.

From these data, it is evident that these ingredients individually have no adverse effect on the thermal stability of invertase, beta-glucosidase, glucoamylase, and alpha-amylase, and that these enzymes have on par or even improved stability in the presence of these ingredients under conditions resembling those of the oral cavity, making the enzymes suitable for oral care formulation and application in the oral cavity.

TABLE 2
Thermal stability of SEQ ID NO: 3, 6, and 9 determined at pH 5

| | | Tm (° C.) at pH 5 | | |
|---|---|---|---|---|
| | Concentration | SEQ ID | SEQ ID | SEQ ID |
| Reagents | (mM) | NO: 3 | NO: 6 | NO: 9 |
| Control | 0 | 62.4 | 73.7 | 76.1 |
| EDTA | 1 | 62.9 | 73.7 | 76.2 |
| Ethanol | 1000 | 61.6 | 71.4 | 74.9 |
| Glycerol | 1000 | 62.8 | 71.8 | 77.2 |
| | 3250 | 64.4 | 70.8 | 78.2 |
| Hydrogen | 100 | 62.5 | 73.6 | 75.9 |
| peroxide | 500 | 60.7 | 73.1 | 73.4 |
| Potassium | 10 | 63.2 | 71.8 | 74.0 |
| sorbate | 35 | 64.1 | 69.5 | — |
| Mannitol | 250 | 62.9 | 73.8 | 76.7 |
| | 550 | 63.3 | 73.7 | 77.6 |
| Sodium | 10 | 63.2 | 73.2 | 75.5 |
| benzoate | 35 | 64.6 | 72.4 | 75.1 |
| Sodium | 20 | 62.6 | 73.7 | 76.3 |
| fluoride | 40 | 62.6 | 74.0 | 76.1 |
| Sodium | 5 | 62.5 | — | 73.1 |
| phosphate | 10 | 61.9 | — | 71.3 |
| SDS | 17 | 62.9 | 71.7 | 73.1 |
| | 170 | — | 71.6 | 72.8 |
| Sorbitol | 550 | 63.5 | 73.6 | 77.7 |
| | 2200 | 67.1 | 76.5 | 83.4 |

TABLE 3
Thermal stability of SEQ ID NO: 3, 6, and 9 determined at pH 6

| | | Tm (° C.) at pH 6 | | |
|---|---|---|---|---|
| | Concentration | SEQ ID | SEQ ID | SEQ ID |
| Reagents | (mM) | NO: 3 | NO: 6 | NO: 9 |
| Control | 0 | 61.9 | 67.5 | 72.4 |
| EDTA | 1 | 62.3 | 68.1 | 73.1 |
| Ethanol | 1000 | 61.1 | 66.0 | 71.3 |
| Glycerol | 1000 | 62.5 | 65.3 | 73.2 |
| | 3250 | 64.6 | 64.7 | 74.1 |
| Hydrogen | 100 | 61.9 | 67.5 | 72.2 |
| peroxide | 500 | 61.0 | 67.2 | 70.4 |
| Potassium | 10 | 62.1 | 67.6 | 71.0 |
| sorbate | 35 | 62.7 | 67.2 | — |
| Mannitol | 250 | 62.4 | 67.5 | 73.7 |
| | 550 | 62.9 | 67.8 | 74.5 |
| Sodium | 10 | 62.5 | 67.9 | 72.5 |
| benzoate | 35 | 63.5 | 68.8 | 72.3 |
| Sodium | 20 | 62.1 | 67.6 | 72.5 |
| fluoride | 40 | 62.0 | 67.6 | 72.9 |
| Sodium | 5 | 61.7 | 66.3 | 70.6 |
| phosphate | 10 | 61.0 | 66.2 | 69.6 |
| SDS | 17 | — | 67.3 | 70.6 |
| | 170 | — | 68.8 | 70.3 |
| Sorbitol | 550 | 62.8 | 67.5 | 75.1 |
| | 2200 | 67.4 | 71.9 | 81.2 |

TABLE 4
Thermal stability of SEQ ID NO:
36, 12, and 15 determined at pH 5

| | | Tm (° C.) at pH 5 | | |
|---|---|---|---|---|
| | Concentration | SEQ ID | SEQ ID | SEQ ID |
| Reagents | (mM) | NO: 36 | NO: 12 | NO: 15 |
| Control | 0 | 63.2 | 66.5 | 75.7 |
| EDTA | 1 | 63.0 | 66.5 | 75.9 |
| Ethanol | 1000 | 62.7 | 65.6 | 75.3 |
| Glycerol | 1000 | 63.1 | 66.7 | 76.9 |
| | 3250 | 63.7 | 66.2 | 78.6 |
| Hydrogen | 100 | 62.8 | 66.2 | 75.9 |
| peroxide | 500 | 61.6 | — | 76.2 |
| Potassium | 10 | 63.9 | 66.3 | 76.8 |
| sorbate | 35 | 64.0 | 66.1 | 77.2 |
| Mannitol | 250 | 63.0 | 67.2 | 76.4 |
| | 550 | 63.7 | 67.3 | 77.2 |
| Sodium | 10 | 62.9 | 66.4 | 76.4 |
| benzoate | 35 | 62.9 | 66.1 | 77.7 |
| Sodium | 20 | 63.0 | 66.4 | 75.9 |
| fluoride | 40 | 62.9 | 66.3 | 75.8 |
| Sodium | 5 | 63.0 | 65.7 | 77.0 |
| phosphate | 10 | 62.0 | 64.8 | 75.9 |
| SDS | 17 | — | — | 69.6 |
| | 170 | — | — | 69.0 |
| Sorbitol | 550 | 63.6 | 67.3 | 77.1 |
| | 2200 | 67.2 | 70.4 | 82.1 |

TABLE 5
Thermal stability of SEQ ID NO:
36, 12, and 15 determined at pH 6

| | | Tm (° C.) at pH 6 | | |
|---|---|---|---|---|
| | Concentration | SEQ ID | SEQ ID | SEQ ID |
| Reagents | (mM) | NO: 36 | NO: 12 | NO: 15 |
| Control | 0 | 62.5 | 65.2 | 76.5 |
| EDTA | 1 | — | — | — |
| Ethanol | 1000 | — | — | — |
| Glycerol | 1000 | 62.5 | 65.7 | 76.7 |
| | 3250 | 62.1 | 64.7 | 75.9 |
| Hydrogen | 100 | 63.3 | 66.4 | 77.7 |
| peroxide | 500 | 64.2 | 66.3 | 79.6 |
| Potassium | 10 | 62.8 | 65.3 | 76.4 |
| sorbate | 35 | 61.8 | 64.3 | 76.4 |
| Mannitol | 250 | 63.3 | 65.5 | 76.7 |
| | 550 | 64.5 | 65.3 | 76.1 |
| Sodium | 10 | 63.1 | 65.7 | 77.1 |
| benzoate | 35 | 63.8 | 66.4 | 78.0 |
| Sodium | 20 | 62.6 | 65.5 | 76.8 |
| fluoride | 40 | 62.6 | 65.5 | 77.2 |
| Sodium | 5 | 62.6 | 65.9 | 76.5 |
| phosphate | 10 | 62.7 | 65.5 | 76.3 |
| SDS | 17 | 61.6 | 64.3 | 75.4 |
| | 170 | 60.8 | 63.5 | 74.3 |
| Sorbitol | 550 | — | — | 75.8 |
| | 2200 | — | — | 75.4 |

TABLE 6
Thermal stability of SEQ ID NO: 50 determined at pH 5 and pH 6

| Reagents | Concentration (mM) | Tm (° C.) at pH 5 | Tm (° C.) at pH 6 |
|---|---|---|---|
| Control | 0 | 62.5 | 65.2 |
| EDTA | 1 | — | — |
| Ethanol | 1000 | — | — |
| Glycerol | 1000 | 62.5 | 65.7 |
| | 3250 | 62.1 | 64.7 |
| Hydrogen | 100 | 63.3 | 66.4 |
| peroxide | 500 | 64.2 | 66.3 |
| Potassium | 10 | 62.8 | 65.3 |
| sorbate | 35 | 61.8 | 64.3 |
| Mannitol | 250 | 63.3 | 65.5 |
| | 550 | 64.5 | 65.3 |
| Sodium | 10 | 63.1 | 65.7 |
| benzoate | 35 | 63.8 | 66.4 |
| Sodium | 20 | 62.6 | 65.5 |
| fluoride | 40 | 62.6 | 65.5 |
| Sodium | 5 | 62.6 | 65.9 |
| phosphate | 10 | 62.7 | 65.5 |
| SDS | 17 | 61.6 | 64.3 |
| | 170 | 60.8 | 63.5 |
| Sorbitol | 550 | — | — |
| | 2200 | — | — |

### Example 4: Human Saliva Biofilm Prevention Assay with Purified Enzymes

A human saliva biofilm prevention assay was carried out using the method described in WO 2020/099490 with some modifications. Briefly, biofilm was grown in 96-well plates in the presence of either 50 mM HEPES buffer with 100 mM NaCl, pH 7, as control, or an enzyme solution containing invertase (SEQ ID NO:3, 10 ppm and 60 ppm), beta-glucosidase (SEQ ID NO:6, 10 ppm and 60 ppm), glucoamylase (SEQ ID NO:9, 10 ppm and 60 ppm), alpha-amylase (SEQ ID NO:50, 60 ppm), or invertase, beta-glucosidase, glucoamylase, and alpha-amylase (SEQ ID NOs:3, 6, 9, and 50, 60 ppm of each enzyme), in 50 mM HEPES buffer with 100 mM NaCl, pH 7.

Plates were incubated at 37° C. for 24 hours without shaking in a Thermo Scientific™ Rectangular AnaeroBox™ Container under microaerophilic conditions (ThermoScientific AnaeroGen 2.5 L, #AN0025A). Enzyme and control samples were evaluated in eight replicates.

After incubation, planktonic bacteria were removed by two gentle washes with 100 μl 0.9% NaCl and biofilms were stained with 0.095% crystal violet solution for 15 min at room temperature. Plates were rinsed twice with 100 μL 0.9% NaCl and adhered dye was dissolved with a solution of 96% ethanol and 0.1% acetic acid. Absorbance was measured at 600 nm with a microplate reader (SpectraMax M3, Molecular Devices).

For data processing, the absorbance was taken to be proportional to the extent of remaining biofilm after enzyme or control treatment. The results were expressed as percentage of biofilm prevention and was calculated as follows:

100−((A600 nm enzyme treated sample)/(A600 nm buffer control treated sample)×100)

where A600 nm refers to the average of eight absorbance measurements at 600 nm of either enzyme or control treated samples. The results are listed in Table 7 and show that all three enzymes exhibit a biofilm prevention effect at 10 ppm and 60 ppm, with invertase having the highest activity at both concentrations.

TABLE 7
Prevention of biofilm formation (purified enzymes).

| | SEQ ID | % Prevention | Std | % Prevention | Std |
|---|---|---|---|---|---|
| Enzyme | NO: | (10 ppm) | Err | (60 ppm) | Err |
| Invertase | 3 | 77.5 | 0.9 | 76.5 | 1.1 |
| Beta-glucosidase | 6 | 35.4 | 3.7 | 69.2 | 0.8 |
| Glucoamylase | 9 | 24.0 | 4.1 | 58.9 | 1.2 |
| Alpha-amylase | 50 | — | — | 41.3 | 3.0 |
| Invertase + Beta- | 3 + 6 + | — | — | 85.9 | 0.6 |
| glucosidase + | 9 + 50 | | | | |
| Glucoamylase + | | | | | |
| Alpha-amylase | | | | | |

### Example 5: Human Saliva Biofilm Prevention Assay with Supernatants

A human saliva biofilm prevention assay was carried out using the method described in WO 2020/099490 with some modifications. Briefly, biofilm was grown in 96-well plates in the presence of either 50 mM HEPES buffer with 100 mM NaCl, pH 7 (as control) or an enzyme 5 solution containing invertases (SEQ ID NOs: 12, 15, 18, 21, 24, 27, 30, 33, 36, 39, 42, 45, and 48). The enzymes were evaluated using 10 μl supernatant samples from Aspergillus oryzeae recombinant strains expressing each enzyme.

Plates were incubated at 37° C. for 24 hours without shaking in a Thermo Scientific™ Rectangular AnaeroBox™ Container under microaerophilic conditions (ThermoScientific AnaeroGen 2.5 L, #AN0025A). Enzyme and control samples were evaluated in eight replicates.

After incubation, planktonic bacteria were removed by two gentle washes with 100 μl 0.9% NaCl and biofilms were stained with 0.095% crystal violet solution for 15 min at room temperature. Plates were rinsed twice with 100 μL 0.9% NaCl and adhered dye was dissolved with a solution of 96% ethanol and 0.1% acetic acid. Absorbance was measured at 600 nm with a microplate reader (SpectraMax M3, Molecular Devices).

For data processing, the absorbance was taken to be proportional to the extent of remaining biofilm after enzyme or control treatment. The results were expressed as percentage of biofilm prevention and was calculated as follows:

100−((A600 nm enzyme treated sample)/(A600 nm buffer control treated sample)×100)

where A600 nm refers to the average of eight absorbance measurements at 600 nm of either enzyme or control treated samples. The results are listed in Table 8 and show that all the evaluated invertases exhibit a potent biofilm prevention effect.

TABLE 8
Prevention of biofilm formation (using supernatant samples).

| | | % Prevention | |
|---|---|---|---|
| Enzyme | SEQ ID NO: | (10 μl) | Std Err |
| Control (host) | N/A | 0 | 1.2 |
| Invertase | 12 | 72 | 1.4 |
| Invertase | 15 | 71 | 1.8 |
| Invertase | 18 | 61 | 1.1 |
| Invertase | 21 | 67 | 1.7 |
| Invertase | 24 | 68 | 1.2 |
| Invertase | 27 | 70 | 0.7 |
| Invertase | 30 | 71 | 2.2 |
| Invertase | 33 | 60 | 3.0 |
| Invertase | 36 | 73 | 3.6 |
| Invertase | 39 | 74 | 1.0 |
| Invertase | 42 | 74 | 1.1 |
| Invertase | 45 | 61 | 5.2 |
| Invertase | 48 | 70 | 1.5 |

The biofilm removal assay was carried out using biofilm grown from human saliva in 96-well microtiter plates according to the method described in WO 2020/099490 with some modifications. Briefly, biofilm samples grown for 24 h were treated with either 200 μL citrate-phosphate buffer (Mcllvaine buffer, pH 6, prepared by mixing 12.63 ml 0.2 M Na₂HPOₐ+7.37 ml 0.1 M citric acid) as control, an enzyme solution containing invertase, beta-glucosidase, and glucoamylase (SEQ ID NOs:3, 6, and 9, 60 ppm of each enzyme) in Mcllvaine buffer, pH 6, or an enzyme solution containing invertase, beta-glucosidase, glucoamylase, and alpha-amylase (SEQ ID NOs:3, 6, 9, and 50, 60 ppm of each enzyme) in Mcllvaine buffer, pH 6, for 30 min at 37° C., 50 rpm shaking. Enzyme and control samples were carried out in eight replicates.

After enzyme treatment, samples were rinsed twice with 200 μL 0.9% sodium chloride and stained with 0.095% crystal violet solution for 15 min at room temperature. Samples were rinsed three times with 200 μL 0.9% sodium chloride and the dye was extracted with 33% acetic acid solution and absorbance at 600 nm was measured.

For data processing, the absorbance was taken to be proportional to the extent of remaining biofilm after enzyme or control treatment. The results were expressed as percentage of biofilm removal and was calculated as follows:

100−((A600 nm enzyme treated sample)/(A600 nm buffer control treated sample)×100),

where A600 nm refers to the average of eight absorbances measured at 600 nm using SpectraMax M3, Molecular Devices, of either enzyme or control treated samples. The results are listed in Table 9 and show that the combination of invertase, beta-glucosidase, glucoamylase, and alpha-amylase provides a significant improvement in biofilm removal compared to the combination of invertase, beta-glucosidase, and glucoamylase.

TABLE 9
Removal of human biofilm.

| | | % Removal | |
|---|---|---|---|
| Enzyme | SEQ ID NO: | (60 ppm) | Std Err |
| Invertase + Beta- | 3, 6, 9 | 1.6 | 3.5 |
| glucosidase + | | | |
| Glucoamylase | | | |
| Invertase + Beta- | 3, 6, 9, 50 | 22.0 | 1.0 |
| glucosidase + | | | |
| Glucoamylase + Alpha- | | | |
| amylase | | | |

1-15. (canceled)

16: An oral care composition comprising an invertase, a beta-glucosidase, a glucoamylase, and at least one oral care ingredient.

17: The oral care composition according to claim 16, wherein the invertase, beta-glucosidase, and glucoamylase are of microbial original.

18: The oral care composition according to claim 17, wherein the invertase, beta-glucosidase, and glucoamylase are, independently, of bacterial or fungal origin.

19: The oral care composition according to claim 16, wherein the invertase, the beta-glucosidase, and the glucoamylase are each present in an effective amount.

20: The oral care composition according to claim 16, wherein the invertase, the beta-glucosidase, and the glucoamylase are each present in an amount of from about 1 ppm to about 500 ppm.

21: The oral care composition according to claim 16, wherein the invertase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to the mature polypeptide of SEQ ID NO: 2 or to the polypeptide of SEQ ID NO: 3.

22: The oral care composition according to claim 16, wherein the invertase comprises, consists essentially of, or consists of a mature polypeptide of SEQ ID NO: 2 or the polypeptide of SEQ ID NO: 3.

23: The oral care composition according to claim 16, wherein the beta-glucosidase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to the mature polypeptide of SEQ ID NO: 5 or to the polypeptide of SEQ ID NO: 6.

24: The oral care composition according to claim 16, wherein the beta-glucosidase comprises, consists essentially of, or consists of a mature polypeptide of SEQ ID NO: 5 or the polypeptide of SEQ ID NO: 6.

25: The oral care composition according to claim 16, wherein the glucoamylase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to the mature polypeptide of SEQ ID NO: 8 or to the polypeptide of SEQ ID NO: 9.

26: The oral care composition according to claim 16, wherein the glucoamylase comprises, consists essentially of, or consists of a mature polypeptide of SEQ ID NO: 8 or the polypeptide of SEQ ID NO: 9.

27: The oral care composition according to claim 16, wherein the invertase has on par or improved thermal stability in the presence of at least one oral care ingredient selected from the group consisting of benzoate, EDTA, ethanol, fluoride, glycerol, hydrogen peroxide, mannitol, phosphate, SDS, sorbate, and sorbitol.

28: The oral care composition according to claim 16, wherein the beta-glucosidase has on par or improved thermal stability in the presence of at least one oral care ingredient selected from the group consisting of benzoate, EDTA, ethanol, fluoride, glycerol, hydrogen peroxide, mannitol, phosphate, SDS, sorbate, and sorbitol.

29: The oral care composition according to claim 16, wherein the glucoamylase has on par or improved thermal stability in the presence of at least one oral care ingredient selected from the group consisting of benzoate, EDTA, ethanol, fluoride, glycerol, hydrogen peroxide, mannitol, phosphate, SDS, sorbate, and sorbitol.

30: The oral care composition according to claim 16, which further comprises an alpha-amylase.

31: The oral care composition according to claim 30, wherein the alpha-amylase has at least 70%, e.g., at least 75%, at least 80%, at least 85%, at least 90%, at least 91%, at least 92%, at least 93%, at least 94%, at least 95%, at least 96%, at least 97%, at least 98%, at least 99%, or 100%, sequence identity to the polypeptide of SEQ ID NO: 50.

32: The oral care composition according to claim 30, wherein the alpha-amylase comprises, consists essentially of, or consists of the polypeptide of SEQ ID NO: 50

33: A method for preventing or removing oral biofilm, the method comprising contacting the oral biofilm with an oral care composition according to claim 16.

34: A method for reducing the risk of oral biofilm formation, the method comprising contacting the oral biofilm with an oral care composition according to claim 16.

35: A kit of parts comprising:
a) an oral care composition according to claim 16; and
b) instructions for use.
